(19) United States
(12) Patent Application Publication
(10) Pub. No.: US 2014/0357706 A1
(43) Pub. Date: Dec. 4, 2014

(54) Stearate Compounds

(71) Applicant: THE UAB RESEARCH FOUNDATION, Birmingham, AL (US)

(72) Inventor: Robert W. Hardy, Hoover, AL (US)

(73) Assignee: THE UAB RESEARCH FOUNDATION, Birmingham, AL (US)

(21) Appl. No.: 14/350,549

(22) PCT Filed: Oct. 15, 2012
(86) PCT No.: PCT/US12/60282

§ 371 Date: Apr. 8, 2014

Related U.S. Application Data
(60) Provisional application No. 61/546,616, filed on Oct. 13, 2011.

(51) Int. Cl. A61K 31/20; A23L 1/30; A61K 31/337

(52) U.S. Cl. CPC A61K 31/20; A61K 31/337; A23L 1/3008
USPC 514/449; 514/558; 554/1

(57) ABSTRACT

Preparation and methods of treating and preventing visceral adiposity and cancer are provided involving the administration of stearate to a subject. It has been unexpectedly discovered that the fatty acid stearate, when introduced in the diet, reduces the amount of visceral fat in the body without decreasing overall body weight or causing measurable negative side effects. It has also been unexpectedly discovered that dietary stearate prevents cancer in healthy subjects and reduces both tumor size and metastasis in subjects already afflicted with cancer.

### PRIORITY CLAIM

This application claims priority to U.S. provisional patent application No. 61/546,616 filed Oct. 13, 2011.

### BACKGROUND ART

Chronic disease related to diet imposes a significant mortality and morbidity burden on the human population. Obesity and related conditions continue to increase in prevalence in many developed countries, despite advances in understanding the relationship between obesity and numerous health problems. A subject's obesity is commonly measured as the ratio of total body fat mass to total body mass (total body fat content). The ratio of abdominal (visceral) fat mass to total body mass has been shown to be more predictive of health problems than is a subject's total body fat content. Consequently there is a pressing need for approaches to helping people lower not only their total body fat content, but especially their visceral fat content.

Obesity is also associated with problems with the regulation of glucose metabolism, such as diabetes and “metabolic syndrome.” Diabetes is a widespread and growing problem. In 2011, the U.S. National Institute of Diabetes and Digestive and Kidney Diseases estimated that 25.8 million people of all ages in the United States suffered from diabetes (over 8% of the U.S. population). More troubling is the observation that 79 million persons aged 20 and up in the United States are pre-diabetic, and likely to develop diabetes. From 1997 to 2007 the rate of type 2 diabetes doubled in the United States.

Dietary saturated fats are a known risk factor for many chronic diseases, including cardiovascular disease and cancer. Saturated fat consumption increases total serum cholesterol, and low-density lipoproteins (LDL), which are indicators of impending atherosclerotic disease. Saturated fat consumption is also associated with elevated risks of various types of cancers, including prostate cancer, breast cancer, and cancer of the small intestine. The current scientific understanding is that the consumption of saturated fat must be reduced to improve public health. The public health agencies of numerous countries recommend sharply limiting the dietary intake of saturated fat, including Health Canada, the U.S. Department of 5 Health and Human Services, the U.K. Food Standards Agency, the Australian Department of Health and Aging, the Singapore Government Health Promotion Board, the Indian Government Citizens Health Portal, the New Zealand Ministry of Health, the Food and Drugs Board of Ghana, the Guyana Ministry of Health, and the Hong Kong Center for Food Safety.

The U.S. Centers for Disease Control and Prevention concluded in a 2004 report that “Continuing efforts to decrease saturated fat intake are important to reduce the risk for cardiovascular disease and should include assessment of fat intake in grams in addition to fat intake as a percentage of kcals.” MMWR 53(04):80-82.

### DISCLOSURE OF THE INVENTION

The following presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

Stearate is an 18 carbon saturated fatty acid (C18:0) that occurs in many animal and vegetable fats and oils. It is an important constituent of milk fats, lard, and cocoa and shea butters. Stearate was first described by Michel Eugene Chevreul in 1823, and its name comes from Greek for “hard fat,” reflecting the fact that stearate forms a waxy solid. Some studies have suggested that diseases associated with the consumption of saturated fat are less likely to be caused by fats containing stearate groups. However, to date stearate has never been effectively used to treat or prevent disease.

It has been unexpectedly discovered that dietary stearate is a potent agent for the treatment and prevention of diseases, specifically those related to fat and sugar metabolism, and cancer.

It has been discovered that stearate has beneficial effects on fat and sugar metabolism. Specifically, it has been discovered that stearate: selectively reduces visceral fat content in animals, without affecting the animal's overall fat content or body weight; selectively induces apoptosis of visceral preadipocytes without affecting mature adipocytes in vitro; and reduces blood glucose and leptin concentrations.

It has also been unexpectedly discovered that stearate inhibits the cell cycle progression of tumor cells both in vivo and in vitro, and that dietary stearate reduces the incidence, number, and size of mammary tumors in vivo. Furthermore, when used in conjunction with chemotherapeutic agents, stearate reduces the incidence and/or severity of cancer in vivo if administered either before or after tumorigenesis.

The disclosure provides a dietary supplement comprising a substantial amount of a stearate compound, wherein said stearate compound is neither a naturally occurring triglyceride compound nor a naturally occurring phospholipid compound.

The disclosure also provides a food item containing a substantial amount of a stearate compound, wherein said stearate compound is neither a naturally occurring triglyceride compound nor a naturally occurring phospholipid compound.

A pharmaceutical preparation is provided, comprising a therapeutically effective amount of a stearate compound.

A method of improving or maintaining the health of a subject provided, the method comprising administering to the subject a stearate compound, other than a naturally occurring triglyceride compound or a naturally occurring phospholipid compound, in an amount equal to a significant fraction of the subject's total dietary lipid intake.

A method of inhibiting the cell cycle progression of a cell is provided, said method comprising contacting the cell with an inhibitory effective amount of a stearate compound, other than a naturally occurring triglyceride compound or a naturally occurring phospholipid compound.

A method of inducing apoptosis in a visceral pre-adipocyte cell is provided, comprising contacting the cell with an effective amount of a stearate compound, other than a naturally occurring triglyceride compound or a naturally occurring phospholipid compound.

### BRIEF DESCRIPTION OF THE DRAWINGS

FIG. 1. Average daily caloric intake and average weekly body weight. (A) The stearate diet group consumed slightly more calories than other dietary groups daily (*, stearate vs. all other diets, p<0.01). The low fat diet group consumed slightly less calories than the other dietary groups daily (#, low fat vs. all other diet groups, p<0.007). (B) Nevertheless, there were no significant changes in body weight among the four experimental groups throughout the study. The initial body weight was 15.3±2.6 g (p=0.277) and the final body weight was 25.0±2.3 g (p=0.203).

FIG. 2. Body composition measured by Dual-energy X-ray absorptiometry (DXA) at week 18. Total body fat (TBF), total body lean mass (TBLM) and bone mineral density (BMD) were assessed. (A) Mice on the stearate diet had slightly but significantly decreased TBF compared to the low fat group (*, p=0.003). (B) Mice on the stearate diet also had a corresponding increase in TBLM when compared to the other diet groups (*, p<0.01). (C) Mice on the stearate diet had a significantly reduced BMD compared to all other experimental groups (*, p<0.001) while the safflower oil diet minimally but significantly raised BMD compared to the low fat group (p=0.023).

FIG. 3. Abdominal fat and organ weight. (A) Abdominal fat images are representatively demonstrated from each experimental group. Eighteen weeks post-diet mice from the stearate group had significantly reduced abdominal fat as compared to mice in the low fat and corn oil groups. (B) Mice on the stearate diet had significantly less abdominal fat compared to the low fat and corn oil groups (*, p<0.01). (C) Mice on the stearate diet had slightly reduced kidney weight when compared to other experimental groups (*, stearate vs. low fat, p=0.024; corn oil, p=0.003; safflower, p=0.012).

FIG. 4. The size of adipocytes from abdominal fat. Histopathology of representative sections of abdominal fat from mice fed: a low fat diet (A), corn oil diet (B), safflower oil diet (C) or stearate diet (D) all at the same low power magnification (25×). The size of the adipocytes is smaller in the section from the stearate diet group. (E) The average area of each adipocyte was measured by histomorphometric techniques. Mice on the low fat diet had significantly larger adipocytes as compared to the stearate, corn oil and safflower groups (*, p<0.01).

FIG. 5. Histopathology of kidneys. Representative H&E stained sections of kidneys from mice fed either a low fat diet (A), corn oil diet (B), safflower oil diet (C) or stearate diet (D). All kidneys examined regardless of diet were without significant histopathologic abnormalities.

FIG. 6. Histopathology of liver. Representative H&E stained sections of liver from mice fed either a low fat diet (A), corn oil diet (B), safflower oil diet (C) or stearate diet (D). Again all sections were essentially normal.

FIG. 7. Serum biomarker analysis. Serum concentrations of glucose, leptin and MCP-1 were measured. (A) Mice on the stearate diet had significantly reduced serum glucose compared to all other experimental groups (*, stearate vs. low fat, p=0.006; corn oil, p=0.039; safflower oil, p<0.001). Mice on the corn oil diet also had a significantly reduced level of glucose as compared to the safflower group (p=0.034). (B) Mice on the high fat diets had significantly reduced level of leptin compared to the low fat group (*, low fat vs. stearate, p<0.001; corn oil, p=0.014; safflower oil, p<0.001). Mice on the stearate and safflower oil diets also had significantly lower level of leptin when compared to the corn oil group (p=0.015 and 0.003, respectively). (C) Mice on the stearate diet had significantly increased level of MCP-1 compared to the low fat and safflower oil groups (*, p=0.003 and 0.019, respectively). Mice on the low fat diet also had significantly reduced level of MCP-1 compared to the corn oil group (p=0.032).

FIG. 8. Effect of dietary stearate on 3T3L1 cell differentiation. Representative oil red O and hematoxylin stained control (A) and stearate treated 3T3L1 cells (B). The ratio of differentiated to unconverted adipocytes was similar in the two experimental groups. (C) The percentage of differentiated adipocytes was calculated and no significant difference was found. (D) The oil red O was eluted from the cells and the OD value was measured. Again no difference was observed between stearate and any of the other groups.

FIG. 9. Effects of 50 μM stearate, oleate or linoleate on necrosis and apoptosis of differentiated 3T3L1 adipocytes. Trypan blue stain was used to detect cell death and cytotoxicity was assessed by measurement of lactate dehydrogenase (LD) concentration in the medium. Flow cytometry was used to quantify the necrosis and apoptosis. (A) The trypan blue stain showed that there were no significant changes in the percentage of dead cells when the adipocytes were treated with stearate, oleate or linoleate throughout the study. (B) Cytotoxicity detection similarly showed no significant changes among the three experimental treatment groups. (C) There were no significant changes in the percentage of dead cells detected by flow cytometry when adipocytes were treated with stearate, oleate or linoleate. (D) Similarly, flow cytometry revealed no significant changes in the percentage of apoptotic cells among the three experimental treatment groups.

FIG. 10. Effects of 50 μM stearate, oleate and linoleate on cell death and apoptosis of 3T3L1 preadipocytes. Cell death, necrosis, apoptosis and cytotoxicity were performed as described in FIG. 9. (A) Trypan blue staining showed that the percentages of dead cells were significantly increased after a 48 hour treatment with stearate (*, p<0.01, compared to control). In contrast, oleate or linoleate had no significant changes over time. (B) Cytotoxicity was significantly increased after 24 hours of treatment with stearate (*, p<0.01, compared to control). It was also significantly decreased after 24 hours of oleate treatment (#, p<0.01, compared to control) but not after 48 hrs. Cytotoxicity did not change significantly with linoleate treatment. (C) Flow cytometry revealed an increase in dead cells after 48 hours treatment with stearate (*, p<0.01, compared to control), while oleate significantly decreased dead cells at 48 hours (#, p<0.01, compared to control). In contrast, linoleate had no significant effects over time. (D) Apoptotic cells were significantly increased with stearate treatment (*, p<0.01, compared to control) and decreased with oleate (#, p<0.01, compared to control). No significant changes were observed with linoleate treatment. (E) In parallel stearate increased caspase-3 activity at 48 hours (*, p<0.05, compared to control).

FIG. 11. Stearate alters the cell cycle in Hs578T cells largely in G1 and to a lesser 5 degree in G2. Cell cycle analysis of Hs578T cells by flow cytometry. See Materials and methods for details. Bar chart shows the average percentage of cells in nine independent experiments. Error bars indicate standard error of the mean; P<0.001, significant differences compared with control (CTL); P<0.001, significant differences compared with 1 nM epidermal growth factor control (EGF/CTL). ST 5 50 IM stearate, STM 5 50 IM stearate in complete medium and ST/EGF 5 50 IM stearate plus 1 nM EGF.

FIG. 12. (A) Stearate increased the cell cycle inhibitors p21^{CIP1/WAF1} and p27^{KIP} and decreased phosphorylated Cdk2. Densitometry of all time points − and [symbol] stearate, n 5 3, t-test, p21, P=0.01, pCdk2, P=0.04, p27, P=0.013. Using a repeated-measures model, there is a significant group*time effect P=0.0002 (n=3, data not shown). A representative immunoblot is shown. (B) Stearate (ST) increased the expression of p21^{CIP1/WAF1} but not p27^{KIP}. Bar charts represent the mean mRNA levels normalized to the 18S rRNA expression. Error bars indicate standard error of the mean; P<0.05, significant increase compared with control (n=3).

FIG. 13. Activation of Ras and ERK by stearate. (A) Stearate increased the binding of GTP to Ras with or without EGF. # indicates that Hs578T cells were incubated in starvation medium only. Ras-GTP was found to be significantly increased by stearate in each of two experiments when the four time points + and − stearate treatment were compared by densitometry (Experiment 1, P=0.045; Experiment 2, P=0.012; for both experiments P=0.018). (B) Immunoblot shows increased pERK after treatment with stearate. (C) ERK inhibitor PD98059 reversed stearate-induced upregulation of p21^{CIP1/WAF1}, but not p27^{KIP1}, and partially blocked stearate-induced dephosphorylation of pCdk2

FIG. 14. Decreased Rho activation and expression induced by stearate. (A) Epidermal growth factor (EGF)-induced Rho activation was inhibited by stearate pretreatment. # represents that cells were incubated in starvation medium without EGF for 24 h used as control. Rho-GTP was found to be significantly reduced by stearate treatment in each of three experiments when the four time points with and without stearate treatment were compared by densitometry (Experiment 1, P=0.045; Experiment 2, P=0.011; Experiment 3, P=0.032; for all three experiments P=0.0018). A representative experiment is shown. (B) RT-PCR for RhoA, RhoB, and RhoC when Hs578T cells were treated as indicated for 48 h. Bar charts represent the mean mRNA levels normalized to the GAPDH expression and relative to control samples without stearate. Error bars indicate standard error of the mean, P<0.05, significant increase compared with control (n=4). Constitutively active RhoB and RhoC inhibited stearate-induced p21^{CIP1/WAF1} (C) and p27^{KIP} (D) in Hs578T cells.

FIG. 15. Dietary stearate inhibits carcinogenesis in the NMU carcinogen-induced rat breast cancer model. All error bars indicate standard error of the mean. (A) Animals were monitored weekly for the development of mammary tumors after NMU injection. Fewer animals in the stearate and safflower diets developed palpable tumors. P<0.05, significantly decreased compared with the low-fat diet group. (B) Dietary stearate reduced tumor burden as measured by the average number of tumors developed per rat (n=30-35), P>0.02, and (C) as measured by tumor weight per rat (n=30-35), P<0.01, compared with the low-fat diet group. The safflower diet group did not reach statistical difference compared with low-fat diet (P=0.057) for tumor weight. (D) Tumors were classified into four categories: intraductal proliferations (IDP), tubular adenoma (TA), ductal carcinoma in situ (DCIS) and adenocarcinoma (CA). Compared with low-fat treatment.

FIG. 16. Dietary stearate inhibits Rho mRNA expression in the NMU carcinogen-induced rat breast cancer model. RT-PCR for Rho in microdissected tumor cells from tumor frozen sections showed that RhoA, RhoB and total Rho mRNA expression significantly decreased in the dietary stearate and safflower groups. Bar charts represent the mean mRNA levels normalized to the GAPDH expression and differences are relative to low-fat group (n=5). Error bars indicate standard error of the mean, P<0.01.

FIG. 17. Experimental Timetable. (A) Experiment 1: Nude mice were placed on either a control (low fat) diet, a corn oil diet, a safflower oil diet, or a stearate diet 3 weeks prior to injection of cancer cells. The tumors were allowed to reach an approximate mean tumor diameter of 10-12 mm (253.6-904.8 mm³) at which time the primary tumors were removed (about 9 weeks post-injection). Chemotherapy with paclitaxel started 1 week after the surgery. After that, the animals were allowed to develop metastases for about 3 weeks, sacrificed and the lungs were collected. (B) Experiment 2: Diet therapy was initiated at the same time as chemotherapy, which is one month after the primary tumor was removed. Before diet therapy, the mice were fed with control diet. According to the size of primary tumors before surgery, the mice were divided into six groups evenly: a control diet group, a corn oil diet group, a stearate diet group, a control diet plus PTX group, a corn oil diet plus PTX group, and a stearate diet plus PTX group.

FIG. 18. Effect of diet plus chemotherapy on the incidence of lung metastasis. The 15 number of mice with lung metastases was counted following necropsy, and the percentage of mice with lung metastasis in different groups was compared. (A) In experiment 1, mice on stearate diet had significantly decreased incidence of lung metastasis compared to the control and corn oil diet groups. (n=25-30 animals per diet; *, p<0.05, stearate VS. control diet group; #, p<0.05, stearate VS. corn oil diet group). (B) In experiment 2, mice on both stearate and corn oil diet had significantly reduced incidence of lung metastases compared to control diet group. (n=25-30 animals per diet; *, p<0.01, stearate or corn oil diet groups VS. control diet group; #, p<0.01, stearate diet plus PTX or corn oil diet plus PTX groups VS. control diet plus PTX group). Mice on PTX had significantly lower incidence of lung metastases in different diet conditions (&, p<0.01, control diet plus PTX group VS. control diet group, corn oil diet plus PTX group VS. corn oil diet group, stearate diet plus PTX group VS. stearate diet group).

FIG. 19. Diet therapy and chemotherapy on the number of lung metastasis. The number of lung metastatic tumors per animal was counted and compared following necropsy. (A) In experiment 1, mice from the stearate diet group had significantly decreased number of lung metastases compared to those from control diet group (*, p<0.01, stearate VS. control diet group). Although mice on corn oil and safflower oil diet had lower number of metastasis, no significance was reached. (B) In experiment 2, two-way ANOVA showed that both paclitaxel therapy and diet stearate significantly reduced the number of lung metastases (PTX VS. no PTX group, p<0.01; stearate VS. control diet group, p<0.05). Although the number of metastasis was also decreased in corn oil diet group, no significance was reached.

FIG. 20. Diet therapy and chemotherapy on the size of lung metastasis. The size of lung metastatic tumors was measured with dissecting microscope (diameter <0.1 cm, small size; 0.1-0.2 cm, medium size; >0.2 cm, large size). The number of tumors of different size was counted and compared. (A) In experiment 1, mice on safflower oil and stearate diet had fewer small size lung metastasis (*, p<0.01, safflower or stearate VS. control diet group). (B) In experiment 2, mice on corn oil diet plus PTX and stearate diet plus PTX had significantly decreased number of medium and large size lung metastasis. The number of small size tumor was also significantly decreased in stearate diet and stearate diet plus PTX groups. (*, p<0.05, compared to control diet group; **, p<0.01, compared to control diet group)

FIG. 21. Diet therapy and chemotherapy on angiogenesis. Paraffin sections were prepared from lung metastatic tumors, and followed by CD31 immunostaining. A-F are representatives of CD31 staining from different experimental groups. Tumors from the stearate diet groups and chemotherapy groups have significantly reduced number of microvessels. (G) When microvessel density (MVD) was measured and compared, two-way ANOVA showed that both diet and chemotherapy decreased the MVD significantly. (PTX VS. no PTX group, p<0.01; stearate VS. control diet group, p<0.01; corn oil VS. control diet group, p<0.05). Further analysis showed that in the presence of chemotherapy, the effect of stearate was significantly decreased (*, p<0.01, stearate diet plus PTX VS. control diet plus PTX). In the absence of chemotherapy, although the MVD was decreased in both stearate and corn oil diet groups, no significant difference was observed.

FIG. 22. Diet therapy and chemotherapy on proliferation. Ki67 immunostaining was performed on lung metastatic tumor paraffin sections. (A-F) are representatives of Ki67 staining from different experimental groups. Obviously, tumors from the chemotherapy groups have significantly reduced number of Ki67 positive cells. (G) When the number and percentage of Ki67 positive cells were counted and calculated, two-way ANOVA analysis showed that chemotherapy significantly inhibited the proliferation (PTX VS. no PTX, p<0.01).

FIG. 23. Diet therapy and chemotherapy on apoptosis. Caspase-3 immunostaining was performed on metastatic tumor paraffin sections. (A-F) are representatives of caspase-3 staining from different experimental groups. Obviously, the tumor from the control diet group has the least number of caspase-3 positive cells. (G) When the number and percentage of caspase-3 positive cells were counted and calculated, tumors from stearate and corn oil diet groups had more caspase-3 positive cells (*, p<0.01, corn oil VS. control diet group; #, p<0.05, stearate VS. control diet group); however, in the presence of chemotherapy, this difference is not obvious. Tumors from control diet plus PTX group had significantly increased caspase-3 positive is cells compared to control diet group (&, p<0.05).

FIG. 24: A diagrammatic portrayal of the some of the known structure/activity relationships in taxanes.

FIG. 25: Effect of fatty acids on the expression of cIAP2, BAX, and Bcl-2.

### BEST MODE FOR CARRYING OUT INVENTION

### A. Definitions

With reference to the use of the word(s) “comprise” or “comprises” or “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The terms “prevention”, “prevent”, “preventing”, “suppression”, “suppress” and “suppressing” as used herein refer to a course of action (such as administering a compound or pharmaceutical composition of the present disclosure) initiated prior to the onset of a clinical manifestation of a disease state or condition so as to reduce the likelihood and/or severity of a clinical manifestation of the disease state or condition. Such preventing and suppressing need not be absolute to be useful. These terms are not meant to be construed to require the complete suppression of any sign or symptom of the disease state or condition.

The terms “treatment”, “treat” and “treating” as used herein refers a course of action (such as administering a compound or pharmaceutical composition) initiated after the onset of a clinical manifestation of a disease state or condition so as to eliminate or reduce the severity of such clinical manifestation of the disease state or condition. Such treating need not be absolute to be useful. These terms are to not meant to be construed to require the complete suppression of any sign or symptom of the disease state or condition.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method, compound or pharmaceutical composition of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as a part of a pharmaceutical composition, that is capable of having any detectable, positive effect on any symptom, aspect, or characteristic of a disease state or condition. Such effect need not be absolute to be beneficial.

The term “prodrug” as used herein includes functional derivatives of a disclosed compound which are readily convertible in vivo into the required 5 compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional to procedures for the selection and preparation of suitable prodrug derivatives are described, for example, in “Design of Prodrugs”, ed. H. Bundgaard, Elsevier, 1985.

The term “pharmaceutically acceptable salts” as used herein includes salts of the active compounds which are prepared with relatively nontoxic acids or bases, depending on the particular substituent found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “about” as used herein refers to an approximate range around a central value. The range encompasses the likely margin of error that would be encountered by one of ordinary skill in the art in attempting to make a measurement of the value.

### B. Compositions

As stated above, it has been unexpectedly discovered that dietary stearate is a potent agent for the treatment and prevention of diseases, specifically those related to fat and sugar metabolism, and cancer.

Stearate selectively reduces visceral fat content in animals, without affecting the animal's overall fat content or body weight. Stearate also selectively induces apoptosis of visceral preadipocytes without affecting mature adipocytes in vitro. Without wishing to be bound by any single hypothetical model, it is possible that the apoptotic effect of stearate is the cause of the reduction in the mass visceral adipose tissue. The reduction of visceral fat content could in turn result in many health benefits, such as the prevention of cardiovascular disease and cancer (both of which are associated with high visceral fat content).

Previously it was believed that dietary stearate could contribute to obesity and insulin resistance. Contrary to these beliefs, it has been unexpectedly observed that dietary stearate reduces blood glucose and leptin concentrations in vivo, without any pathological effects on the liver or the kidneys.

It has also been unexpectedly discovered that stearate inhibits the cell cycle progression of tumor cells both in vivo and in vitro. Tumor cells exposed to stearate in vitro showed inhibited cell cycle progression at both the G₁ and G₂ phases. Stearate increases the expression of p21^{CIP1/WAF1} and p27^{KIP1}, both of which are cell-cycle inhibitors. Stearate increases the binding of Ras to GTP in vitro. Stearate was also discovered to inhibit the phosphorylation of Cdk2. Without wishing to be bound by any particular hypothetical model, it is possible that stearate inhibits Cdk2 phosphorylation through the increased expression of p21^{KIP1}, which is an inhibitor of Cdk2 phosphorylation. Furthermore, stearate has now been observed to increase Rho activation and expression in vitro; however, in cells constitutively expressing RhoC, stearate did not increase expression of p21^{KIP1}.

Stearate also has a positive effect on the incidence, number, and size of mammary tumors in vivo. Without wishing to be bound by any hypothetical model, this may be due to stearate's ability to arrest cell cycle progression in tumors through increased expression of p21^{CIP1/WAF1} and p27^{KIP1}. Biopsies of the tumors revealed decreased expression of RhoA, RhoC, and total Rho. It would thus appear that stearate simultaneously inhibits Rho while activating Ras.

Furthermore, when used in conjunction with chemotherapeutic agents, stearate reduces the incidence and severity of cancer in vivo if administered either before or after carcinogenesis. The inhibitory effect of stearate and paclitaxel on tumor number and mass greatly exceeds that of either stearate alone or paclitaxel alone.

1. Stearate Compounds

The compositions provided in this disclosure provide stearate compounds. In a general embodiment the stearate compound is any stearate compound suitable for the intended purpose of the composition. For example, compositions to be administered to a subject in vivo (such as pharmaceutical compositions, dietary supplements, and food items) may be selected on the basis of any of toxicity, absorption characteristics, stability during ingestion, palatability, and storability. Compositions to which cells are to be exposed in vitro may be selected on the basis of any of cytotoxicity, solubility, pKₐ, and effect on osmolarity.

Some embodiments of the stearate compound exclude at least one of a naturally occurring stearate compound, a phospholipid stearate compound, a stearoyl triglyceride, a stearoyl ester, a naturally occurring phospholipid stearate compound, a naturally occurring stearoyl triglyceride, and a naturally occurring stearoyl ester. In compositions intended to be eaten or taken orally the stearate compound may be an edible stearate compound, being essentially nontoxic and capable of absorption in the gastrointestinal tract. The stearate compound may be a salt, such as an edible salt (for example in the case of a food item or a dietary supplement) or a pharmaceutically acceptable salt (in the case of a pharmaceutical composition).

The stearate compound may be stearic acid. Stearic acid has the advantages of being commercially available, inexpensive, and well characterized toxicologically. Alternatively, the stearate compound may be a phospholipid stearate compound, a stearoyl triglyceride, or a stearoyl ester.

The stearate compound may be present in an amount sufficient to achieve an effect that is the purpose of the composition. Such an effect may be one or more of: reducing visceral fat content, reducing total body fat content, reducing the likelihood or severity of cardiovascular disease, reducing the likelihood or severity of tumorigenesis, reducing the likelihood or severity of metastasis, reducing serum glucose concentration, reducing leptin concentration, increasing serum MCP-1, and reducing the likelihood or severity of type 2 diabetes. In some embodiments the amount of stearate will be an amount sufficient to treat and/or prevent a disease state or condition, such as any of those listed above.

In some instances the amount of the stearate compound will be sufficient to achieve a specified cellular effect. For example, the stearate compound may be present in an amount effective to reduce the activity in a subject of at least one of RhoA, Rho C, and total Rho. In some embodiments the stearate compound is present in an amount effective to at least partially arrest at G1 the cell cycle of a tumor cell in a subject. In some embodiments the stearate compound is present in an amount effective to increase Ras activity in a subject, increase ERK. phosphorylation in a subject, increase p21^{CIP1/WAF1} activity in a subject, increase p27^{KIP1} activity in a subject, or a combination of the foregoing. The amount of the stearate compound may be sufficient to achieve a target extracellular concentration. Such amounts can be determined by those of ordinary skill in the art on the basis of established pharmacokinetic models. In a particular embodiment, the effective amount is an amount adequate to achieve an extracellular concentration of the stearate compound of about 50 μM.

2. Dietary Supplements and Food Items

The disclosure provides a dietary supplement and a food item comprising a substantial amount of a stearate compound. The stearate compound may be any that is disclosed as suitable in the preceding section. In certain embodiments, the stearate compound is neither a naturally occurring triglyceride compound nor a naturally occurring phospholipid compound. In some embodiments the stearate compound is neither a triglyceride nor a phospholipid compound. In some embodiments the stearate compound is stearic acid or an edible salt thereof. In further embodiments, the stearate compound is not a stearate ester. In a particular embodiment, the stearate compound is stearic acid.

The food item comprises a food, an edible and desirable substance of biological origin, countless varieties of which are known in the art. Embodiments of the food item may include a ready-to-eat processed food item, such as a juice-based drink, a shake, a wafer, a candy, a tea, a sauce, an edible oil, a spread, and a baked product. The food item may also be less processed. Some embodiments of the food item are processed to remove at least a portion of the naturally occurring lipid in the food item, which is at least partially replaced with the stearate compound. In other embodiments the food item is enriched in the stearate compound without other modification of the original lipid content. The food item allows the subject to consume a substantial amount of stearate pleasantly with a snack or meal, without the need for large dosage forms such as capsules.

The food item may further comprise one or more food additives. Food additives are substances that are not naturally found in the food, but are added to confer desirable properties. They include anti-caking agents, antifoaming agents, defoaming agents, antioxidants, boiler compounds, bleaching agents, flour-maturing agents, buffer and neutralizing agents, components or coatings for fruits and vegetables, dietary supplements, emulsifiers, enzymes, essential oils, oleoresins, natural flavoring agents, substance used in conjunction with flavors, fumigants, fungicides, herbicides, hormones, inhibitors, natural substances and extractives, non-nutritive sweeteners, nutrients, nutritive sweeteners, pesticides other than fumigants, chemical preservatives, sanitizing agents for food processing equipment, solubilizing and dispersing agents, sequestrants, solvents, spices, other natural seasonings and flavorings, spray adjuvant, stabilizers, synthetic flavors, and veterinary medicine residue. One of ordinary skill in the art will understand which types of food additives are appropriate for a given type of food. The food additives may be selected from the list maintained by the United States Food and Drug Administration of additives considered to be safe for human consumption under approved conditions, which is incorporated herein by reference only for this teaching (see http://www.fda.gov/Food/FoodIngredientsPackaging/FoodAdditives/FoodAdditiveListings/ucm091048.htm).

The dietary supplement is an oral formulation of the stearate compound. The formulation will be in an oral dosage form, such as but not limited to, tablets, capsules, sachets, lozenges, troches, pills, powders, or granules. The stearate compound may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, a stearate lubricant (such as magnesium stearate, calcium stearate, zinc stearate, stearic acid, etc.) as well as the other carriers described herein. Lozenge forms can comprise the active ingredient in a flavor, for example sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, the stearate compounds of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable coloring agents or other accessory agents can also be incorporated into the mixture. Other dispersing agents that may be employed include glycerin and the like.

Additional ingredients may be added to the dietary supplement, such as those that are described below as suitable for oral dosage forms in pharmaceutical compositions.

In some embodiments of the dietary supplement and the food item the stearate compound is present in at least 2% by weight. In further embodiments, the stearate compound is present in at least 17% by weight. In yet further embodiments the stearate compound is present in at least 90% by weight, or about 100% (this might include food items such as cooking oil or butter substitutes, or dietary supplements). In yet further embodiments, the amount of stearate is sufficient to achieve a target amount of total daily intake of the stearate compound. This may be a fraction of the subject's total recommended fat intake; the fraction may be selected from the group consisting of: 10, 20, 30, 40, 50, 60, 70, 80, 90 or 100%. Alternatively, the fraction may be at least a fraction of the subject's total recommended saturated fat intake; such as at least 10, 20, 30, 40, 50, 60, 70, 80, 90 or 100%. The target amount may also be a range bounded by any two of the foregoing fractions. The amount may also be a fraction of the subject's recommended fat intake, less the subject's minimum required intake of essential fatty acids. Recommended intake of fats, essentially fatty acids, and saturated fats are generally ascertained based on the subject's sex, height, and level of activity. Those of ordinary skill in the art can determine a subject's recommended intake of such lipids without undue experimentation. For example, various medical organizations and governmental agencies provide easy methods of calculating these values to enable members of the public to make informed dietary decisions.

3. Pharmaceutical Compositions

A pharmaceutical preparation is provided, comprising a therapeutically effective amount of a stearate compound. The therapeutically effective amount may be sufficient to have a detectable, positive effect on any symptom, aspect, or characteristics of a disease state or condition listed above. The compositions disclosed may comprise one or more stearate compound, in combination with a pharmaceutically acceptable carrier. Examples of such carriers and methods of formulation may be found in Remington: The Science and Practice of Pharmacy (20th Ed., Lippincott, Williams & Wilkins, Daniel Limmer, editor). To form a pharmaceutically acceptable composition suitable for administration, such compositions will contain a therapeutically effective amount of a compound(s).

The pharmaceutical compositions of the disclosure may be used in the treatment and prevention methods of the present disclosure. Such compositions are administered to a subject in amounts sufficient to deliver a therapeutically effective is amount of the compound(s) so as to be effective in the methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. Exemplary routes of administration include, but are not limited to, subcutaneous, intravenous, topical, epicutaneous, oral, intraosseous, intramuscular, intranasal and pulmonary. In some embodiments, the therapeutically effective amount or effective inhibitory amount will be sufficient to achieve an extracellular concentration of the compound at or about 50 μM.

The compositions of the present disclosure may be administered only one time to the subject or more than one time to the subject. Furthermore, when the compositions are administered to the subject more than once, a variety of regimens may be used, such as, but not limited to, once per meal, once per day, once per week, once per month, or once per year. The compositions may also be administered to the subject more than one time per day. The therapeutically effective amount of the molecules and appropriate dosing regimens may be identified by routine testing in order to obtain optimal activity, while minimizing any potential side effects.

In addition, co-administration or sequential administration of complementary agents may be desirable. The complementary agent may be, for example, an antineoplastic agent. Some embodiments of the antineoplastic agents act through mechanisms other than a diacylglycerol/protein kinase C dependent pathway; without wishing to be bound by any given hypothetical model, stearate may act through this pathway, and so antineoplastic agents that act through another pathway would be expected to complement stearate. Exemplary embodiments of to such antineoplastic agents include alkylating agents (e.g. cyclophosphamide, which act directly on DNA), taxanes and vinca alkaloids (which disrupt microtubules), 5 fluorouracil (a thymidylate synthase inhibitor), antimetabolites (which block DNA synthesis), topoisomerase inhibitors (which inhibit DNA production and replication), and cytotoxic antibiotics such as doxorubicin and bleomycin (which act directly on DNA). One embodiment of the antineoplastic complementary agent is a taxane compound. The taxane compound may be any known in the art, for example paclitaxel (TAXOL) and docetaxel. In a particular embodiment the antineoplastic complementary agent is paclitaxel. The amount of taxane will be an amount that is considered safe and effective, as are known to those of ordinary skill in the art. Taxane compounds have the advantages of being effective and well-tolerated antineoplastic agents which complement stearate.

The compositions of the present disclosure may be administered systemically, such as by intravenous administration, or locally such as by subcutaneous injection or by application of a paste or cream. In a particular embodiment the pharmaceutical composition is administered orally.

The compositions of the present disclosure may further comprise agents which improve the solubility, half-life, absorption, etc. of the compound(s). Furthermore, the compositions of the present disclosure may further comprise agents that attenuate undesirable side effects and/or or decrease the toxicity of the compounds(s). Examples of such agents are described in a variety of texts, such as, but not limited to, Remington: The Science and Practice of Pharmacy (20th Ed., Lippincott, Williams & Wilkins, Daniel Limmer, editor).

The compositions of the present disclosure can be administered in a wide variety of dosage forms for administration. For example, the compositions can be administered in forms, such as, but not limited to, tablets, capsules, sachets, lozenges, troches, pills, powders, granules, tinctures, solutions, suspensions, elixirs, syrups, ointments, creams, pastes, emulsions, or solutions for intravenous administration or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the compound(s) may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, the molecules of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include: (a) cationic detergents such as, for example, dimethyldialkylammonium halides, and alkylpyridinium halides; (b) anionic detergents such as, for example, alkyl, aryl, and olefin sulfonates, alkyl, olefin, ether, and monoglyceride sulfates, and sulfosuccinates; (c) nonionic detergents such as, for example, fatty amine oxides, fatty acid alkanolamides, and polyoxyethylene polypropylene copolymers; (d) amphoteric detergents such as, for example, alkylbeta-aminopropionates, and 2-alkylimidazoline quaternary ammonium salts; and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the molecule of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The compound(s) of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain monoclonal antibodies to direct delivery of the liposome to a particular cell type or group of cell types.

The compound(s) of the present disclosure may also be coupled with soluble polymers as targetable drug carriers. Such polymers can include, but are not limited to, polyvinyl-pyrrolidone, pyran copolymer, polyhydroxypropylmethacrylamidephenol, polyhydroxyethylaspartamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### C. Methods

A method of improving or maintaining the health of a subject is provided, the method comprising administering to the subject an effective amount of a stearate compound. In some embodiments of the methods the stearate compound is not a naturally occurring triglyceride compound or a naturally occurring phospholipid compound. The stearate compound may be administered in the form of any of the compositions described above. The health of the subject may be improved or maintained by controlling the visceral fat content of the subject, reducing the likelihood or severity of tumorigenesis in the subject, controlling the total body fat content of the subject, reducing the likelihood or severity of cardiovascular disease in the subject, or reducing the likelihood or severity of type-2 diabetes in the subject.

Embodiments of the method comprise treating or preventing a disease state or condition, such as cardiovascular disease, primary tumorigenesis, metastasis, type-2 diabetes, obesity, or conditions and disease states associated with the foregoing. In further embodiments of the method, the method comprises identifying a subject in need of treatment or prevention of the condition or disease state.

The compositions of the present disclosure may be administered only one time to the subject or more than one time to the subject. Furthermore, when the compositions are administered to the subject more than once, a variety of regimens may be used, such as, but not limited to, once per meal, once per day, once per week, once per month, or once per year. The compositions may also be administered to the subject more than one time per day. The therapeutically effective amount of the molecules and appropriate dosing regimens may be identified by routine testing in order to obtain optimal activity, while minimizing any potential side effects.

A method of inhibiting the cell cycle progression of a cell is provided, said method comprising contacting the cell with an inhibitory effective amount of a stearate compound. In an exemplary embodiment of the method the effective amount is about 50 μM.

In various embodiments of the method the amount of the stearate compound is effective to have one or more specified effects on the cell. In some embodiments the amount is effective to increase at least one of Ras activity, ERK phosphorylation, p21^{CIP1/WAF1} activity, or p27^{KIP1} activity. In further embodiments the amount is effective to reduce the activity of at least one of RhoA, Rho C, and total Rho. In a specific embodiment the cell is a tumor cell, and the amount is effective to at least partially arrest the cell cycle of the tumor cell at G1. In an exemplary embodiment of the method the effective amount is about 50 μM.

A method of inducing apoptosis in a visceral pre-adipocyte cell is provided, comprising contacting the cell with an effective amount of a stearate compound, other than a naturally occurring triglyceride compound or a naturally occurring phospholipid compound. In an exemplary embodiment of the method the effective amount is about 50 μM.

In addition, in the above methods in which the goal is to arrest the cell cycle or control tumorigenesis, it may be desirable to administer a complementary agent. The complementary agent may be, for example, an antineoplastic agent. One embodiment of the antineoplastic complementary agent is a taxane compound. The taxane compound may be any known in the art, for example paclitaxel (TAXOL) and docetaxel. In a particular embodiment the antineoplastic complementary agent is paclitaxel. The amount of taxane will be an amount that is considered safe and effective, as are known to those of ordinary skill in the art.

The antineoplastic agent may be taxane or a taxane derivative. Taxane derivatives comprise a common taxane skeleton, as shown below:

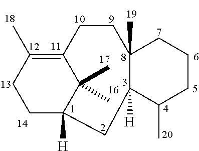

Some embodiments of the antineoplastic agent are taxane derivatives with antineoplastic activity. Two currently approved taxane derivatives, paclitaxel and docetaxel, comprise the skeleton as shown below:

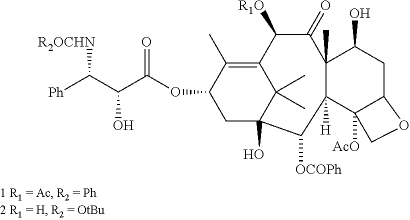

The R substitutions in line 1 are paclitaxel and the R substitutions in line 2 are docetaxel.

Other taxane derivatives are known in the art, and are too numerous to list and describe within this disclosure. Examples of U.S. Patent documents that describe taxane derivatives with antineoplastic activity include U.S. Pat. No. 6,268,381, U.S. Pat. No. 5,912,263, U.S. Pat. No. 5,580,899, U.S. Pat. No. 5,646,176, U.S. Pat. No. 5,698,582, U.S. Pat. No. 4,814,470, US Pat. Pub. 2002/0002292, U.S. Pat. No. 5,817,840, U.S. Pat. No. 5,821,263, U.S. Pat. No. 6,147,234, U.S. Pat. No. 6,191,290, U.S. Pat. No. 5,703,117, U.S. Pat. No. 5,476,954, U.S. Pat. Pub. 2010/0168420, and U.S. Pat. No. 6,339,164 (all of the foregoing are incorporated herein by reference only to teach these taxane derivatives).

One of ordinary skill in the art can determine which taxane derivatives possess antineoplastic activity by applying known relationships between the structure of taxane derivatives and their antineoplastic activity. Taxanes function by stabilizing microtubules. Unlike previous antineoplastic agents that act on tubulin (such as Catharanthus alkaloids), taxanes induce the assembly of tubulin and inhibits its disassembly. By this mechanism it is believed that taxanes arrest mitosis by stabilizing microtubules.

The relationship between the structure and function of taxane derivatives has been the subject of numerous studies and several scholarly reviews available to those of ordinary skill in the art. The relationship between structure and function was reviewed, for example, by Guéritte, Current Pharmaceutical Design 7:1229-1249 (2001); and Kingston et al., Curr Opin Drug Discov Devel. 10(2):130-44 (2007). The taxane binding sites of tubulin have been described, for example, by Löwe at al., J. Molecular Biology 313:1045-1057 (2001). The effects of taxane conformation on tubulin binding have also been described, for example, by Snyder et al., PNAS 98(9): 5312-5316 (2001). The foregoing articles are incorporated herein by reference to allow one of ordinary skill in the art discern antineoplastic taxane derivatives based on the structure of the taxane derivative.

It has been observed that the “northern” region of the taxane skeleton (C₇, C₉, and C₁₀) can be altered without loss of activity. Alteration of the northern region can affect delivery, stability, and solubility of the molecule. For example, C₉ and C₁₀ deoxy taxane derivatives have similar activity to paclitaxel, as have C₉ and C₁₀ alkyl taxane derivatives and amino taxane derivatives. Longer alkyl groups at these positions reduce activity by decreasing interaction with tubulin (polar groups at these positions increase interaction with tubulin).

The isoserine side chain at C₁₃ is critical for activity. Within this chain, the 2′ hydroxyl group must be preserved, although it may be substituted with an ester (or other compound that readily converts to a hydroxyl group). Modifications of 3′ group and the N-3′ group can be made while preserving activity and in some cases will increase it. Taxane derivatives have been observed to retain activity in the presence of an acyl, aroyl, carbonate, and other hydrophobic groups at C₂ and C₄, which are considered to be critical for activity. In contrast, the moieties bound to C₁ and C₁₄ appear to be less critical.

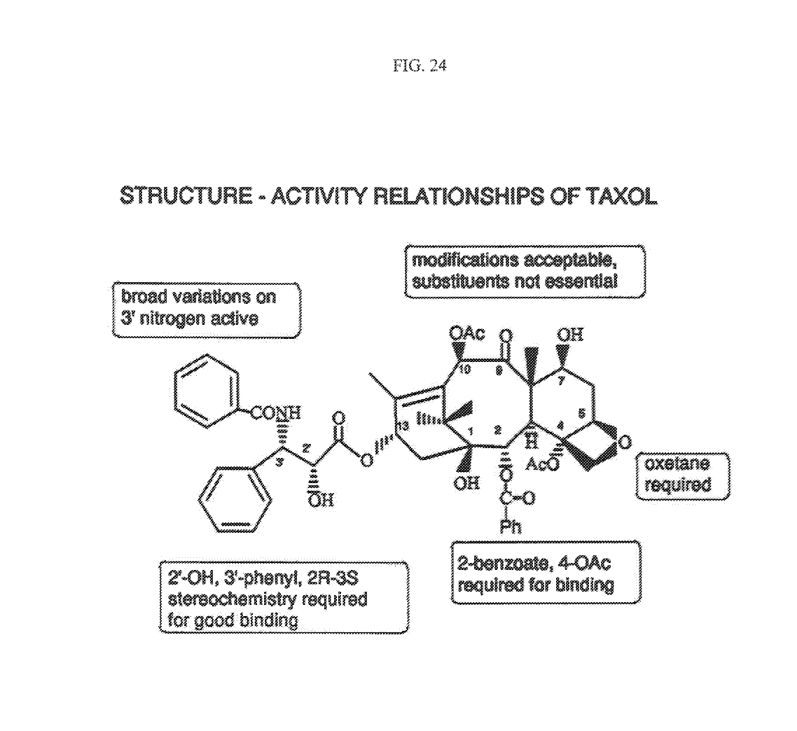

Some of the understood structure/function relationships in the taxane skeleton are shown in FIG. 24.

Some embodiments of the taxane derivative are of the general formula (I) shown below:

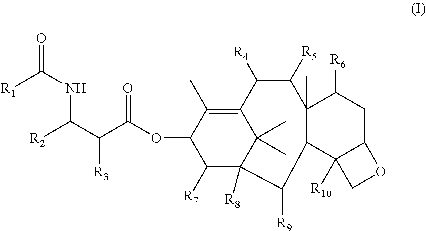

wherein:
R₁, R₂, and R₄-R₈ are unrestricted;
R₃ is hydroxyl or ester;
R₉ is acyl, aroyl, carbonate, or alkyl; and
R₁₀ is acyl, aroyl, carbonate, or alkyl.

### D. Examples

### 1. Working Example

### Selective Reduction of Visceral Fat

### Abstract

The effect of dietary stearate on body fat accumulation was evaluated. Athymic nude mice were fed with a stearate enriched diet for 18 weeks and compared with mice fed diets enriched in linoleate (safflower oil), oleate (corn oil), and low fat diet mouse chow as a further control under identical conditions. Total body fat (TBF) was measured by dual energy X-ray absorptiometry (DXA) and quantitative magnetic resonance (QMR), the abdominal fat and other organs were weighed, and selective serum parameters were measured including glucose, insulin, and related inflammatory markers. Abdominal fat was reduced by 70% in the stearate fed group, while total body fat was only slightly but significantly reduced when measured by DXA. Correspondingly, lean body mass was slightly but significantly increased. There was no difference in the weight of brain, heart, lungs or liver although stearate diet mice had slightly reduced kidney weights. Stearate significantly reduced serum glucose compared to all other diets and increased MCP-1 compared to the low fat control. The low fat control diet had increased serum leptin compared to all other diets. In vitro studies using 3T3L1 cells were subsequently used to determine the direct effects of stearate on fat cell differentiation, preadipocytes and on differentiated adipocytes. Stearate had no direct effects on the process of differentiation or on mature adipocytes. However stearate did cause cytotoxicity and apoptosis in preadipocytes and the apoptosis was at least in part characterized by increased caspase-3 activity. CONCLUSION: Dietary stearate dramatically and selectively reduces visceral fat due in part to causing apoptosis of preadipocytes. Reduction in visceral fat and serum glucose by dietary stearate may be related to the previously recognized beneficial effects of it on breast cancer proliferation and possibly in other obesity associated diseases.

### Introduction

Stearate, an 18-carbon long chain saturated fatty acid (SFA), is found in high concentrations in many foods in the Western diet including beef, chocolate, and milk fats. Although stearate shares many physical properties with the other long-chain SFA, such as palmitate (C 16:0), it has different physiological effects. Unlike palmitate, stearate does not raise serum cholesterol (TC) or LDL-cholesterol (LDL-C)^{(1,2)} Therefore, stearate has been proposed as a substitute for cholesterol-raising SFA and trans fatty acids in food manufacturing^{(1,2)}. Its unique anti-breast cancer properties^{(3,4,5)} suggest a possible use of dietary stearate in cancer prevention and treatment. Obesity is known to be a risk factor for breast cancer initiation and progression. In addition, obese breast cancer patients are known to have worse outcomes than non-obese breast cancer patients. Worse outcomes are directly linked to metastasis, the cause of death for most cancer patients. Previous studies have investigated the role of dietary fat on obesity, and the results indicate that dietary fat per se is surprisingly not directly thought to cause obesity. Other studies have attempted to investigate individual fatty acids. However these studies are difficult to interpret since mixtures of fatty acids were used in practice.

In this study, it was investigated whether dietary stearate affects body fat accumulation in vivo and possible mechanisms in vitro. A stearate diet was used that contained the minimum amount of essential fatty acid required for normal growth and development, and added to that dietary stearate. This diet minimizes the confounding effects of other fatty acids while not affecting total body weight.

### Materials and Methods

Reagents

Stearate, oleate, linoleate, diatomaceous earth, insulin, dexamethasone, 3-isobutyl-1-methyl-xanthine, and fatty acid free BSA were obtained from the Sigma-Aldrich Chemical Co. (St. Louis, Mo.). EnzCheck Capase-3 Activity Kit #1, TrypLE™ Express stable trypsin-like enzyme and the Dead Cell Apoptosis Kit with Annexin V Alexa Fluor® 488 and propidium iodide (PI) were purchased from Invitrogen (Carlsbad, Calif.). A NEFA C kit was obtained from Wako Chemicals (Richmond, Va.). A cytotoxicity detection kit was obtained from Roche Molecular Biological Co. (Indianapolis, Ind.). Trypan blue was purchased from Eastman Kodak Company (Rochester, N.Y.). Oil Red O was acquired from Rowley Biochemical (Rowley, Mass.) and Hematoxylin I was obtained from Richard-Allan Scientific (Kalamazoo, Mich.).

Animals and Diets

Three-to-four week old female athymic mice were purchased from Harlan Sprague Dawley, Inc. (Indianapolis, Ind.) and were maintained in microisolater cages in pathogen-free facilities. Animals were divided randomly into four groups, and were placed on one of four diets: a low fat diet (5% corn oil diet) comparable to normal rodent chow, a 20% safflower oil diet, a 17% corn oil/3% safflower oil diet and a 17% stearate/3% safflower oil diet. The diets were prepared by Harlan-Teklad (Madison, Wis.). The animals were fed ad libitum for 18 weeks and the amount of food consumed was recorded. Mice were anesthetized with 3% isoflurane in 2.5% O₂ and weighed weekly. At the end of the experiment, the mice were sacrificed, the brain, heart, lungs, kidneys, liver, and abdominal fat were collected. All in vivo procedures were approved by the Institutional Animal Care and Use Committee (IACUC), University of Alabama at Birmingham (UAB).

Dual Energy X-Ray Absorptiometry (DXA)

Mice were scanned using the GE Lunar PIXImus dual-energy X-ray absorptiometer (DXA) with software version 1.45 after 18 weeks on their respective diets. Each animal was placed in an airtight container and anesthetized using the microdrop method with Isoflurane (4%). Once the mouse was immobile and breathing steadily, it was placed in a prostrate position on the DXA imaging plate and scanned. During the scan the mouse remained anesthetized using an Isoflurane (3%) and oxygen (500 ml/min) mixture delivered by a Surgivet anesthesia machine. Each scan took less than 5 minutes. Data obtained from these scans included bone mineral content (BMC), bone mineral density (BMD), lean mass and fat mass.

Quantitative Magnetic Resonance (QMR)

In vivo body composition (total body fat and lean tissue) of mice was determined using an EchoMRI 3-in-1 quantitative magnetic resonance (QMR) instrument (Echo Medical Systems, Houston, Tex.). Each animal was placed in a clear tube and the tube was capped with a stopper that restricted vertical movement, but allowed constant airflow. No anesthesia was required. The tube was inserted into the instrument and scanning was initiated. Once scanning was complete (less than 2 minutes) the animal was returned to its home cage. This procedure provided data on fat and lean mass.

Measurement of Serum Glucose, Insulin, Leptin, MCP-1, IL-6, and Adiponectin.

IL-6 and MCP-1 was analyzed in mouse serum using Meso Scale Discovery (Gaithersburg, Md.) mouse cytokine assay ultra-sensitive kits. The coefficient of variation (CV) for these assays was 9% and 3% respectively. Mouse serum leptin, insulin and adiponectin were measured using Millipore (Billerica, Mass.) radioimmunoassay kits with CVs of 7%, 4% and 2% respectively. Serum glucose was measured by a glucose oxidase assay run on a Stanbio Sirrus instrument (Stanbio Laboratory, Boerne, Tex.). This assay has a 3% CV.

Paraffin Section and H&E Staining

Paraffin sections were prepared as described previously (41). Briefly, 10% buffered formalin fixed samples (abdominal fat, kidney and liver) were processed with a VIP 1000 tissue processor (Sakura-Finetek, Torrance, Calif.) through graded alcohols and xylene, then embedded into paraffin blocks. Five micron sections were cut on a Leica 2135 rotary microtome (Leica Microsystems, Bannockburn, Ill.), air-dried, deparaffinized and stained with Hematoxylin & Eosin stains (Richard Allen Scientific, Kalamazoo, Mich.).

3T3L1 Cell Culture

3T3L1 mouse fibroblast cells (ATCC, CL-173™) were maintained according to the manufacture's protocol, in Dulbecco's modified eagle's medium (DMEM) containing 10% Fetal Bovine Serum and antibiotics (M1 medium). Adipocyte differentiation was performed according to standard procedures (42). Briefly, the 3T3L1 fibroblasts were seeded at 30% confluence and allowed to grow to near 100% confluence. On the day after reaching maximum confluence, conversion was induced by replacing the M1 medium with M1 medium containing insulin (5 μg/ml), dexamethasone (0.25 μM), and 3-isobutyl-1-methyl-xanthine (0.5 mM). After 2 days, the cells were changed to M1 medium with insulin (5 μg/ml) for an additional 2 days. Thereafter, the cells were maintained in M1 medium without additives for 2 days.

Fatty Acids

50 μM of stearate, oleate or linoleate was used to treat 3T3L1 cells as this concentration is centered within the normal range for non-esterified stearate in the plasma of humans. Stearate, oleate, or linoleate was loaded onto fatty acid free BSA according to the method reported by Spector and Hoak (Spector and Hoak, 1969). Briefly, stearate (0.5 g) was dissolved in chloroform (100 mL) and mixed well with 10 g diatomaceous earth in a 1 liter flask. The mixture was stirred and dried under nitrogen until powder. Fatty acid free BSA (1 g) was dissolved in 100 mL with DMEM without phenol red and mixed with 3 g of the stearate/diatomaceous earth mixture and stirred for 30 minutes. The stearate/BSA solution was filtered through a 0.45 μm filter, and adjusted to pH 7.4. The concentration of stearate in the solution was detected by use of a NEFA C kit. Oleic and linoleate were loaded in the same way. All experimental data on 3T3L1 cells were confirmed using fatty acid free BSA control solutions that were put through the same preparatory procedure described except for the fact that no fatty acid was added. Before the treatment with fatty acid, 3T3L1 preadipocytes were grown to 100%, and were starved in 2% FBS medium for 24 hours.

Flow Cytometry Analysis

After treatment, 3T3L1 cells were harvested, washed with cold PBS, and then resuspended in 100 μl annexin-binding buffer (50 mM HEPES, 700 mM NaCl, 12.5 mM CaCl₂, pH 7.4). Cell density was determined, and the cells were diluted to 10⁶ cells. Then 5μL of Alex Fluo 488 annexin V and 1 μL propidium iodide (PI) were added. Cells were gently oscillated and incubated for 15 min at room temperature. After adding 400 μL of binding buffer to each tube, cells were kept on ice and analyzed by flow cytometry within 1 hour. Cells that stained positive for Alex Fluo 488 annexin V and negative for PI were considered to be apoptotic. Cells that stained positive for both Alex Fluo 488 annexin V and PI were considered either in the end stage of apoptosis, or necrosis. Cells that stained negative for both Alex Fluo 488 annexin V and PI were considered alive and not undergoing measurable apoptosis. A BD LSR II flow cytometer from Becton Dickinson was used in all flow experiments and the data were analyzed with BD FACSDiva™ software V.6.1.3.

Cytotoxicity Assay

Lactate dehydrogenase (LDH) release was measured using a cytotoxicity detection kit, according to the manufacture's protocol. After 3T3L1 cells were treated, 1 ml of cell culture medium was removed and centrifuged. The supernatant was retained for assaying. For determinations, 100 μl of LDH assay reagent was added to 100 μl of supernatant and incubated for 30 min at room temperature in the dark. Absorbance was measured at 490 nm. Background release from culture medium alone was subtracted before reporting. Maximum release was measured after adding 2% Triton X-100 to untreated cells.

Trypan Blue Staining

After treatment, 3T3L1 cells were harvested and stained with 0.4% trypan blue solution. Cells in the four corners of the grid were counted under a conventional bright field binocular microscope.

Oil Red O Staining

Cellular lipids were stained with oil red O. Briefly, identical numbers of 3T3L1 cells were placed in 6-well plates, cultured and converted to adipocytes as described above. The cells were then fixed with 4% paraformaldehyde for 30 minutes and stained with a working solution of oil red O for 5 minutes. The cell nucleus was counterstained with hematoxylin. 200 cells were counted under the microscope in each sample, and the percentage of converted adipocytes was calculated. For OD measurement, cells were stained with oil red O in the same way. The oil red O was then eluted with 1 ml 100% isopropanol, and the OD was measured at 520 nm in a spectrophotometer.

Caspase-3 Activity Assay

Caspase-3 activity was measured using the EnzCheck Capase-3 Activity Kit #1 according to the manufacturer's instructions (Molecular Probes Inc. Eugene, Oreg.).

Statistical Analysis

Data were presented as the mean±standard error of the mean (SEM). Statistical comparisons were performed by one-way analysis of variance (ANOVA) using the SigmaStat 3.1® software program. A Holm-Sidak test was used where appropriate for step-down pairwise comparisons. Significant differences are set as p<0.05.

### Results

Diets, Food Intake and Weight

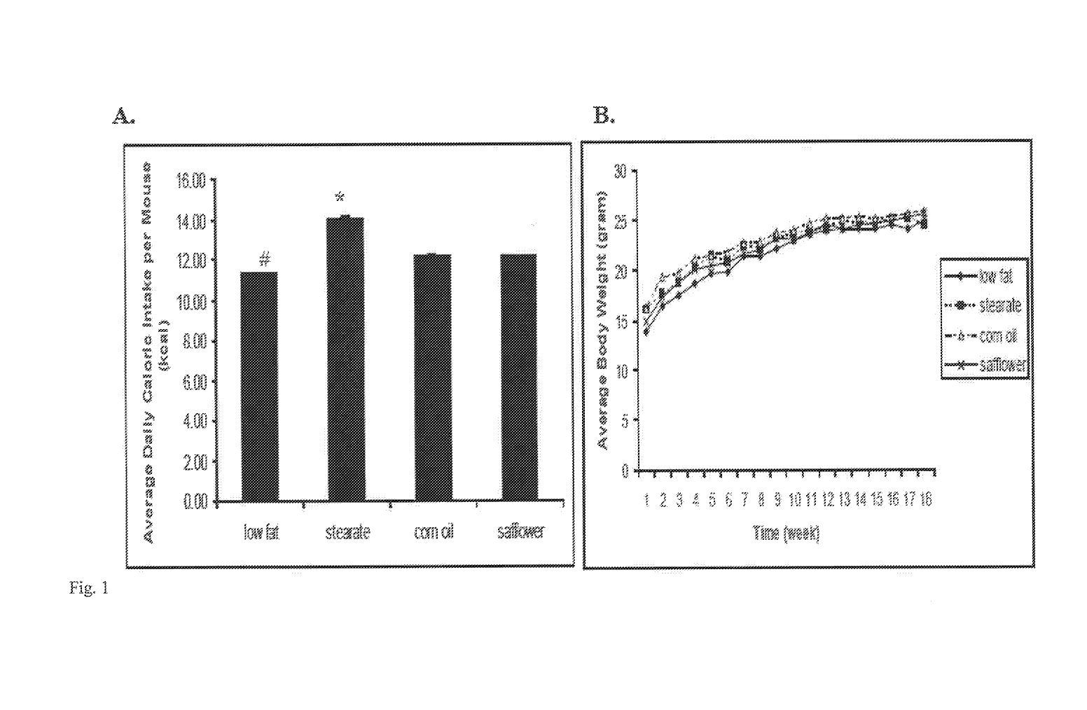

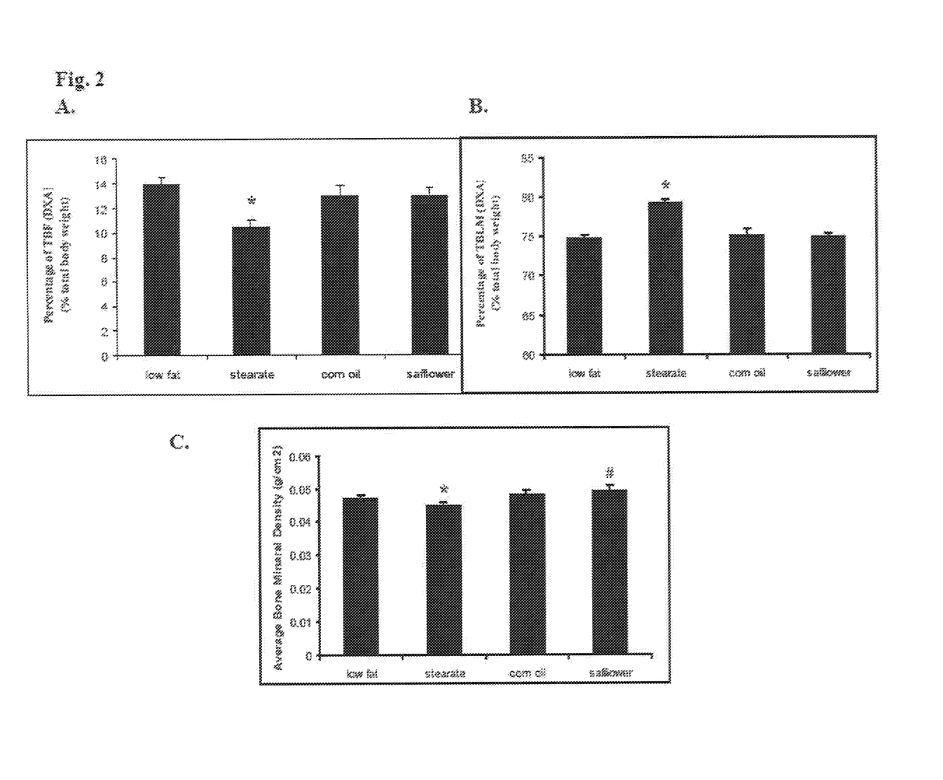

In order to determine possible changes in fat, lean mass and bone density, the mice were divided randomly into four groups, and placed on one of four diets—a low fat diet, a 20% safflower oil diet, 17% corn oil/3% safflower oil diet or a 17% stearate/3% safflower oil diet for 18 weeks. Because the four kinds of diets were not fully isocaloric, food and weight consumption were monitored again to ensure the animals did not have significant discrepancies in energy intake. As shown in FIG. 1 A, mice on the low fat diet consumed slightly less calories than mice on the other diets. Despite differences in food intake, there was no significant difference in weight gain between the diets (FIG. 1 B).

Dietary Stearate Reduces Both Abdominal Fat and Total Body Fat (TBF)

In order to determine global changes in TBF and bone mineral density (BMD), these parameters were checked by DEXA. The percentage of TBF decreased significantly (FIG. 2 A), while the percentage of total body lean mass (TBLM) increased significantly (FIG. 2 B) in the stearate diet group. The percentage change, however was small (<4%) indicating that only a small amount of TBF was lost. No significant changes were observed when TBF and TBLM were measured by QMR (data not shown).

Mice on the stearate diet had a significantly reduced BMD compared to all other experimental groups; while it was minimally elevated in the safflower oil group compared to the low fat control group (FIG. 2 C).

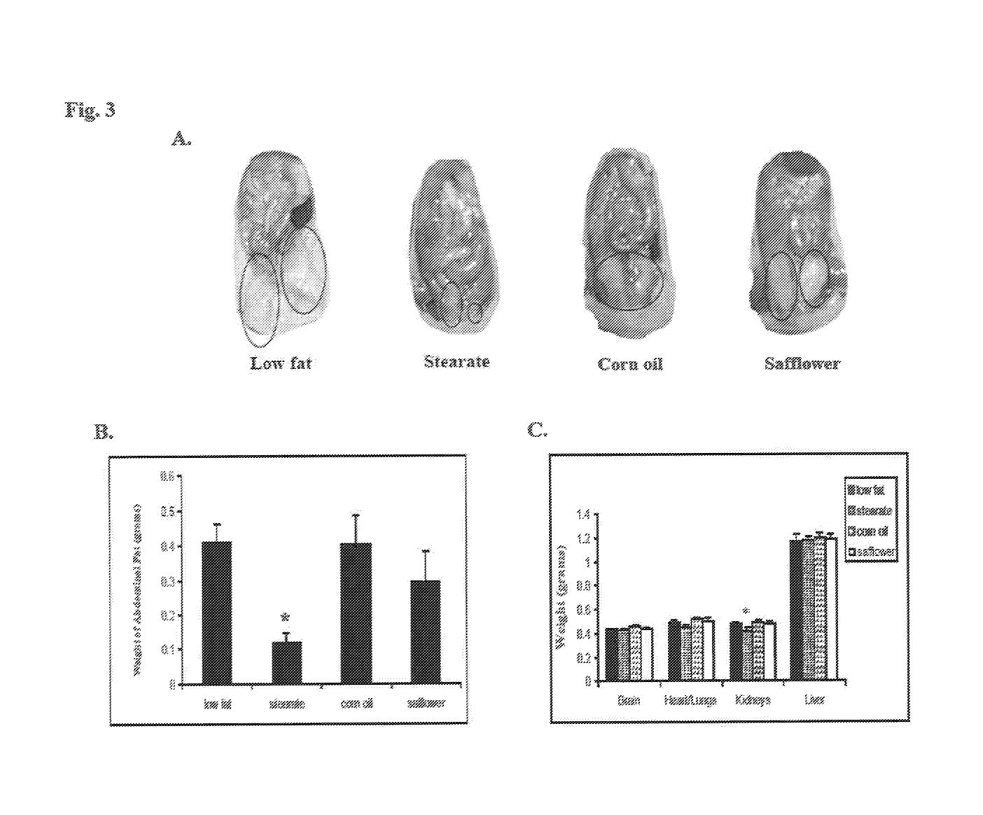

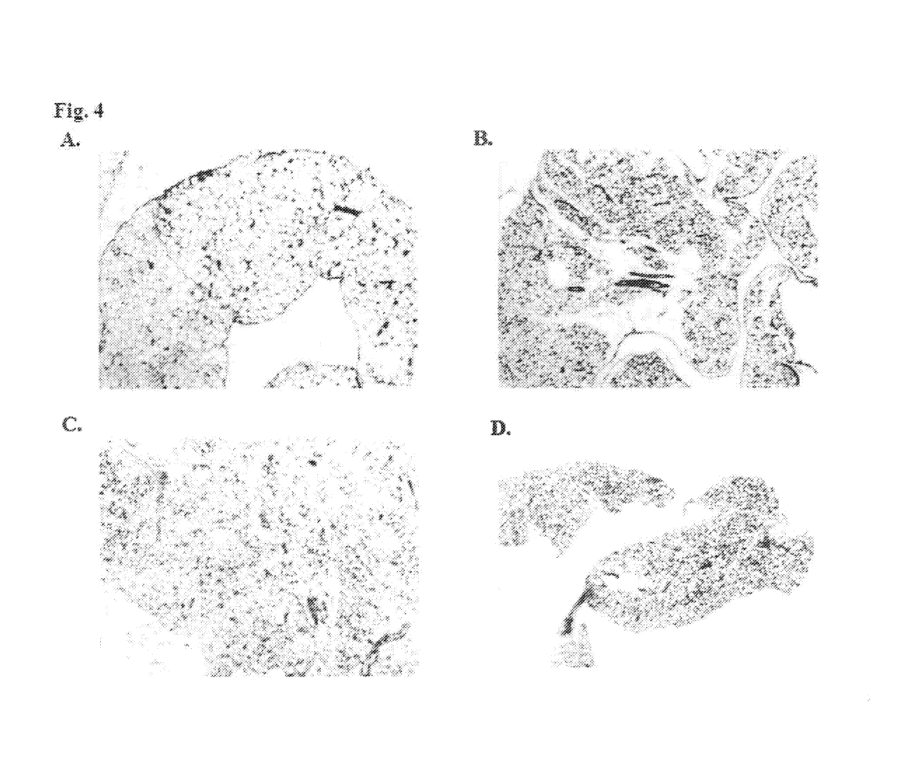

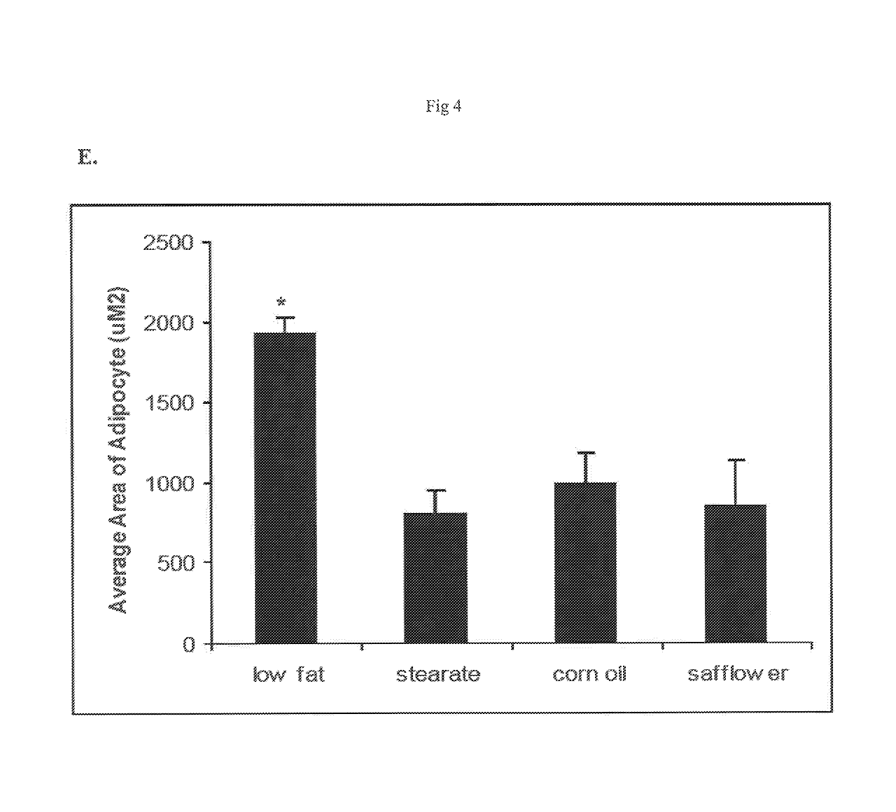

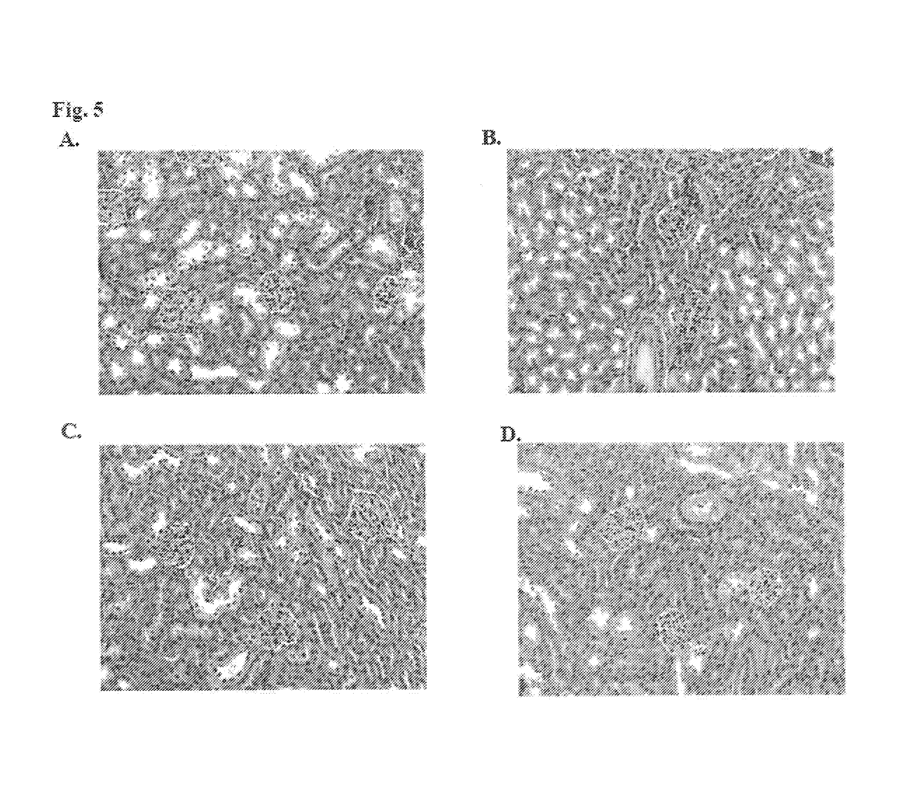

As is shown in FIGS. 3, A and B, abdominal fat was found to be decreased by a dramatic 70% in the stearate diet group when compared to the low fat diet group. Abdominal fat histological sections were prepared, stained with H&E and the average size of adipocytes was measured with histomorphometry. As shown in FIG. 4, mice on the low fat diet had significantly increased adipocyte size when compared to the stearate, corn oil and safflower groups.

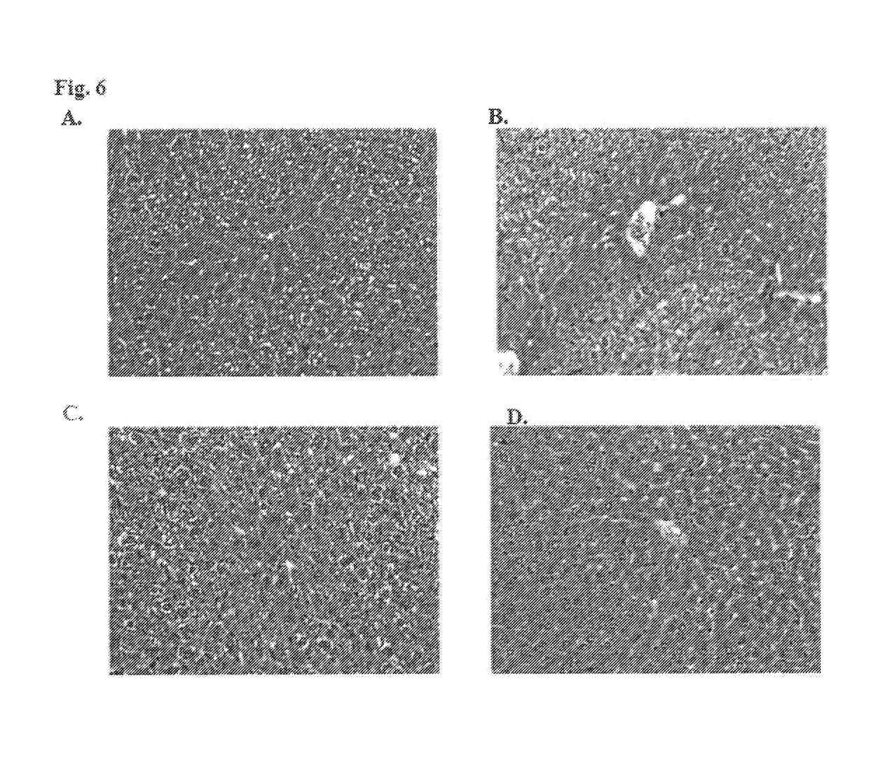

Although the weight of brain, heart/lungs and liver were similar among the different dietary groups, the weight of the kidneys was found to be modestly, but significantly, decreased in the stearate diet group (FIG. 3 C). Kidney and liver histological sections were prepared, stained with H&E and evaluated by two experienced pathologists. As shown in FIGS. 5 and 6, no meaningful pathological changes were found.

The Effect of Dietary Stearate on Serum Glucose, Insulin, and Inflammatory Cytokines

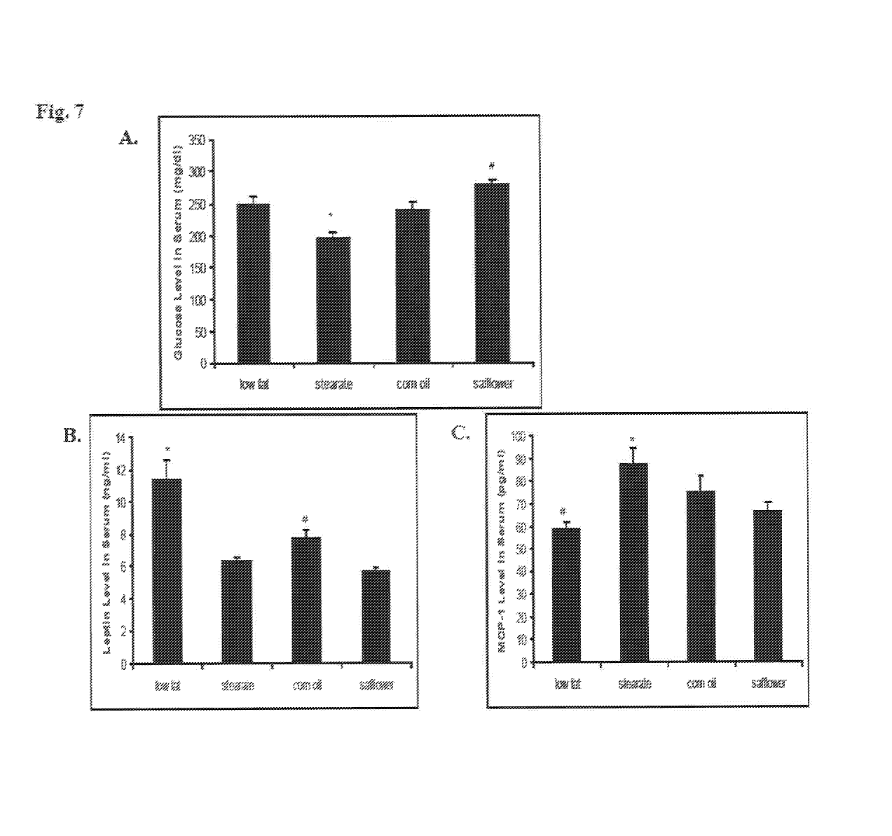

Possible changes in serum glucose, insulin and cytokine concentrations were then determined. At the end of the experiment, serum glucose, insulin, leptin, MCP-1, IL-6, and adiponectin were measured. Serum glucose and leptin were significantly decreased (FIGS. 7 A and B), while serum MCP-1 was significantly increased in the stearate diet group (FIG. 7 C). The serum level of insulin, IL-6 and adiponectin were the same among the different diet groups (p=0.46, p=0.46, p=0.074, respectively; later figures not shown).

The Effect of Stearate on the Differentiation of 3T3L1 Cells

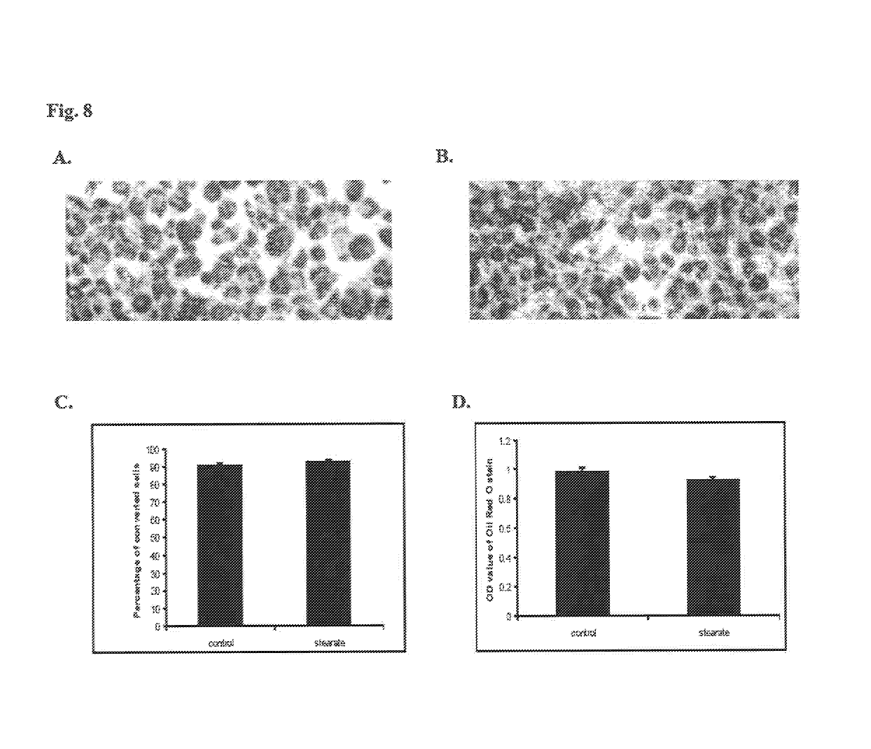

In order to investigate the possible mechanism of fat reduction caused by stearate, the direct effect of stearate on the differentiation of mouse 3T3L1 preadipocyte cells was examined. These cells can be induced to differentiate into adipocytes⁽⁴³⁾. 3T3L1 cells were treated with 50 μM stearate during the differentiation process and subsequently stained with oil red O to determine the fat accumulation. As shown in FIG. 8 A-D, adipocyte differentiation is not affected by stearate.

The Effect of Stearate on Cell Death of Adipocytes

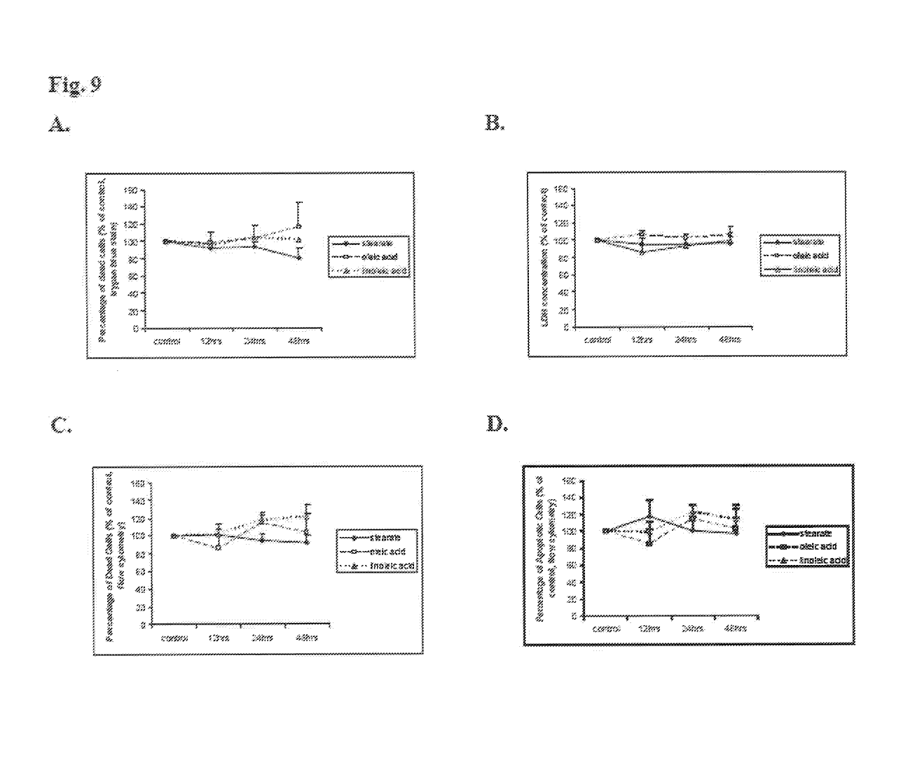

Induction of programmed cell death (apoptosis) by adipocytes through direct contact with stearate may be a driving force for body fat reduction. 3T3L1 cells were first converted into adipocytes and then treated with stearate, oleate or linoleate, and the percentage of apoptotic and necrotic cells was examined. Stearate, oleate and linoleate had no effect on the percentage of injured, apoptotic or dead cells (FIG. 9).

The Effect of Stearate on Cell Death of Preadipocytes

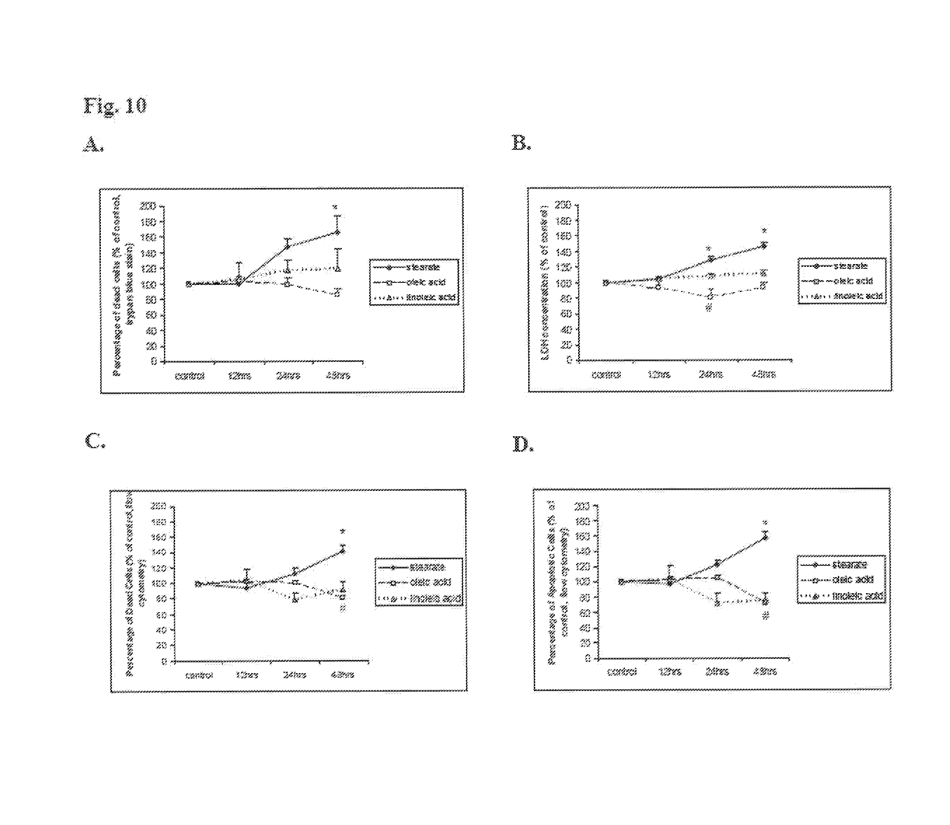

Undifferentiated 3T3L1 cells were used to determine whether stearate has a direct effect on preadipocytes. Stearate significantly increased the percentage of dead preadipocytes, while oleate significantly decreased the percentage of both apoptotic and necrotic cells (FIG. 10). Linoleate had no significant effects. FIG. 10 A shows that stearate increased preadipocyte cytotoxicity as measured by trypan blue exclusion; FIG. 10 B also shows stearate increased cell injury while oleate decreased cell injury as measured by lactate dehydrogenase in the media; FIG. 10 C shows similar results when flow cytometry is used to detect these cells; FIG. 10 D indicates that stearate increased apoptosis of preadipocytes while oleate decreased apoptosis as measured by flow cytometry.

In order to verify that the effect of dietary stearate on preadipocytes is at least in part an apoptotic effect, caspase-3 activity was measured after preadipocytes were treated with stearate. As shown in FIG. 10 E, caspase-3 activity increased significantly after 48 hours treatment, which is consistent with the flow cytometric results.

It has been shown for the first time that dietary stearate selectively reduces visceral fat compared to both a low fat control diet and a corn oil diet. In addition, dietary stearate causes apoptosis of preadipocytes. In contrast, stearate has no direct effect on fully differentiated adipocytes, and does not affect fat cell differentiation.

### Discussion

It has been demonstrated that dietary stearate selectively reduces abdominal fat. The significance of these studies comes from potential applications to breast cancer, cardiovascular disease (CVD) and diabetes. Approximately ⅔ of the US adult population is overweight and ⅓ is obese (1). Interestingly, increased visceral adipose tissue (VAT) or visceral obesity is even more prevalent than obesity ˜43% (2). Obesity is associated with CVD; however, this risk is mainly due to increased VAT (3, 4). Thus selectively reducing VAT may improve and/or prevent several diseases and pathologic conditions including CVD. Obese women, when stage and grade matched, are more likely to have a poor outcome and/or increased mortality (9-15), due in large part to breast cancer metastasis since most cancer patients die of metastasis (16). Visceral obesity as measured by computed tomography demonstrated that breast cancer patients had 45% more visceral fat/total fat (p<0.001) compared with control subjects that were matched for age, weight, and waist circumference (21). A similar small study was done for prostate cancer where controls were matched for BMI and age. They found that prostate cancer patients also had a significantly higher mean visceral fat/subcutaneous fat area, 50% more (p<0.001) (22).

Finally, while obesity is widely known to be associated with type 2 diabetes, visceral fat is more closely associated with this disease. Thus, it is possible that dietary stearate via reducing abdominal fat may be beneficial for CVD, breast cancer and type 2 diabetes.

It has been shown that dietary stearate does not increase cholesterol, unlike palmitate, nor does it increase low density lipoprotein or “bad” cholesterol (10). In addition dietary stearate does not adversely affect insulin action (11), is not thrombogenic (12, 13), does not affect blood pressure (14), is slowly metabolized and is preferentially incorporated into membrane phospholipids (15, 16) in human studies.

A recent paper indicated that high levels of dietary stearate promote adiposity and deteriorate hepatic insulin sensitivity in mice (6). This study utilized diets where ˜15% of the fatty acids were in the form of stearate and much higher percentages of palmitate and oleate were present. While the experiments were somewhat controlled for the presence of other fatty acids, the relatively large concentrations of other fatty acids compared to stearate raises the issue of them influencing stearate metabolism. For example, palmitate is known to cause insulin resistance and raise total and LDL cholesterol concentrations in the blood (18, 22). In our study we used a stearate diet in which 85% of the dietary fatty acids were stearate as compared to 15% stearate in the other study. The advantage of this approach is that it focuses on a stearate effect, while keeping other fatty acids at a minimum concentration necessary for normal growth and development. In previous studies in both mouse and rat models, dietary stearate animals did not vary significantly in weight compared to those on other diets including those on a low fat diet (1, and Carcinogenesis 32(8): 1251-1258 (2011)). Furthermore, we have found that dietary stearate actually lowers blood glucose concentrations and does not demonstrate a pathological affect on mouse liver or kidney. Dietary stearate has further been shown by us to reduce primary tumor burden, metastastatic tumor burden (1) and carcinogenesis (as explained below) in rodent mouse models of breast cancer and its metastasis. These data support dietary stearate as a candidate breast cancer inhibitor both for chemoprevention and therapy.

In summary it has been shown that dietary stearate selectively reduces visceral fat as well as lowers blood glucose and leptin concentrations. A specific effect of stearate causing apoptosis of preadipocytes but not mature adipocytes has been demonstrated. These studies provide a target to selectively reduce visceral fat and suggest that further investigations to determine the effects of dietary stearate on CVD, diabetes and the metabolic syndrome as well as certain cancers are indicated.

### REFERENCES

1. Penny M. Kris-Ethertona, Amy E. Griela, Tricia L. Psotaa, Sarah K. Gebauera, Jun Zhang, and Terry D (2005) Dietary Stearic Acid and Risk of Cardiovascular Disease: Intake, Sources, Digestion, and Absorption. Lipids (40):1193-1200.
2. Yu, S., Derr, J., Etherton, T. D., and Kris-Etherton, P. M. (1995) Plasma Cholesterol-Predictive Equations Demonstrate That Stearic Acid Is Neutral and Monounsaturated Fatty Acids Are Hypocholesterolemic, Am. J. Clin. Nutr. 61, 1129-1139.
3. Evans L M, Cowey S L, Siegal G P, Hardy R W. Stearate preferentially induces apoptosis in human breast cancer cells. Nutr Cancer. 2009; 61(5):746-53.
4. Evans L M, Toline E C, Desmond R, Siegal G P, Hashim A I, Hardy R W. Dietary stearate reduces human breast cancer metastasis burden in athymic nude mice. Clin Exp Metastasis. 2009; 26(5):415-24. Epub 2009 Mar. 8.
5. Wickramasinghe N S, Jo H, McDonald J M, Hardy R W. Stearate inhibition of breast cancer cell proliferation. A mechanism involving epidermal growth factor receptor and G-proteins. Am J Pathol. 1996 March; 148(3):987-95.
6. Dezhi Wang, Cecil R Stockard, Louie Harkins, Patricia Lott, Chura Salih, Kun Yuan, Donald Buchsbaum, Arig Hashim, Majd Zayzafoon, Robert Hardy, Omar Hameed, William Grizzle, and Gene P. Siegal. Immunohistochemistry for the evaluation of angiogenesis in tumor xenografts. Biotech Histochem. 2008 June; 83(3): 179-189.
7. Hardy R W, Gupta K B, McDonald J M, Williford J, Wells A. Epidermal growth factor (EGF) receptor carboxy-terminal domains are required for EGF-induced glucose transport in transgenic 3T3-L1 adipocytes. Endocrinology. 1995 February; 136(2):431-9.
8. Green H, Meuth M. An established pre-adipose cell line and its differentiation in culture. Cell 1974; 3: 127-133.
9. Sjoerd A A van den Bergl, Bruno Guigas, Silvia Bijland, Margriet Ouwens, Peter Voshol, Rune R Frantsl, Louis M Havekes, Johannes A Romijn, Ko Willems van Dijk. High levels of dietary stearate promote adiposity and deteriorate hepatic insulin sensitivity. Nutrition & Metabolism 2010, 7:24.
10. Kather H, Walter E, Simon B. Adipose tissue and obesity. Part 1: fat cell size and fat cell number. Fortschr Med. 1978 September; 96(34):1693-6.
11. Gurr M I, Kirtland J, Phillip M, Robinson M P. The consequences of early overnutrition for fat cell size and number: the pig as an experimental model for human obesity. Int J Obes. 1977; 1(2):151-70.

### Example 2

### Prevention of Carcinogenesis and Inhibition of Breast Cancer Tumor Burden by Dietary Stearate

This example demonstrates that stearate, at physiological concentrations, inhibits cell cycle progression in human breast cancer cells at both the G1 and G2 phases. Stearate also increases cell cycle inhibitor p21^{CIP1/WAF1} and p27^{KIP1} levels and concomitantly decreases cyclin-dependent kinase 2 (Cdk2) phosphorylation. The data also show that stearate induces Ras-guanosine triphosphate formation and causes increased phosphorylation of extracellular signal-regulated kinase (pERK). The MEK1 inhibitor, PD98059, reversed stearate-induced p21^{CIP1/WAF1} upregulation, but only partially restored stearate-induced dephosphorylation of Cdk2. The Ras/mitogen-activated protein kinase/ERK pathway has been linked to cell cycle regulation but generally in a positive way. Interestingly, stearate both inhibits Rho activation and expression in vitro. In addition, constitutively active RhoC reversed stearate-induced upregulation of p24^{KIP1}, providing further evidence of Rho involvement. To test the effect of stearate in vivo, the N-Nitroso-N-methylurea rat breast cancer carcinogen model was used. Dietary stearate reduces the incidence of carcinogen-induced mammary cancer and reduces tumor burden. Importantly, mammary tumor cells from rats on a stearate diet had reduced expression of RhoA and B as well as total Rho compared with a low-fat diet. Overall, these data indicate that stearate inhibits breast cancer cell proliferation by inhibiting key check points in the cell cycle as well as Rho expression in vitro and in vivo and inhibits tumor burden and carcinogen-induced mammary cancer in vivo.

Stearate (C18:0), a long-chain saturated fatty acid, has been reported to inhibit human breast cancer cell proliferation in vitro (1, 2) and in vivo (3). This effect contrasts increased cell proliferation observed in vitro with n-6 fatty acids such as linoleate and oleate (2, 4). The molecular basis for the inhibition of breast cancer cell proliferation by stearate is not known.

The epidermal growth factor receptor (EGFR) is frequently upregulated in human cancers including those thought to arise from the colon, head and neck, breast, pancreas, lung, kidney, ovary, brain and urinary bladder (5). Overexpression of EGFR in breast cancers is associated with a more aggressive clinical course suggesting that it has an important growth regulatory function (6, 7). The stimulation of EGFR with EGF regulates the proliferation, motility and differentiation of cells through activation of several intracellular signal transduction cascades, including the Ras/Erk and Rho/cyclin kinase inhibitor signaling pathways (8). The Ras superfamily of guanosine triphosphatases (GTPases) is a master regulator of many aspects of cell behavior. There are at least 60 small molecular weight, monomeric GTPases in mammalian cells and they have been generally divided into five groups Ras, Rho, RAb, Arf and Ran. They function as switches in signal transduction pathways that regulate such important functions as cell growth, differentiation and survival (9). In cancers with wild-type Ras, such as seen in most breast cancers, growth factor overexpression frequently leads to activation of the Ras/extracellular signal-regulated kinase (ERK) signaling pathway suggesting that Ras makes an important contribution to the development of these human cancers (10). In breast cancer, there is upregulated signaling through multiple pathways, and molecules implicated include growth factor receptors and other tyrosine kinases, Ras regulators commonly found to be overexpressed, the Ras protein itself; as well as downstream effectors (10). Members of both the Ras and Rho subfamilies are known to affect cell proliferation. Over the last decade, it has been generally accepted that Ras and Rho signaling pathways cross talk in such a way as to favor transformation and cell proliferation (11, 12). The present studies support these data and further show that stearate induces breast cancer cell cycle inhibition largely in G1 as well as inhibiting carcinogen-induced mammary cancer and Rho both in vitro and in vivo.

### Materials and Methods

Antibodies and Reagents

Antibodies used and their sources were: Ras (clone RAS10 Mouse IgG2a) from Oncogen (Boston, Mass.), p27^{KIP1} (clone F-8 mouse IgG1), cyclin-dependent kinase 2 (Cdk2, rabbit polyclonal IgG) from Santa Cruz Biotechnology (Santa Cruz, Calif.), p21^{CIP1/WAF1} (clone SX118 mouse IgG1) from BD Biosciences PharMingen (San Diego, Calif.), phosphorylated Cdk2 [pCd1c2(Thr160)] and phosphorylated p44/42 ERK [pERK1(Thr202)/pERK2(Tyr204)] from Cell Signaling (Beverly, Mass.). 2′-amino-3′-methoxyflavone (PD98059) and RNase inhibitor were purchased from Promega Corporation (Madison, Mich.). Stearic acid (stearate), diatomaceous earth, propidium iodide, RNase and protease inhibitor cocktail were obtained from Sigma-Aldrich Chemical Co. (St Louis, Mo.). Antirabbit or antimouse antibodies labeled with horseradish peroxidase and enhanced chemiluminescence reagents were from Amersham, Pharmacia Biotech (Piscataway, N.J.). All other chemicals were of reagent grade.

Cell Culture

Hs578T human breast cancer cells (ATCC, HTB-126) were maintained according to the manufacturer's recommendations, in Dulbecco's modified Eagle's medium containing 10% fetal bovine serum, 10 gg/ml insulin and penicillin/streptomycin.

Treatment of the Cells

The concentration of stearate used to treat the Hs578T cells was 50 μM. When EGF was used, the concentration was 1 nM EGF. Before the treatment with stearate or EGF, cells were first starved for 24-48 hours. Stearate was loaded onto fatty acid-free bovine serum albumin (BSA) according to the method reported by Spector et al. (13); briefly, stearate (0.5 g) was dissolved in chloroform (100 ml) and mixed well with 10 g diatomaceous earth in a 1 liter flask. The mixture was stirred and dried under nitrogen until powder. BSA is a physiological carrier of fatty acids and was used to avoid the introduction of organic solvents to solutions coming into contact with cells. Fatty acid-free BSA (1 g) was dissolved in 100 ml with Dulbecco's modified Eagle's medium without phenol red and mixed with 3 g of the stearate/diatomaceous earth mixture with stirring for 45 min. The stearate/BSA solution was filtered through a 0.45 μm filter, and adjusted to pH 7.4. The concentration of stearate in the solution was detected with the NEFA C Kit from Wako Chemicals GmbH (Neuss, Germany). All experimental data on Hs578T cells were controlled using fatty acid-free BSA control solutions that were put through the same preparatory procedure described for the stearate/BSA solution except for the fact that no fatty acid was added.

Transfection of Constitutively Active Mutant RhoA, RhoB and RhoC

Constitutively active mutant 3×HA epitope-tagged (N-terminus) RhoA, RhoB and RhoC proteins were purchased from the University of Missouri-Rolla, cDNA Resource Center (Rolla, Mo.). Hs578T cells (105) were cultured in a 35 mm culture dish with complete medium until they were 50-80% confluent. No antibiotics were provided during the 24 h before transfection. The transfection was done according to the manufacturer's instructions for use of the FuGENE 6 so Transfection Reagent (Roche, Indianapolis Ind.).

Flow Cytometry for Cell Cycle Analysis

To analyze cellular DNA content, confluent Hs578t cells were harvested, fixed in ice-cold 70% ethanol for 30 min and then resuspended in citrate buffer (4 mM sodium citrate) containing 50 μg/ml of propidium iodide and 100 μg/ml of RNase. After a 20 min incubation at room temperature, cells were run on FACScan flow cytometry. Data were analyzed using the ModFit LT workshop program (BD Immunocytometry System, San Jose, Calif.).

Ras and Rho Activation Assay

Ras and Rho activation assay kits were purchased from Millipore (Billerica, Mass.). The activation assay followed the protocol of the manufacturer. Briefly, after cells were treated and the lysates prepared, 1 mg protein (supernatant) was incubated with Rhotekin Rho-binding domain (25 μg)-agarose and then Raf-1/Ras binding domain (10 μg)-agarose beads at 4° C. for 45 min. The beads were washed three times with lysis buffer B. Bound Ras-GTP and Rho-GTP proteins were detected by immunoblot using Ras and Rho antibodies.

Immunoblot

Cells were treated as described above, and lysed with lysis buffer. The supernatants of the lysates or the immunoprecipitates were loaded with Laemmli sample buffer on 10% sodium dodecyl sulfate-polyacrylamide gel electrophoresis gels after boiling at 100° C. for 5 min. Proteins were then transferred to a polyvinylidene difluoride membrane. The membranes were blocked overnight at 4° C. with blocking buffer containing 5% non-fat dried milk powder in Tris-buffered saline-T (25 mM Tris, 140 mM NaCl, 2.7 mM KCl, 0.05% Tween-20, pH 8.0), incubated with primary antibody in blocking buffer at room temperature for 1 hour and incubated with antirabbit or antimouse antibodies labeled with horseradish peroxidase (1:5000) in blocking buffer under the same conditions, and then washed three times for 10 min in Tris-buffered saline-T. The polyvinylidene difluoride membranes were washed and developed using enhanced chemiluminescence reagents.

Quantitative Real-Time Reverse-Transcription Polymerase Chain Reaction for p21^{CIP1/WAF1} and p27^{KIP1}

Total RNA was extracted and purified with TRIZOL Reagent (GIBCO Invitrogen, Carlsbad, Calif.). The first-strand complementary DNA (cDNA) synthesis was achieved using a commercially available kit (New England BioLabs, Beverly, Mass.) according to the protocol from the manufacturer. Briefly, 1 μg of total RNA was reverse-transcribed using M-MuLV reverse transcriptase (25 U) and dT23VN primer (5 μM) in a final volume of 25 μl.

Quantitative real-time reverse-transcription polymerase chain reaction (RT-PCR) cDNA samples were diluted to appropriate concentrations and used for a real-time RT-PCR assay in a volume of 25 μl, containing 2 μl DNA template, 12.5 μl SYBR® Green PCR Master Mix (Applied Biosystems, Foster City, Calif.) and 0.4 μM each specific primer. The gene expression of p21^{CIP1/WAF1} and p27^{KIP1} were determined by real-time quantitative RT-PCR and the house-keeping gene 18S ribosomal RNA (rRNA) was used as an internal control to normalize the variable RNA loading in each sample. Sequences of primer sets were as follows: human p21^{CIP1/WAF1} (sense 5′-GGC GGG CTG CAT CCA-3′; antisense 5′-AGT GGT GTC TCG GTG ACA AAG TC-3′), human p27KIP1 (sense 5′-CGG TGG ACC ACG AAG AGT TAA-3′; antisense 5′-GGC TCG CCT CTT CCA TGT C-3′) and 18S rRNA (sense 5′-CGC CGC TAG AGG TGA AAT TCT-3′; antisense 5′-CGA ACC TCC GAC TTT CGT TCT-3′). All pairs of primers were designed by the Primer Express program (Applied Biosystems) based on sequence information from the GenBank database. After annealing, at 50° C., for 2 min and an initial denaturation at 95° C. for 10 min, 40 repetitive cycles were carried out with denaturing, at 95° C., for 15 s, and annealing, at 60° C., for 60 min, using a GeneAmp® 5700 Sequence Detection System (Applied Biosystems). The comparative cycle threshold (CT) method was used to analyze the data generated from relative values of the amount of target cDNA. CT represents the number of cycles for the amplification of target cDNA to reach a fixed threshold and correlates with the amount of the starting material present. The fluorescence intensity corresponding to the C_{T} was used to quantitate the target cDNA in the mixture of samples with 103-fold dilutions, employing the standard curve for each target gene. Sequence Detector Software (Applied Biosystems) was used to extract the data of the quantitative real-time RT-PCR. The calculated result represents the relative expression levels of target genes compared with its expression in the control group after the value of target genes was normalized to 18S rRNA expression levels.

Animal and Diets

All animal protocols were approved by the University of Alabama at Birmingham, Institutional Animal Care and Use Committee. Ninety-five female Sprague Dawley rats (Harlan) obtained at 21 days of age were used for the in vivo experiments. The animals were housed two to three rats per cage, and had free access to food and drinking water. The animals were randomly assigned to one of three of the following diets made by Harlan-Teklad: (i) low-fat diet (8.5% fat); (ii) stearate diet (17% fat by weight); and (iii) safflower oil diet (17% fat by weight). The weight and food intake were monitored three times a week. At 50 days of age, the animals were injected with 50 mg/kg N-Nitroso-N-methylurea (NMU). The size of NMU-induced tumors were measured weekly after 42 days post-injection. The experiment was ended 100 days post-injection. Tumor samples were preserved in a 10% paraformaldehyde solution.

Tumor Microdissection and RT-PCR for Rho

Microdissection of specimens for PCR analysis was done at the University of Alabama at Birmingham Laser Microdissection Laboratory. Briefly frozen sections were fixed in 70% ethanol and stained with hematoxylin and eosin. Tumor cells were microdissected from the sections using a laser capture microdissection system with an infrared diode laser (PixCell II System, Arcturus Engineering, Mountain View, Calif.). Total RNA was extracted and purified with RNaqueous Micro Kit (Applied Biosystems/Ambion, Austin, Tex.). The first-strand cDNA synthesis was achieved using the iScript cDNA Synthesis Kit (Bio-Rad, Hercules, Calif.).

In total, 30-40% confluent Hs578T cells were treated with stearate (50 μM) for 48 hours after starvation for 24 hours. Total RNA was extracted and reverse-transcribed as described in RT-PCR for p21^{CIP1/WAF1} and p27^{K1P1}.

The PCR assay was performed in a volume of 50 μl, containing 4μl DNA template, 45 μl Platinum PCR Supermix (Invitrogen) and 0.2 μM each specific primer. PCR specificity and efficiency were improved by using hot start PCR with 3 min pre-denaturation, at 95° C., and 30 cycles of denaturation (95° C., 30 s), annealing (52° C., 30 s) and 1 min extension (72° C.). The PCR products (20 INS>μl) were analyzed by means of 1% agarose in tris-acetate ethylenediaminetetraacetic acid gel electrophoresis and visualized by ethidium bromide staining under ultraviolet; digital images were analyzed by means of a FUJI Medical System (FUJIFILM) and the bands quantified by Quantity Software (FUJIFILM). Tris-buffered saline was used to normalize the variable RNA loading in each sample. The calculated result represents the relative expression levels of target genes compared with its expression in the control group after the value of target genes was normalized to GAPDH expression levels.

### Results

### Stearate Inhibits Cell Cycle Transition of the G₀/G₁ to S and G₂/M to G₀/G₁ Phases

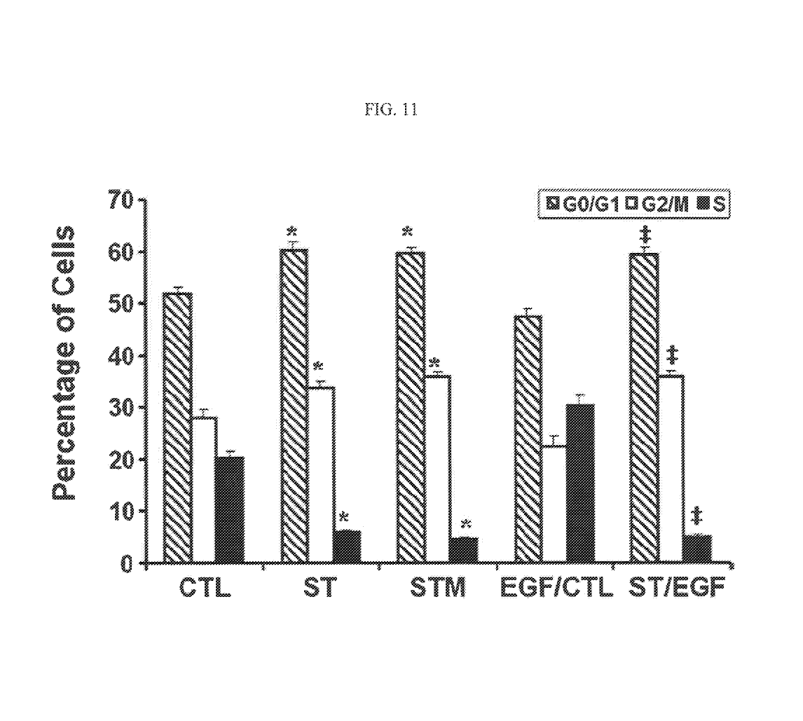

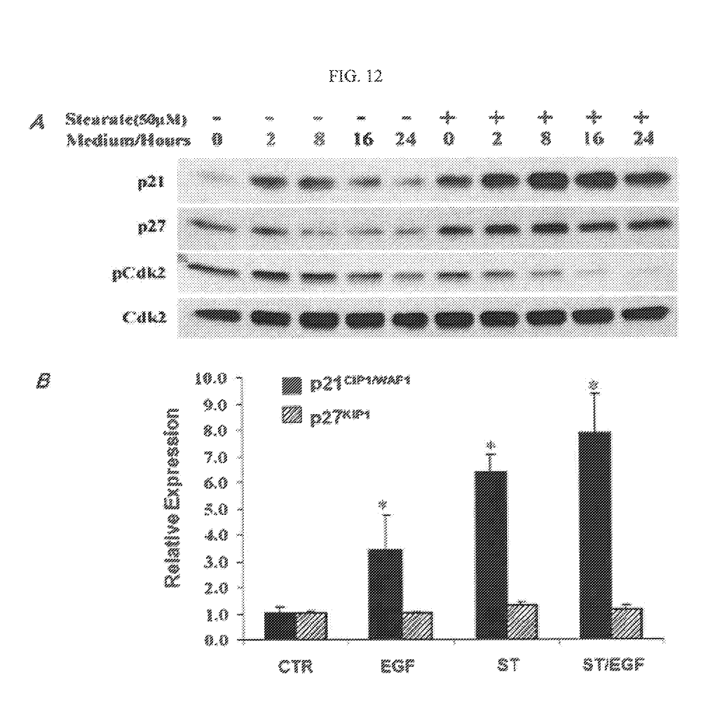

As shown in FIG. 11, stearate treatment increased the number of cells in G₀/G₁ and G₂/M and reduced cells in the S phase. These effects persisted even in the presence of complete media and addition of exogenous EGF for up to 16 h. These data suggest a cell cycle-mediated inhibition of proliferation of Hs578T human breast cancer cells.

Stearate increases p21^{CIP1/WAF1} and p27^{KIP1} and Inhibits Phosphorylation of Cdk2

All major transitions of the eukaryotic cell cycle (G₀/G₁, G₁/S and G₂/M) are controlled by the activity of Cdks (14). The activity of Cdks is carefully regulated by the formation of heterodimeric complexes of Cdks with their positive regulatory subunit (cyclins) and negative regulators, including p21^{CIP1/WAF1} and p27^{KIP1} (14, 15). Both p21^{CIP1/WAF1} and p27^{KIP1} have been implicated in G₁ arrest and high levels of p21^{CIP1/WAF1} can also lead to G₂ arrest. It was hypothesized that stearate may increase the expression of p21^{CIP1/WAF1} and/or p27^{KIP1}.

Stearate increases the protein level of p21^{CIP1/WAF1} and p27^{KIP1} (FIG. 12 A). Consistent with these data, pCdk2 was decreased with stearate treatment (FIG. 12 A). These results indicate that decreased activation of Cdk2 in response to stearate, in combination with increased p21^{CIP1/WAF1} and p27^{KIP1}, probably halt cell cycle progression from G₀/G₁ to the S phase and also G₂/M progression.

It was then determined whether stearate induced a transcriptional response in p21^{CIP1/WAF1} and/or p27^{KIP1}. FIG. 12 B shows that the increased protein level of p21^{CIP1/WAF1} was due to increased gene expression whereas increased protein level of p27^{KIP1} was not, suggesting that p27^{KIP1} degradation might be inhibited by stearate.

Stearate Upregulates p21^{CIP1/WAF1} Via Ras Activation and ERK Phosphorylation

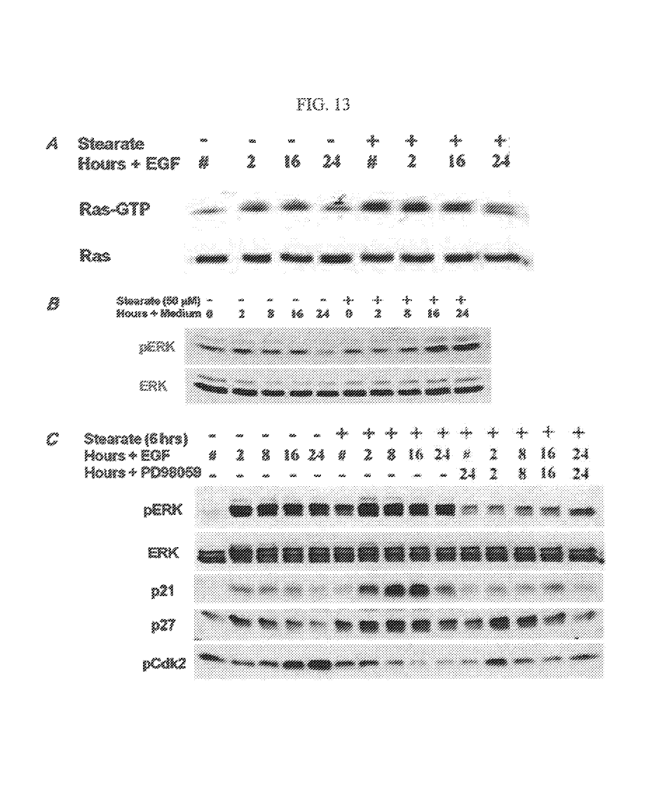

GTP loading of Ras plays a crucial role in cell cycle progression and the downstream activation of ERK (16). Whether stearate influences Ras and ERK activities was investigated. Stearate increases the binding of GTP to Ras (FIG. 13 A) with or without EGF. It was also found that phosphorylation of ERK increased between 8 and 16 h post-stearate treatment and that this was sustained up to 24 h after stearate treatment (FIG. 13B).

To investigate the role of the ERK signaling pathway in the stearate-induced cell cycle arrest, we examined the effects of a specific inhibitor of MEK1, PD98059, on regulation of protein levels and phosphorylation of cell cycle-related molecules. As shown in FIG. 13 C, PD98059 blocked ERK phosphorylation induced by stearate and EGF, indicating that ERK activation by stearate is MEK1-dependent and, therefore, likely linked to Ras activation. Addition of PD98059 to cells following stearate and EGF treatment reverses the upregulation of p21^{CIP1/WAF1}, indicating that the p21^{CIP1/WAF1} response to stearate is dependent on ERK signaling. However, PD98059 did not reverse the increases in p27^{KIP1} and only partially reversed the decrease in pCdk2 in stearate-treated cells, indicating that stearate-induced changes in p27^{KIP1} are independent of ERK signaling. This raised the possibility that other signaling pathways linked with p27^{KIP1} and pCdk2 may coexist.

Stearate Upregulates p27^{KIP1} Via Rho Inhibition

Expression of Rho family molecules has been reported in breast, lung, pancreas, colon carcinomas and in testicular germ cell tumors (17-21). The consequences of activated Ras-ERK signaling depend on Rho activity (12, 22). Thus Rho activity was examined.

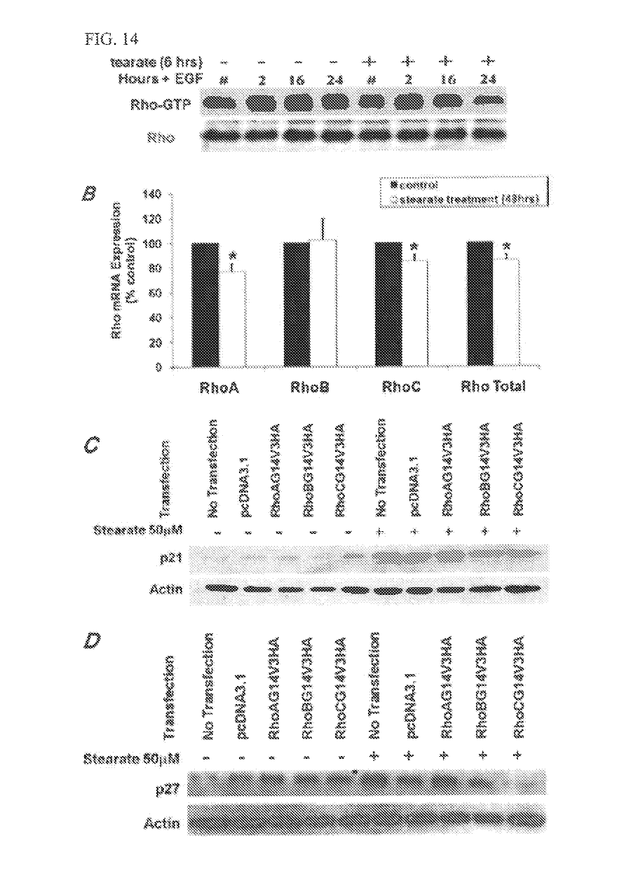

In FIG. 14 A, EGF increased Rho-GTP formation at 2 and 16 h, which returned to approximately basal levels at 24 h. Stearate decreased Rho-GTP at all time points tested, especially at 16 and 24 h post-EGF stimulation, compared with controls.

Rho messenger RNA (mRNA) expression was also examined. It was found that neither EGF nor stearate affected the mRNA expression over 24 h. However, when cells were treated with stearate for 48 h, the mRNA expression of RhoA, RhoC and total Rho significantly decreased, whereas RhoB remained unchanged (FIG. 14 B). These data indicate that while stearate activates Ras, it simultaneously inhibits Rho activation and on longer exposure, Rho mRNA expression, indicating an inhibition of Ras-Rho cross talk. The decreased Rho mRNA expression may contribute to a further reduction in Rho activation after 48 h.

In order to identify the role of Rho activity in the regulation of the cell cycle regulatory protein p21^{CIP1/WAF1} and p27^{KIP1}, Hs578T cells were transfected with constitutively active RhoA, RhoB and RhoC, and the cells were treated with/without stearate for 6 h. An immunoblot of p21^{CIP1/WAF1} and p27^{KIP1} showed that constitutively active RhoC reverses the effect of stearate on p27^{KIP1}, but not that on p21^{CIP1/WAF1} (FIGS. 14 C and D). These data indicate that the upregulation of p27^{KIP1} in response to stearate is dependent on RhoC inhibition.

Dietary Stearate Inhibits NMU-Induced Mammary Tumors and Rho mRNA Expression

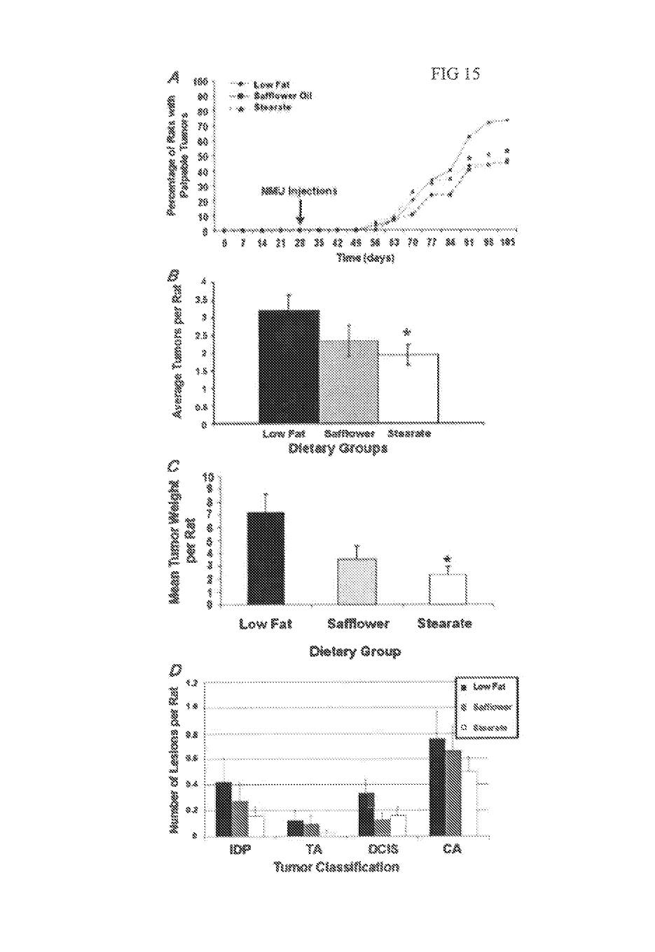

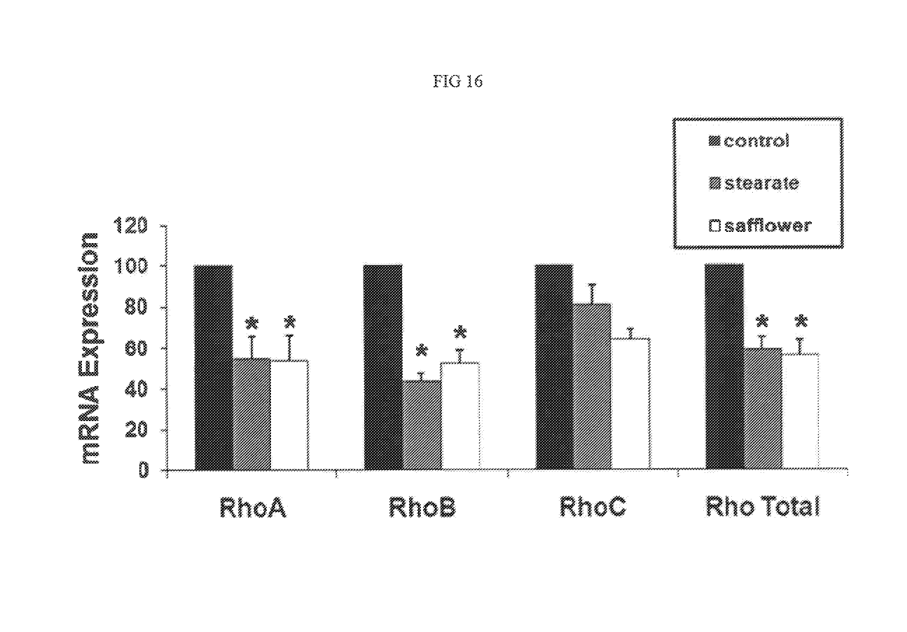

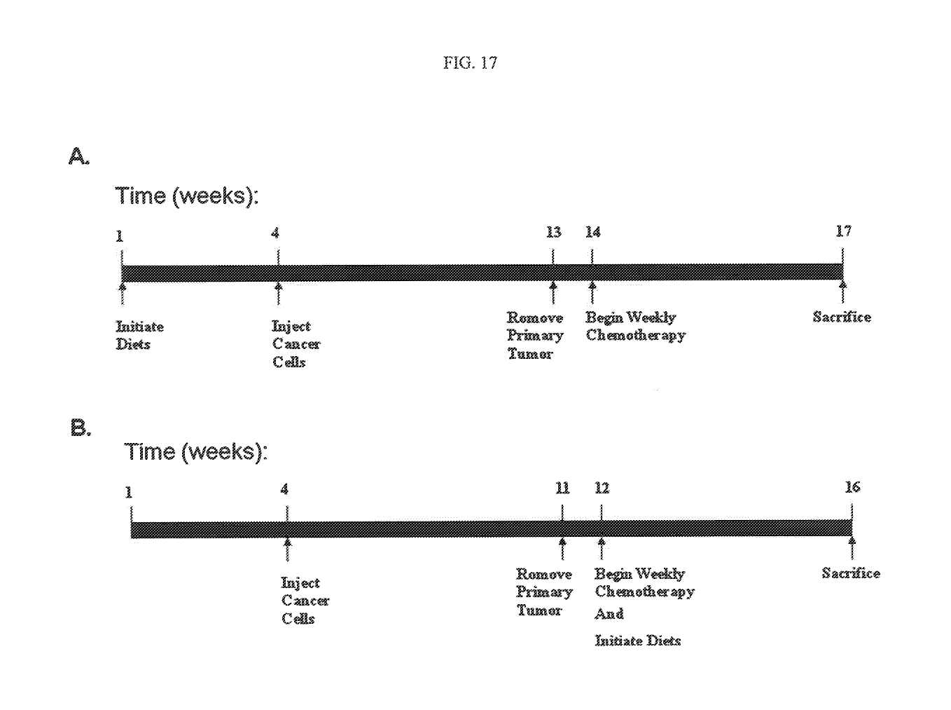

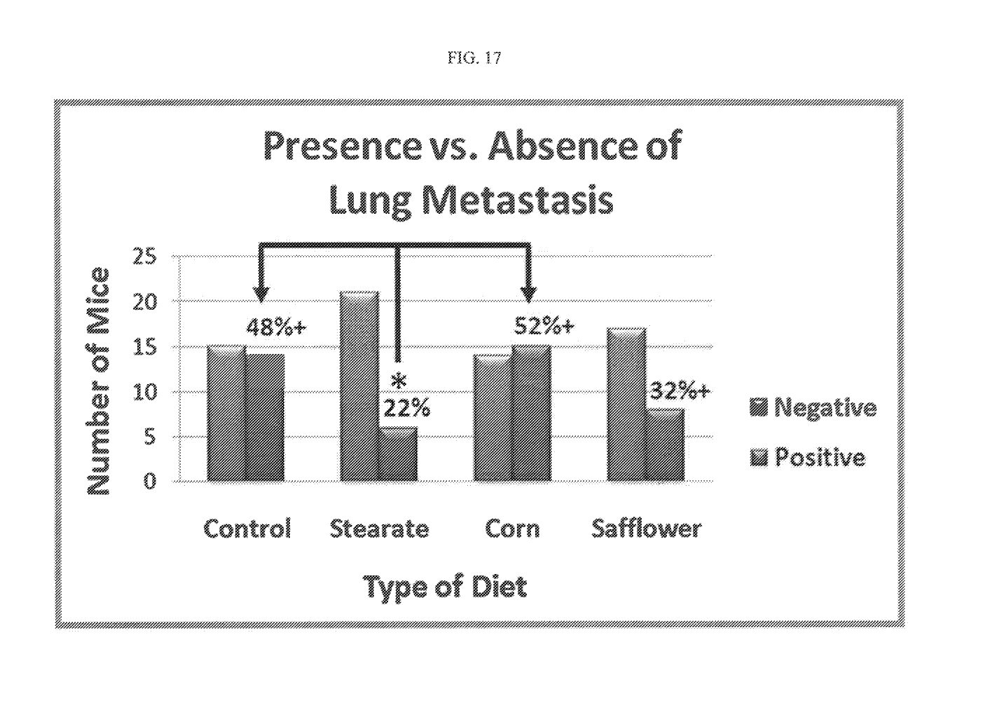

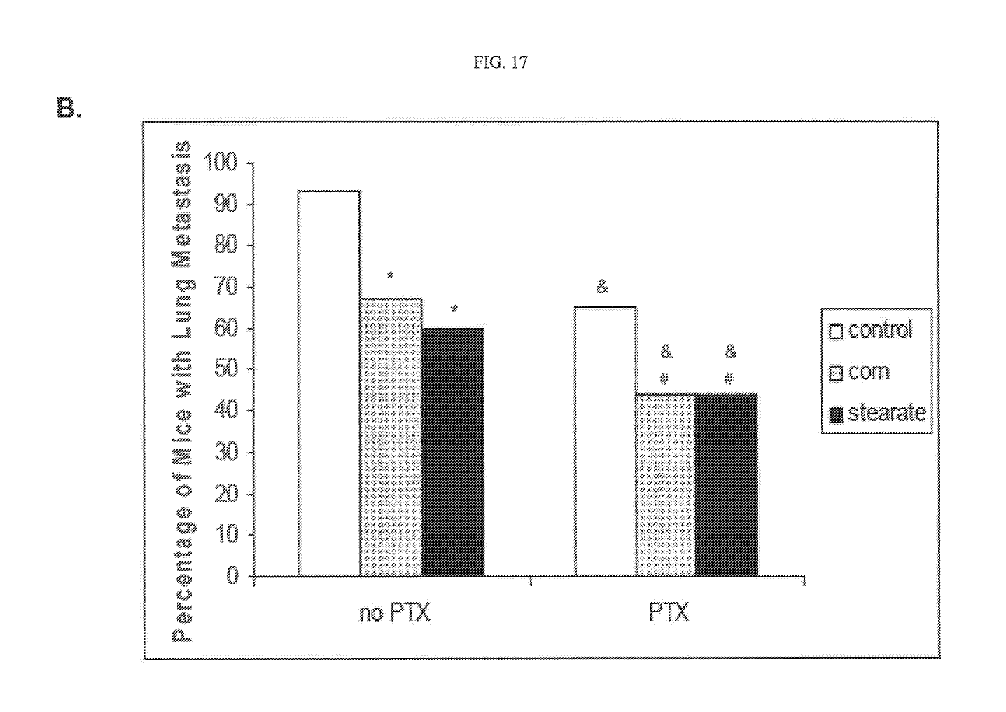

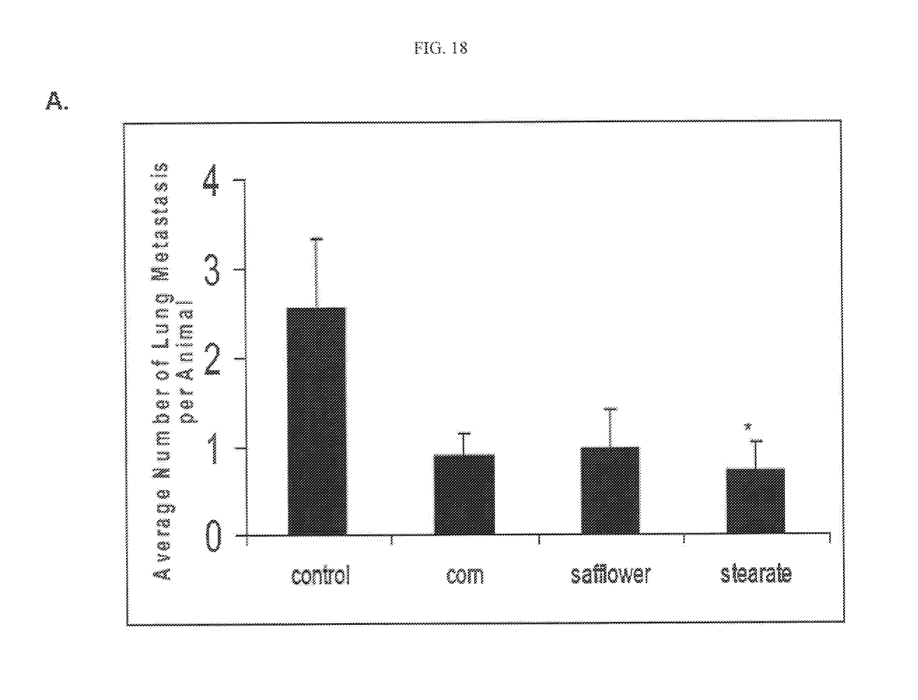

In order to determine whether stearate inhibits Ras-Rho cross talk in vivo and its effects on breast cancer carcinogenesis in terms of cell transformation, we used the NMU rat mammary cancer carcinogen model. It was found that dietary stearate significantly reduced the incidence of mice with tumors that developed over 15 weeks compared with the low fat diet, as did the safflower oil diet (FIG. 15 A). However, the average number of tumors per rat was only significantly decreased in the stearate diet compared with the low-fat diet (FIG. 15 B). In addition, tumor burden as defined by average tumor weight per rat was significantly decreased in the stearate diet compared with the low-fat diet (P<0.001), with the safflower oil diet not reaching significance compared with the low fat (P 5 0.057, FIG. 15 C). When the tumors were classified into four categories by a diagnostic pathologist using the method of Chan et al. (23), intraductal proliferations, tubular adenoma, ductal carcinoma in situ and adenocarcinoma, we found that compared with the low-fat group, the average number of tumors per animal in the stearate group decreased in all the categories (FIG. 15 D); however, there were no significant differences found between dietary groups in this analysis. Importantly, the mRNA expression of RhoA, RhoB and total Rho of microdissected tumor cells were significantly decreased in both stearate and safflower groups (FIG. 16) confirming stearate inhibition of Rho expression in vitro (FIG. 14 B).

In summary, stearate inhibits breast cancer cell cycle in G₁ and to a lesser extent G₂ while at the same time increasing cell cycle inhibitors p21^{CIP1/WAF1} and p24^{KIP1} and decreasing phosphorylation of Cdk2. Stearate also decreased Rho activation and expression in vitro and Rho expression in vivo while decreasing NMU-induced mammary cancer incidence and tumor burden.

### Discussion

Long-chain saturated fatty acids are a major component of dietary fat. The present studies indicate that stearate arrested cell cycle progression from G₁ to S and to a lesser extent from G₂ to M. These results differ from several studies demonstrating stearate-induced cell death/apoptosis (24-26). This may be due to differences in the concentration of stearate used, time of exposure and cell type. Typically, the minimum concentration of stearate used in previous studies demonstrating was 100 μM, which is double the concentration used in these experiments and other manuscripts use even higher, non-physiologic, concentrated preparations. In one study, treating human ovarian granulosa cells with 50 μM stearate (or palmitate) for 3 days demonstrated no decrease in cell viability (24). Recent studies indicate both a time and concentration dependence of stearate to induce apoptosis in human breast cancer cells and that this effect is specific for breast cancer cells compared with non-cancer breast cells (27). The stearate concentration (50 μM) used in our present study was generally maintained for 6 h and represents a high normal physiological exposure with respect to concentration and time (28, 29). Thus, the experiments herein indicate an early stage of stearate exposure that precedes apoptosis.

Cell cycle entry and progression rely on the precisely controlled expression and activation of cell cycle-related enzymes, termed Cdks, cyclins and cyclin-dependent kinase inhibitors. The activity of Cdks is controlled by cyclin-binding interactions, regulated phosphorylation and association with cyclin kinase inhibitors (14). p21^{CIP1/WAF1} is a broad spectrum cell cycle inhibitor involved in G₁ to S and G₂ to M phase transitions (30) and increased p21^{CIP1/WAF1} would be expected to inhibit both cdc2 and cyclin E/Cdk2 complexes. p27^{KIP1} is also known to inhibit G1 progression via Cdk2 inhibition (15, 31, 32). Mitogens stimulate elimination of p27^{KIP1} by decreased translation and increased ubiquitin-directed degradation (33). EGF increased p27^{KIP1} degradation in Hs578T human breast cancer cells; however, stearate prevented EGF-induced p27^{KIP1} degradation from reaching control cell levels. It has been proposed that inhibition of p27^{KIP1} degradation results in elevated levels of p27^{KIP1} and inhibition of G1 progression (12, 22, 32). Thus, upregulation of both p21^{CIP1/WAF1} and p27^{KIP1} as observed in these experiments are linked with cell cycle arrest caused by stearate.

Cell transformation by oncogenic Ras has been shown to require the function of Rho. Rho GTPases such as RhoA, Rac1 and Cdc42 have been shown to be required for Ras-induced cell transformation (34-36). Subsequent studies indicate that Ras mobilizes not only the Raf-mitogen-activated protein kinase-ERK-mediated kinase signaling cascade but also the PI-3-kinase and RalGDS pathways for complete cell transformation (37). Exactly how Rho functions in the PI-3-kinase and RalGDS signaling pathways is not clear; however, it has been proposed that Rho signaling involves these two pathways in Ras transformation (38).

It is known that transformed cells have elevated levels of activated Rho that inhibit the expression of p21^{CIP1/WAF1} and induce cyclin D1 thereby promoting cell proliferation (22, 39). There is evidence indicating that palmitoylation and possibly acylation by stearate can increase Ras activity by promoting Ras association with the plasma membrane (48, 49). However, the mechanism whereby stearate inhibits Rho activity and expression is not yet known. One possibility of how Rho activity is inhibited by stearate is via inhibition of the translocation of p190 Rho-GAP to detergent insoluble membranes in response to Ras (40). The data in FIGS. 14 C and D are consistent with this hypothesis. Constitutively active Rho's were used that are not affected by p190 Rho-GAP and showed that constitutively activated Rho B and C were both able to at least partially reverse the effects of stearate on p21^{CIP1/WAF1} and p27^{KIP1} protein concentration.

In these experiments, although stearate increased Ras activity, it decreased Rho activation and mRNA expression. This may be the key as to how stearate inhibits cancer cell cycle progression. RhoA is known to stimulate p27^{KIP1} degradation by inducing cyclin E/Cdk2 activity (32, 33). Thus, blocking Rho activity and mRNA expression would be expected to lead to a decrease in both cyclin E/Cdk2 activity and p27^{KIP1} degradation which is exactly what happened with stearate treatment. Although in vitro data on Hs578T cells demonstrated that both RhoA and RhoC mRNA expression are inhibited by stearate, only constitutively active RhoC and to a lesser extent B inhibit the effect of stearate on cell cycle proteins p27^{KIP1} and p21^{CIP1/WAF1}. Interestingly, it was reported that upregulation of RhoC plays an important role in inflammatory breast cancer (41), as well as in other malignant neoplasms including those thought to arise in the urinary bladder, ovary, pancreas and skin (16, 42-44). Although a decrease in RhoC in the NMU model of mammary cancer was not seen, it is possible that RhoC does not play a major role in the development of this particular cancer. Consistent with this hypothesis is the fact that RhoC seems to be involved in aggressive forms of breast cancer and the NMU model develops a type of cancer that slowly progresses and did not demonstrate metastasis in our hands. It further indicates that RhoA and B may play important roles both in carcinogen-induced mammary cell transformation and cell proliferation. Although Ras mutations are important in 30% of cancers (9), the incidence of Ras point mutations in primary breast cancers is rare (<5%) (10). Nevertheless in breast cancer there is upregulated Ras signaling through growth factor receptors and other tyrosine kinases or Ras regulators commonly overexpressed, the Ras protein itself or downstream effectors (10). In the NMU rat model 80-90% of tumors are Ras dependent making it an ideal model to confirm the in vitro data that stearate inhibits Rho and thus cell transformation and tumor growth in vivo (45, 46). Although this study focuses tightly on dietary stearate, the cell cycle, Rho and Ras, a consideration of other mechanistic studies concerning fatty acids and cancer provides better perspective of how the present studies may fit in this field. Dietary fat has been suggested to promote the development of cancer via altering cellular membrane structure (47). Membrane lipid structure can affect membrane-bound proteins thereby influencing intracellular signaling. Importantly, stearate is preferentially incorporated into phospholipids such as phosphatidylinositol (48). One of the other signaling systems that may be affected by dietary stearate is the protein kinase C (PKC) pathway. PKC is activated by phospholipases including phosphoinositide phospholipase C which when activated produces diacylglycerol, a co-activator of classical PKCs and 1,4,5-trisphosphate that stimulates the release of intracellular calcium, another co-activator of classical PKCs. Others have suggested that palmitate incorporation into diacylglycerol rather than triacylglycerol is associated with apoptosis of MDA-MB-231 breast cancer cells (49). More recently, a possible role of PKC in stearate-induced apoptosis of breast cancer cells in vitro was investigated and it was found that stearate appears to work specifically via a diacylglycerol/PKC/caspase-3-mediated pathway (27). Although potentially this is a very important finding, it should be kept in mind that this mechanism has not yet been demonstrated in vivo with dietary stearate. In addition, although it may be related to the reduction of tumor burden seen in this and one other study (50), PKC pathways have also been shown to be involved in tumorigenesis. In fact, it has been suggested that Ras and PKC may cooperate during transformation (51). Investigation of a dietary stearate-induced link between PKC and Ras in the NMU carcinogen model was not explored in the present study but is a logical future study. Interestingly, in a transgenic model of colon cancer, transformation was found to be mediated by a PKCbII-Ras-PKCi-RAC1 (a Rho family member)-mitogen-activated protein kinase pathway that was found to be highly sensitive to a mitogen-activated protein kinase inhibitor (51). Thus, it is possible that a PKC/diacylglycerol/Rho signaling pathway mediates the effects of dietary stearate on carcinogenesis.

Nevertheless, results of the in vivo studies herein support our in vitro findings via inhibition of mammary tumor burden and carcinogenesis. Although one study has also found that stearate inhibits carcinogenesis using the NMU model (3), they injected iodostearic acid subcutaneously rather than give highly purified stearate in dietary form. The present study not only provides molecular insights as to how stearate is working but also shows for the first time that dietary stearate inhibits carcinogenesis. Recent studies have also indicated that dietary stearate inhibits breast cancer tumor and metastasis burden in an orthotopic nude mouse model (50). These studies indicate that dietary stearate may be a preventative agent, but is there evidence to support this role? Many case control and cohort studies have been performed in different countries to determine the correlation of dietary fat intake and breast cancer risk. Five meta-studies have summarized these results over the years and their results are conflicting. The link between total dietary fat intake and human breast cancer seems weak and may be related to menopausal hormone use (52). With respect to saturated fatty acids, three of these meta-studies found no association between saturated fatty acids and breast cancer (53-55) whereas two studies did (56, 57). Data obtained by actually measuring individual fatty acid composition of adipose tissue, erythrocyte membranes, serum and plasma provide quantitative measurement independent of energy intake, and reflects bioavailable and post-absorptive amounts of fat consumed. This eliminates inadequacies of food frequency questionnaires, food composition tables and nutrient databases. A meta-analysis of these data and the risk of breast cancer (3 cohort and 7 case-control studies with 2031 breast cancer cases and 2334 controls) indicate that in cohort studies, stearate was not associated with increased risk of breast cancer whereas palmitate was (58). They also demonstrate that in a cohort of post-menopausal women both stearate and the stearate/oleate ratio were negatively associated with breast cancer risk. This is consistent with a protective effect of stearate with respect to the risk of breast cancer. In this meta-analysis, no significant associations were derived from the case-control studies. Since the meta-analysis report, a case-control study looking at red blood cell fatty acids and breast cancer found that stearate did not have a positive association with breast cancer whereas palmitate did (59) similar to the results found from the cohort studies mentioned above. The only other such study since the meta-analysis was done found no relations between breast cancer risk and any fatty acids of erythrocyte membranes (60). Overall, these studies suggest that stearate is either neutral or may be protective for breast cancer and thus do not contraindicate a possible role for stearate in preventing breast cancer especially in post-menopausal women.

A limitation of these studies is that the in vitro experiments were only done on one cell line. The choice of Hs578t cells was made because initially we were interested in studying EGFR expressing breast cancer cells since the presence of the EGFR is associated with poorer outcomes. In addition, there remains a great need for therapies/prevention of this type of breast cancer exactly because it is so aggressive. Nevertheless, it is clear that stearate has affects on cancer cells other than basal breast cancer cells. The effects of stearate on HT1080 (human fibrosarcoma) and PC3 and DU145 (human prostate cancer) cells (61) have been published. Interestingly, the basal type non-tumorigenic but EGF-responsive breast cancer cell line MCF10A was not affected by stearate whereas Hs578t, MDA-MB435 and MDA-MB-231 cells were (27). Since estrogen suppresses the expression of the EGFR (62), we have yet to investigate estrogen-responsive (ER+) cell lines. Nevertheless, it is possible that stearate has similar effects on other cancer cell lines.

In summary, these in vitro results demonstrate that stearate inhibits breast cancer cell cycle largely in G₁, as well as inhibiting Rho expression and activity in vitro and expression in vivo. In vivo results further showed that dietary stearate inhibited the incidence and tumor burden of NMU-induced mammary cancer. These studies raise the possibility of stearate inhibiting Ras/Rho signaling and demonstrate the effectiveness of dietary stearate as a preventative agent for mammary cancer carcinogenesis.

### REFERENCES

1 Wickramasinghe, N. S. et al. (1996) Stearate inhibition of breast cancer cell proliferation. Am. J. Pathol., 148, 987-995.
2 Hardy, R. W. et al. (1997) Fatty acids and breast cancer cell proliferation. Adv. Exp. Med. Biol., 422, 57-69.
3 Habib, N. A. et al. (1987) Stearic acid and carcinogenesis. Br. J. Cancer, 56, 455-458.
4 Welsch, C. W. (1992) Relationship between dietary fat and experimental mammary tumorigenesis: a review and critique. Cancer Res., 52, 2040-2048.
5 Lui, V. W. et al. (2002) EGFR-mediated cell cycle regulation. Anticancer Res., 22, 1-11.
6 Mansour, E. G. et al. (1994) Prognostic factors in early breast carcinoma. Cancer, 74, 381-400.
7 Klijn, J. G. et al. (1992) The clinical significance of epidermal growth factor receptor (EGF-R) in human breast cancer: a review on 5232 patients. Endocrinol. Rev., 13, 3-17.
8 Threadgill, D. W. et al. (1995) Targeted disruption of mouse EGF receptor: effect of genetic background on mutant phenotype. Science, 269, 230-234.
9 Takai, Y. et al, (2001) Small GTP-binding proteins. Physiol. Rev., 81, 153-208.
10. Malaney, S. et al. (2001) The Ras signaling pathway in mammary tumorigenesis and metastasis. J. Mammary Gland Biol. Neoplasia, 6, 101-113.
11.Pruitt, K. et al. (2001) Ras and Rho regulation of the cell cycle and oncogenesis. Cancer Lett., 171, 1-10.
12. Sahai, E. et al. (2001) Cross-talk between Ras and Rho signaling pathways in transformation favors proliferation and increased motility. EMBO J., 20, 755-766.
13. Spector, A. A. et al. (1969) An improved method for the addition of long chain fatty acid to protein solution. Anal. Biochem., 32, 297-302.
14. Sherr, C. J. et al. (1999) CDK inhibitors: positive and negative regulators of G1-phase progression. Genes Dev., 13, 1501-1512.
15.Toyoshima, H. et al. (1994) p27KIP1, a novel inhibitor of G1 cyclin-Cdk protein kinase activity, is related to p21CIP1/WAF1. Cell, 78, 67-74.
16. Gille, H. et al. (1999) Multiple ras effector pathways contribute to G(1) cell cycle progression. J. Biol. Chem., 274, 22033-22040.
17. Suwa, H. et al. (1998) Overexpression of the RhoC gene correlates with progression of ductal adenocarcinoma of the pancreas. Br. J. Cancer, 77, 147-152.
18. Fritz, G. et al. (1999) Rho GTPases are over-expressed in human tumors. Int. J. Cancer, 81, 682-687.
19. Kamai, T. et al. (2001) Overexpression of RhoA mRNA is associated with advanced stage in testicular germ cell tumor. BJU Int., 87, 227-231.
20. Kleer, C. G. et al. (2002) Characterization of RhoC expression in benign and malignant breast disease: a potential new marker for small breast carcinomas with metastatic ability. Am. J. Pathol., 160, 579-584.
21.van Golen, K. L. et al. (1999) A novel putative low-affinity insulin-like growth factor-binding protein, LIBC (lost in inflammatory breast cancer), and RhoC GTPase correlate with the inflammatory breast cancer phenotype. Clin. Cancer Res., 5, 2511-2519.
22. Olson, M. F. et al. (1998) Signals from Ras and Rho GTPases interact to regulate expression of p21CIP1/WAF1. Nature, 394, 295-299.
23. Chan, M. M. et al. (2005) Gene expression profiling of NMU-induced rat mammary tumors: cross species comparison with human breast cancer. Carcinogenesis, 8, 1343-1353.
24. Mu, Y. M. et al. (2001) Saturated FFAs, palmitic acid and stearic acid, induce apoptosis in human granulosa cells. Endocrinology, 142, 3590-3597.
25. Artwohl, M. et al. (2004) Free fatty acids trigger apoptosis and inhibit cell cycle progression in human vascular endothelial cells. FASEB J., 18, 146-148.
26. Hardy, S. et al. (2003) Saturated fatty acid-induced apoptosis in MDAMB-231 breast cancer cells. J. Neurochem., 84, 655-668.
27. Evans, L. M. et al. (2009) Stearate preferentially induces apoptosis in human breast cancer cells. Nutr. Cancer, 61, 746-753.
28. Hunnicutt, J. W. et al. (1994) Saturated fatty acid-induced insulin resistance in rat adipocytes. Diabetes, 43, 540-545.
29. Reaven, G. M. (1999) The pathological consequences of adipose tissue insulin resistance. In Reaven, G. M. and Laws, A. (eds.) Insulin Resistance, The Metabolic Syndrome X. Humana Press, Totowa, N.J., pp. 242-244.
30. Xiong, Y. et al. (1993) p21CIP1/WAF1 is a universal inhibitor of cyclin kinases. Nature, 366, 701-704.
31. Hunter, T. et al. (1994) Cyclins and cancer. II: cyclin D and CDK inhibitors come of age. Cell, 79, 573-582.
32. Sheaff, R. J. et al. (1997) Cyclin E-CDK2 is a regulator of p27KIP1. Genes Dev., 11, 1464-1478.
33. Vlach, J. et al. (1997) Phosphorylation-dependent degradation of the cyclindependent kinase inhibitor p27KIP1. EMBO J., 16, 5334-5344.
34. Khosravi-Far, R. et al. (1995) Activation of Rac1, RhoA, and mitogen-activated protein kinases is required for Ras transformation. Mol. Cell. Biol., 15, 6443-6453.
35. Qiu, R. G. et al. (1995) An essential role for Rac in Ras transformation. Nature, 374, 457-459.
36. Qiu, R. G. et al. (1997) Cdc42 regulates anchorage-independent growth and is necessary for Ras transformation. Mol. Cell. Biol., 17, 3449-3458.
37. Bodemann, B. O. et al. (2008) GTPases and cancer: linchpin support of the tumorigenic platform. Nat. Rev. Cancer, 8, 133-140.
38. Narumiya, S. et al. (2009) Rho signaling, ROCK and mDial, in transformation, metastasis and invasion. Cancer Metastasis Rev., 28, 65-76.
39. Coleman, M. L. et al. (2003) Ras promotes p21CIP1/WAF1 protein stability via a cyclin D1-impopsed block in proteasome-mediated degradation. EMBO J., 22, 2036-2046.
40. Chen, J. C. et al. (2003) Oncogenic Ras leads to Rho activation by activating the mitogen activated protein kinase pathway and decreasing Rho-GTPase-activating protein activity. J. Biol. Chem., 278, 2807-2818.
41. Wu, M. et al. (2004) RhoC induces differential expression of genes involved in invasion and metastasis in MCF10A breast cells. Breast Cancer Res. Treat., 84, 3-12.
42. Kamai, T. et al. (2003) Significant association of Rho/ROCK pathway with invasion and metastasis of bladder cancer. Clin. Cancer Res., 9, 2632-2641.
43. Horiuchi, A. et al. (2003) Up-regulation of small GTPases, RhoA and RhoC, is associated with tumor progression in ovarian carcinoma. Lab. Invest, 83, 861-870.
44. Clark, E. A. et al. (2000) Genomic analysis of metastasis reveals an essential role for RhoC. Nature, 406, 532-535.
45. Kito, K. et al. (1996) Incidence of p53 and Ha-ras gene mutations in chemically induced rat mammary carcinomas. Mol. Carcinog., 17, 78-83.
46. Sukumar, S. et al. (1995) Animal models for breast cancer. Mutat. Res., 333, 37-44.
47. Awad, A. B. et al. (1996) Effect of membrane lipid alteration on the growth, phospholipase C activity and G protein of HT-29 tumor cells. Prostaglandins Leukot. Essent. Fatty Acids, 55, 293-302.
48. Hanahan, D. J. (1997) Phospholipid Chemistry. Oxford University Press, New York, N.Y., 20-149.
49. Hardy, S. et al. (2003) Saturated fatty acid-induced apoptosis in MDAMB-231 breast cancer cells: a role for cardiolipin. J. Biol. Chem., 278, 31861-31870.
50. Evans, L. M. et al. (2009) Dietary stearate reduces human breast cancer metastasis burden in athymic nude mice. Clin. Exp. Metastasis, 26, 415-424.
51. Griner, E. M. et al. (2007) Protein kinase C and other diacylglycerol effectors in cancer. Nat. Rev. Cancer, 7, 281-294.
52. Smith-Warner, S. A. et al. (2007) Fat intake and breast cancer revisited. J. Natl. Cancer Inst., 99, 418-419.
53. Boyd, N. F. et al. (1993) A meta-analysis of studies of dietary fat and breast cancer risk. Br. J. Cancer, 68, 627-636.
54. Byers, T. (1994) Nutritional risk factors for breast cancer. Cancer, 74, 288-295.
55. Hunter, D. J. et al. (1996) Cohort studies of fat intake and the risk of breast cancer—a pooled analysis. N. Engl. J. Med., 334, 356-361.
56. Boyd, N. F. et al. (2003) Dietary fat and breast cancer risk revisited: a metaanalysis of the published literature. Br. J. Cancer, 89, 1672-1685.
57. Howe, G. R. et al. (1990) Dietary factors and risk of breast cancer: combined analysis of 12 case-control studies. J. Natl. Cancer Inst., 82, 561-569.
58. Saadatian-Elahi, M. et al. (2004) Biomarkers of dietary fatty acid intake and the risk of breast cancer: a meta-analysis. Int. J. Cancer, 111, 584-591.
59. Shannon, J. et al. (2007) Erythrocyte fatty acids and breast cancer risk: a case-control study in Shanghai, China. Am. J. Clin. Nutr., 85, 1090-1097.
60. Wirfalt, E. et al. (2004) No relations between breast cancer risk and fatty acids of erythrocyte membranes in postmenopausal women of the Malmo Diet Cancer Cohort (Sweden). Eur. J. Clin. Nutr., 58, 761-770.
61. Singh, R. K. et al. (1995) Stearate inhibits human tumor cell invasion. Invasion Metastasis, 15, 144-155.
62. Yarden, R. I. et al. (2001) Estrogen suppression of EGFR expression in breast cancer cells: a possible mechanism to modulate growth. J. Cell. Biochem., 36 (suppl), 232-246.

### Example 3

### Stearate as an Adjuvant for Paclitaxel

Chemotherapy with paclitaxel (PTX) and other taxanes are considered fundamental drugs in the treatment of breast cancer. However, due to the severe side effects, identification of effective adjuvant therapies to paclitaxel is needed. Stearate is an 18-carbon saturated fatty acid found in many foods in the Western diet. It has been shown to have anti-cancer properties during early stages of neoplastic progression. The previous study demonstrated that dietary stearate reduces human breast cancer metastasis burden in athymic nude mice, and suggested the possibility of dietary stearate as a potential adjuvant therapeutic strategy for breast cancer patients. In this study, the anti-metastatic effect of dietary stearate investigated was investigated in the presence of paclitaxel chemotherapy and its interaction with paclitaxel. Dietary stearate dramatically reduces the incidence and the number of lung metastasis in breast cancer mouse model when it was initiated before cancer cell injection or after the primary tumor is removed. The effect of dietary stearate and paclitaxel is additive. Inhibition of angiogenesis may be the main mechanism of this adjuvant effect of stearate. Overall, this study suggests that the combination of dietary stearate with paclitaxel chemotherapy merits further investigation for breast cancer treatment.

### Materials and Methods

Animals and Diets

3-4 week old female athymic mice were purchased from Harlan Sprague Dawley, Inc. (Indianapolis, Ind.) and were maintained in microisolater cages in pathogen-free facilities. Four kinds of diets were used in our experiment: a control (low fat) diet (5% corn oil) comparable to normal rodent chow, a safflower oil diet (20% safflower oil), a corn oil diet (17% corn oil/3% safflower oil) and a stearate diet (17% stearate/3% safflower oil). The diets were prepared by Harlan-Teklad (Madison, Wis.). The animals were fed ad libitum and the amount of food consumed was recorded. Mice were anesthetized with 3% isoflurane in 2.5% O₂ and weighed weekly. All in vivo procedures were approved by the Institutional Animal Care and Use Committee (IACUC), University of Alabama at Birmingham (UAB).

Cancer Cells

MDA-MB-435 human breast cancer cells (obtained from Dr. Dan Welch; UAB) were grown and maintained in DMEM:F12 supplemented with 5% FBS, 2 mM glutamine, 1 mM sodium pyruvate, 0.2× non-essential amino acids and 1% penicillin/streptomycin (5% CO₂). Cells were grown to 80-90% confluence prior to preparation for injection. To detach cells from the plates, cells were washed with PBS and then treated with 3 mM versene. Cells were pelleted by centrifugation and resuspended in Hank's buffered saline solution (HBSS). Cells were diluted to 10⁷ cells/ml and were kept on ice until the time of injection to prevent clumping.

Experimental Design

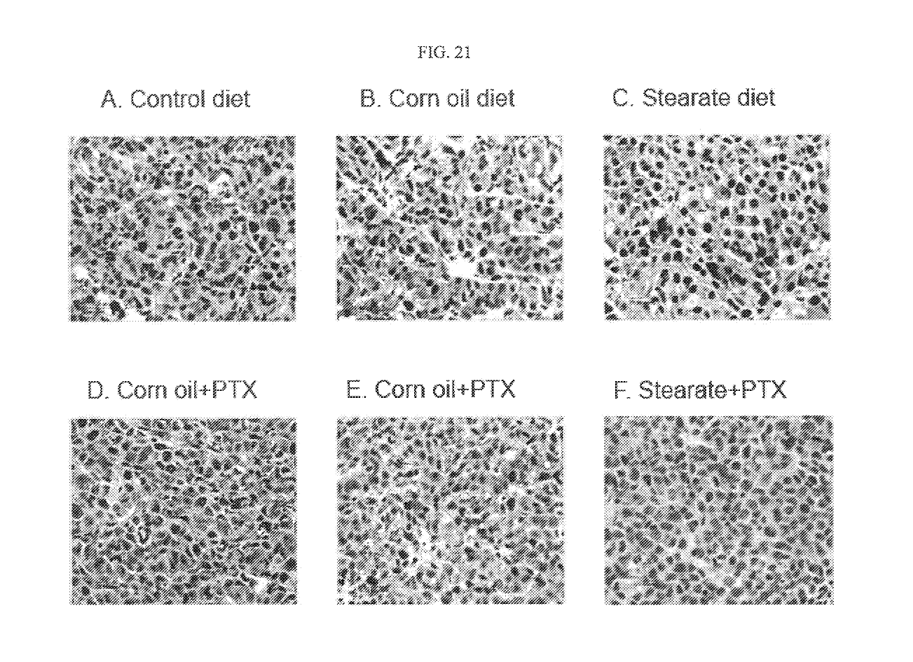

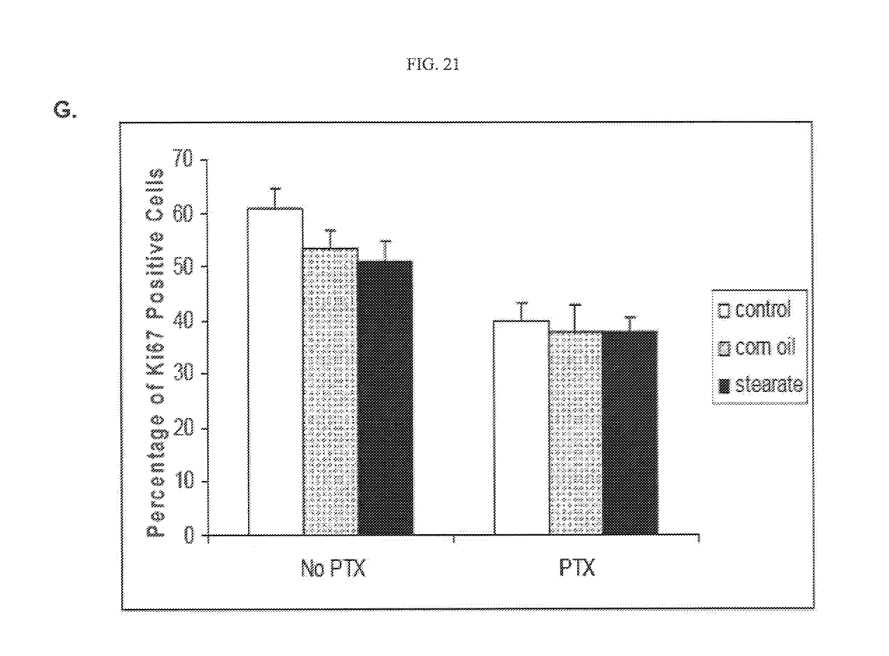

The experimental timetable is shown in FIG. 21. Briefly, in experiment 1, animals were divided randomly into one of four groups—a control diet group, a corn oil diet group, a safflower oil diet group, and a stearate diet group. All animals were placed on the diets 3 weeks prior to injection of cancer cells. The tumors were allowed to reach an approximate mean tumor diameter of 10-12 mm (253.6-904.8 mm³) at which time the primary tumors were removed (≦9 weeks post-injection). Chemotherapy with paclitaxel started 1 week after the surgery. After that, the animals were allowed to develop metastases for about 4 weeks, sacrificed and the lungs were collected. In experiment 2, diet therapy was initiated at the same time as chemotherapy, about 1 week after surgery.

Before diet therapy, the mice were feed with control diet. The mice were divided into six groups evenly according to the size of primary tumor—a control diet group, a corn oil diet group, a stearate diet groups, a control diet plus PTX group, a corn oil diet plus PTX group, and a stearate diet plus PTX groups. All in vivo procedures were approved by the institutional animal care and use committee.

Mammary Fat Pad Injections

Animals were anesthetized with 3% isoflurane in 2.5% O₂. The right chest skin was cleaned with a betadine solution. A small incision was made between the right 2^{nd} and 3^{rd} mammary fat pads and 10⁶ MDA-MB-435 cells suspended in HBSS were injected into the 2nd mammary fat pad using a 27 mm gauge needle (final volume of 100 μl). A single wound clip was used to close the incision and removed the following week.

Paclitaxel Intraperitoneal Injections

Paclitaxel (PTX) from LC Laboratories (Woburn, Mass.) was dissolved in Cremophor EL:ethanol (1:1, v:v) and then diluted with sterile physiological saline to a final concentration of 0.5 mg/ml. The drug dosage for this experiment is about 20 mg/kg.

The animals were anesthetized with isoflurane. The abdominal skin was cleaned with a betadine solution. One ml of above paclitaxel solution was injected intraperitoneally.

Tumor Measurement

After the injection of the cells, mice were monitored weekly for the development of primary tumor masses. Once the tumors became visible (1-2 weeks post-injection), they were measured using a digital caliper. The tumor volume was estimated using the equation for a prolate ellipsoid where volume=π (4/3) (length/2) (width/2) [(length+width)/4].

Tumor Excision

The animals were anesthetized with isoflurane. The skin overlying the mammary tumor area was cleaned with a betadine solution and an incision was made circumferentially around the tumor down to its base. The wound was closed using wound clips which were removed 1 week later.

Necropsy

At the end of the experiment, mice were anesthetized with a combination of ketamine and xylazine and then decapitated. The lungs were dissected from the mice and stored in formalin prior to the counting of visible tumors on all surfaces of the lungs. Two examiners did the counting separately. The examiners were blinded to the identity of the samples prior to counting. The average of the data from both examiners is used for analysis.

Lung Metastatic Tumor Size Measurement

Lungs were placed under the dissecting microscope (Fisher Scientific, Hanover Park, Ill.) for measurement of metastatic tumor size. Tumor size is expressed as an average of the longest and shortest diameter. The tumors were split into three groups according to their sizes (<0.1 cm, small tumor; 0.1-0.2 cm, medium tumor; >0.2 cm, large tumor), and the number of tumors per mouse was counted separately.

Immunohistochemistry

Paraffin sections were prepared as described previously (21). 5 μm thick sections were cut from the formalin fixed, paraffin embedded tissue blocks and floated onto charged glass slides (Super-Frost Plus, Fisher Scientific, Pittsburgh, Pa.) and dried overnight at 60° C. A hemotoxylin and eosin stained section was obtained from each tissue block. All sections for immunohistochemistry were deparaffinized and hydrated using graded concentrations of ethanol to deionized water.

CD31 immunostaining was done as described previously⁽²¹⁾. Briefly, the tissue sections were subjected to pretreatment with 0.5 M tris buffer (pH 10). All sections were washed gently in deionized water, then transferred in to 0.05 M Tris-based solution in 0.15 M NaCl with 0.1% v/v Triton-X-100, pH 7.6 (TBST). Endogenous peroxidase was blocked with 3% hydrogen peroxide for 10 min. To reduce further nonspecific background staining, slides were incubated with avidin (Jackson ImmunoResearch, West Grove, Pa.) and biotin blocking solutions (Sigma, to St. Louis, Mo.) for 15 min each, and 3% normal goat serum (Sigma, St. Louis, Mo.) for 20 min. All slides were then incubated at 4° C. overnight with rabbit polyclonal antibody against CD31 (1:200 dilution) (Abeam, Cambridge, Mass.). After washing with TBST, biotinylated goat anti-rabbit IgG (1:1000; Jackson ImmunoResearch, West Grove, Pa.) were applied to the sections for 30 min at room temperature. Sections were then incubated with Strepavidin-HRP (Sigma, St. Louis, Mo.) for 30 min at room temperature. Diaminobenzidine (DAB; Scy Tek Laboratories, Logan, Utah) was used as the chromagen and hematoxylin (Richard-Allen Scientific, Kalamazoo, Mich.) as the counterstain.

Ki67 and caspase-3 immunostaining were done according to the protocol from Cell Signaling. Briefly, the tissue sections were subjected to pretreatment with 0.01 M sodium citrate buffer (pH 6). All sections were washed gently in deionized water, and then transferred in to TBST. Endogenous peroxidase was blocked with 3% hydrogen peroxide for 10 min. To reduce further nonspecific background staining, slides were incubated with 3% normal goat serum for 1 hour. All slides were then incubated at 4° C. overnight with rabbit monoclonal antibody against cleaved caspase-3 (1:200 dilution, Cell Signaling, Danvers, Mass.) or rabbit polyclonal antibody to Ki67 (1:200, Abcam, Cambridge, Mass.). After washing with TBST, SignallStain Boost IHC Detection Reagent (Cell Signaling, Danvers, Mass.) were applied to the sections for 30 min at room temperature. Diaminobenzidine was used as the chromagen and hematoxylin as the counterstain. Negative control was produced by eliminating the primary antibody from the diluents.

Bioquant® Image Analysis software (Rtm Biometrics, Nashville, Tenn.) was used to evaluate the immunostaining. For Ki67 and caspase-3 immunostaining, three hot spots (1 μM²) were selected at magnification X4. The numbers of positive and negative cells in these hot spots were then counted and averaged at 5 magnification X40. The percentage of cells stained with Ki67 or caspase-3 was subsequently calculated for comparison. For CD31 immunostaining, the MVD was measured based on Weidner's method (37). Briefly, three hot spots (4 μM2) were selected at magnification X10. The number of microvessels in these hot spots was counted at magnification X40, and the density was then calculated for comparison. Each positive endothelial cell cluster of immunoreactivity was counted as an individual vessel in addition to the morphologically identifiable vessels with a lumen.

### Statistical Analysis

Data were presented as the mean±SEM. SigmaStat 3.1 software program was used for statistics. The statistical comparisons of the number of lung metastasis were performed by one-way analysis of variance (ANOVA) with the Holm-Sidak test. Two-way ANOVA was used to examine the interaction between chemotherapy and diet therapy. We used Chi-square to evaluate the incidence of lung metastasis. The significant differences were indicated as p<0.05.

### Results

Food Intake and Weight Gain

Since low fat (control), safflower oil, corn oil and stearate diets are not isocaloric, food consumption and weight gain were monitored to ensure the animals did not have significant discrepancies in energy intake. In our experiment, the control diet mice consumed the most kilocalories/day (0.98 kcal/day), followed by the stearate diet animals (0.80 kcal/day), and then the corn oil and safflower oil diet ingesting mice (0.69 kcal/day). Despite differences in food intake, there was no overt difference in weight gain between the diets (data not shown here).

Early Initiation of Dietary Stearate Reduced the Incidence and the Number of Lung Metastasis

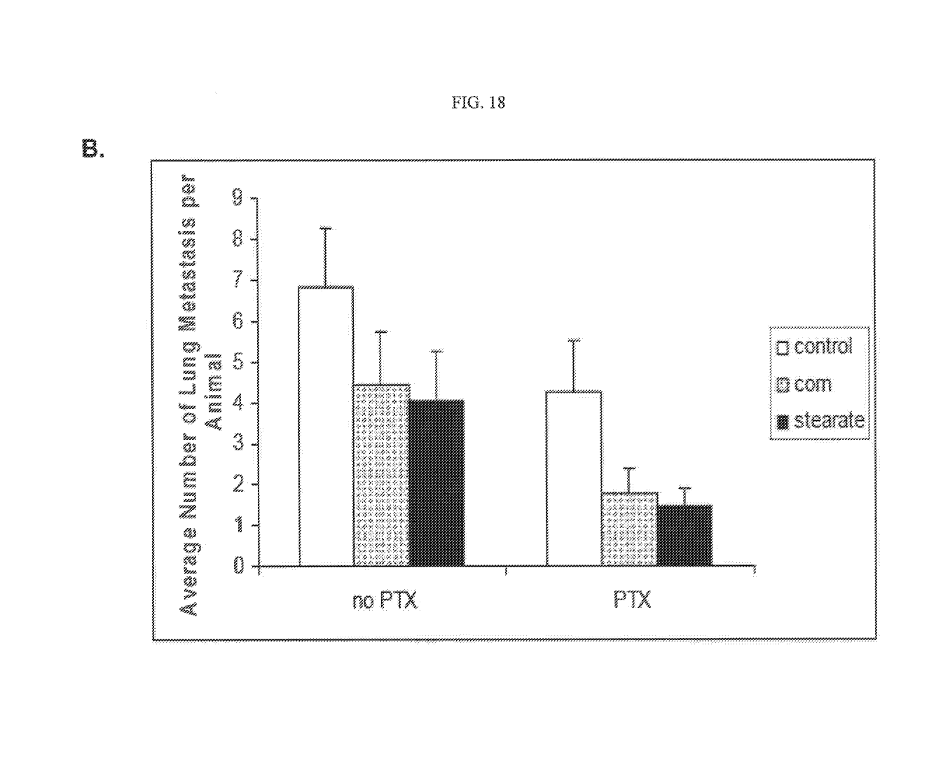

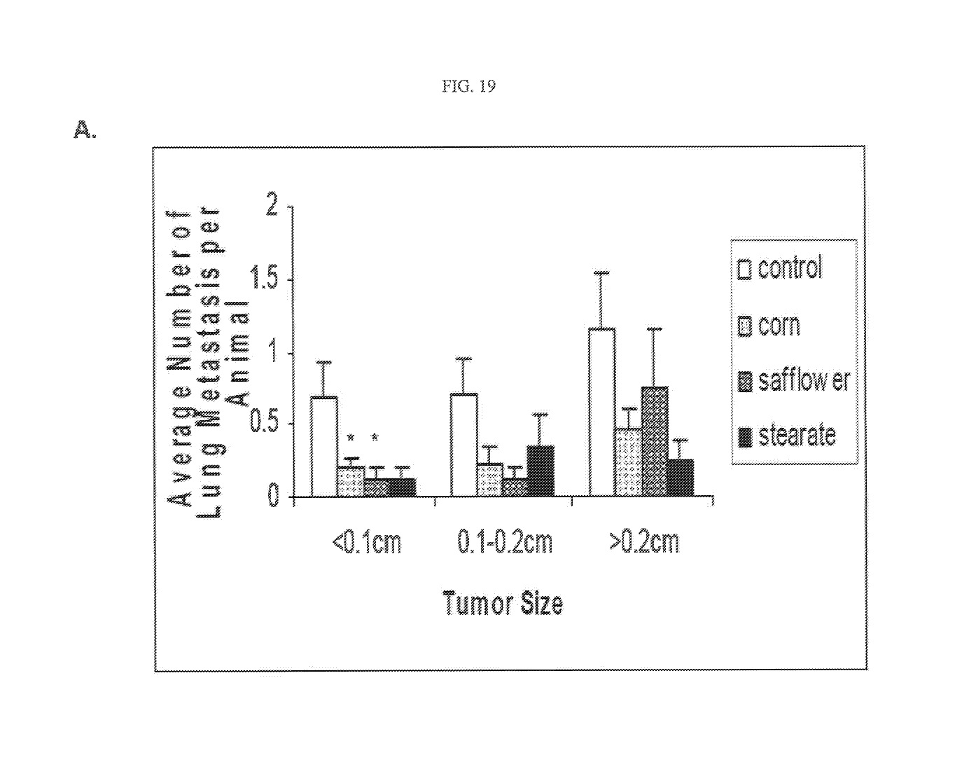

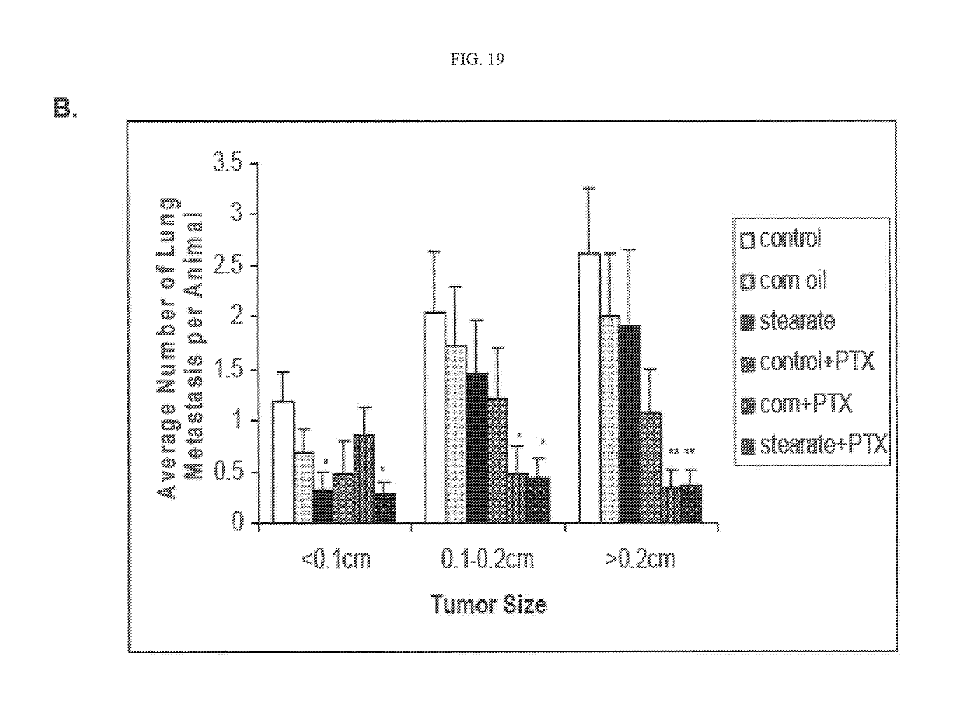

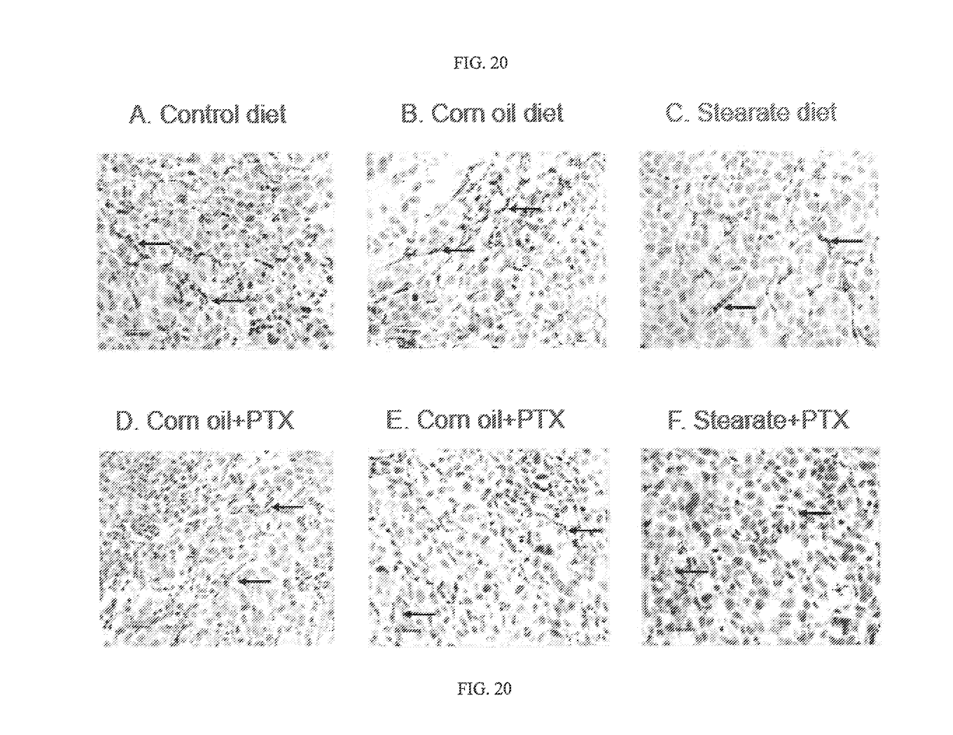

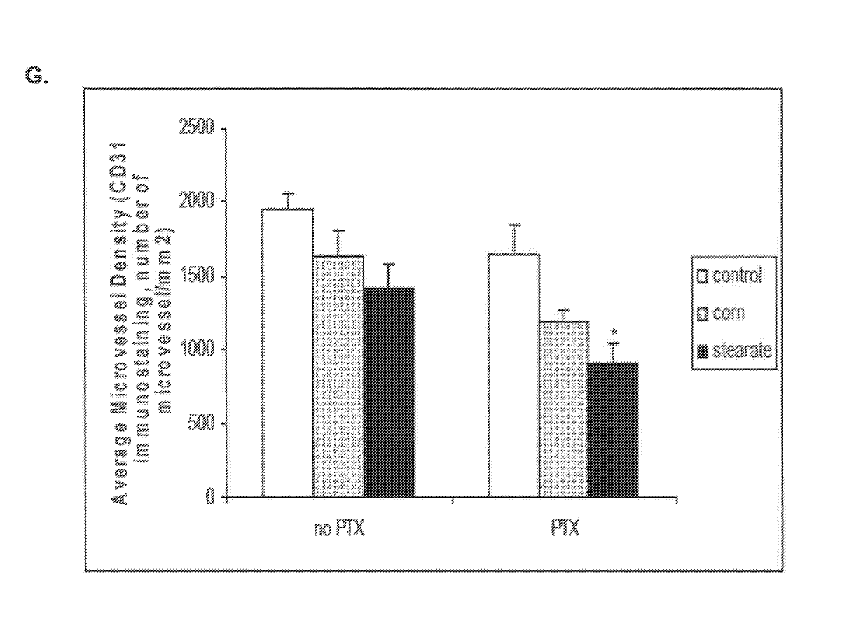

In experiment 1, diet therapy was initiated 3 weeks before breast cancer cell injection. As shown in FIG. 18 A, mice on stearate diet had significantly decreased incidence of lung metastasis compared to the control and corn oil diet groups. Mice on stearate diet also had significantly reduced number of lung metastases compared to those on control diet (FIG. 19 A). When the size of lung metastatic tumors was measured, the data showed that mice on safflower oil and stearate diet had fewer small size lung metastases (FIG. 20 A).

Late Initiation of Dietary Stearate Also Decreased the Incidence and the Number of Lung Metastasis, which is Additive to Chemotherapy

In experiment 2, diet therapy was initiated 1 week after the primary tumors were removed, the same time as chemotherapy. As shown in FIG. 18 B, mice on both stearate and corn oil diet had significantly decreased incidence of lung metastases compared to the control diet group. Mice on chemotherapy had lower incidence of lung metastases in different diet conditions.

When the number of metastatic tumors per animal was counted and compared, two-way ANOVA showed that both chemotherapy and diet stearate significantly reduced the number of lung metastases (FIG. 19 B). The effect of chemotherapy and dietary stearate is considered to be additive since their interaction is not synergistic statistically.

As shown in FIG. 20 B, mice on corn oil diet plus PTX and stearate diet plus PTX had significantly decreased number of medium and large size lung metastasis. The number of small size tumors also significantly decreased in stearate diet and stearate diet plus PTX groups.

Paclitaxel Chemotherapy and Stearate Diet Inhibit Angiogenesis of Metastatic Tumors

CD31 immunostaining is used in our experiment to quantify tumor angiogenesis. As shown in FIG. 21 A-F, tumors from the stearate diet groups and paclitaxel chemotherapy groups have reduced number of microvessels. Two-way ANOVA verified that both chemotherapy and diet therapy affected the microvessel density (MVD) significantly. Further analysis showed that tumors from stearate diet group had significantly reduced MVD in the presence of chemotherapy (FIG. 21 G).

The Effect of Paclitaxel Chemotherapy and Diet Therapy on Proliferation

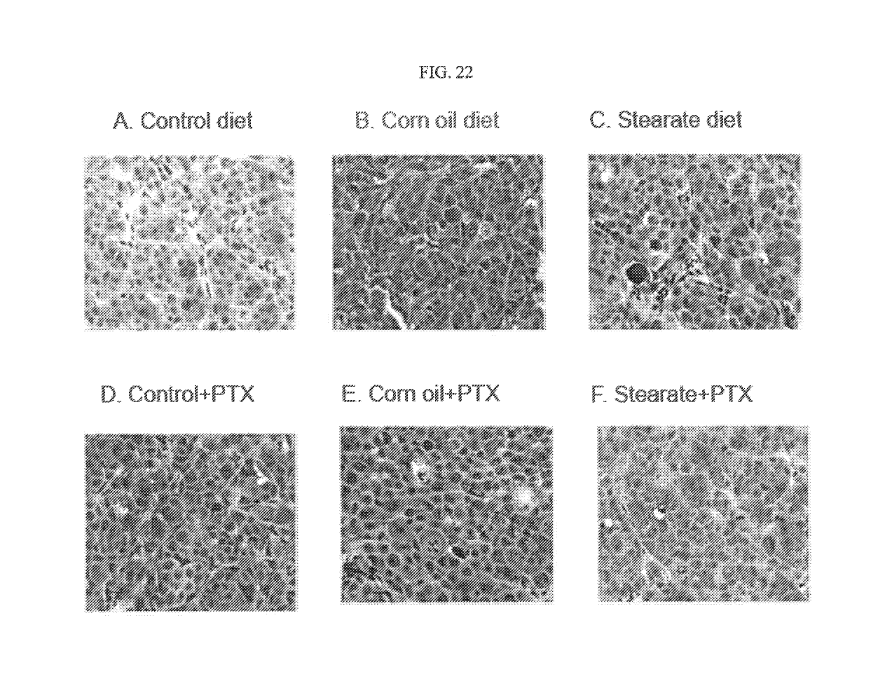

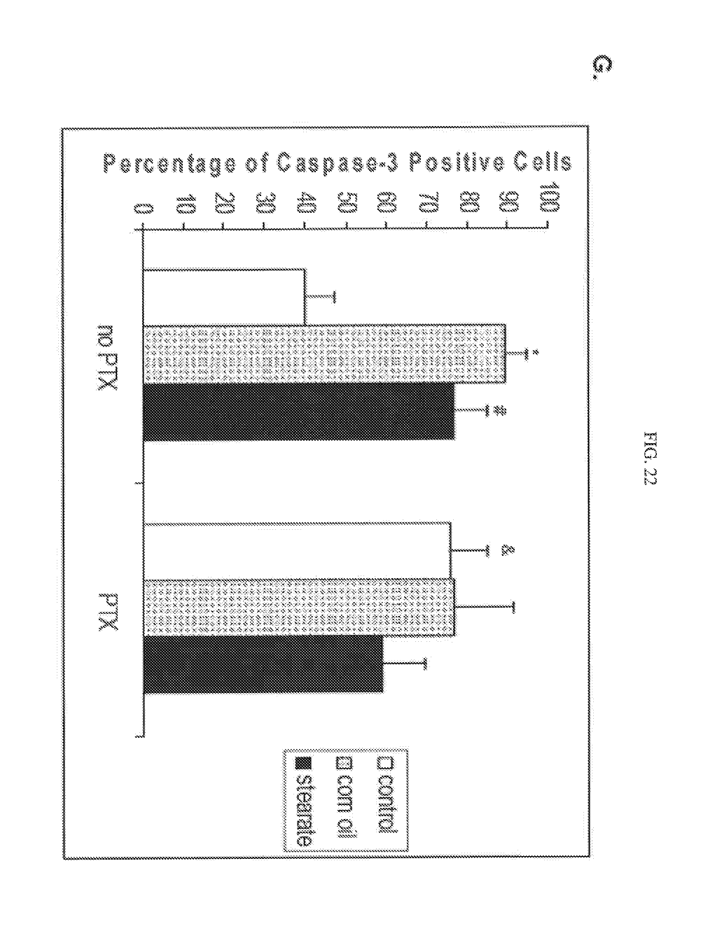

With Ki67 immunostaining, we investigated the effect of chemotherapy and diets on proliferation. As shown in FIG. 22 A-F, tumors from chemotherapy groups had overt fewer Ki67 positive cells. Two-way ANOVA showed that chemotherapy significantly decreased the percentage of Ki67 positive cells, while diets did not (FIG. 22 G).

The Effect of Chemotherapy and Diet Therapy on Apoptosis

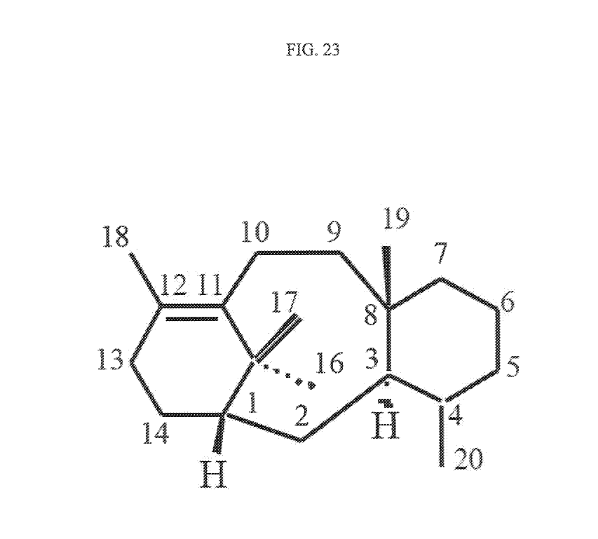

Caspase-3 immunostaining was used in this experiment to evaluate the apoptosis of metastatic tumors. As shown in FIG. 23 A-G, tumors from stearate and corn oil diet groups had significantly more caspase-3 positive cells. However, in the presence of chemotherapy, no difference was observed among these three diet groups.

### Discussion

Stearate has been found to inhibit proliferation, inhibit invasion, inhibit cell cycle and induce apoptosis of breast cancer and other cells. Its “anticancer” properties range from prevention of carcinogenesis, inhibition of breast cancer tumor burden and reduction of human breast cancer metastasis (5, 6, 7, 11, 20, 22). In the present experiment, it was demonstrated the possibility of stearate functioning as an adjuvant of paclitaxel chemotherapy. This is the first study to investigate the interaction of dietary stearate and paclitaxel chemotherapy in vivo. The significance of these studies comes from the potential clinical applications of stearate in the treatment of breast cancer. Breast cancer is the most frequently diagnosed cancer and the leading cause of cancer death in females worldwide (1). Chemotherapy with paclitaxel and other taxanes are considered fundamental drugs in the treatment of breast cancer besides surgery. However, due to the severe side effects, identification of effective adjuvant therapies to paclitaxel is needed. In this experiment, the early initiation of dietary stearate before the injection of breast cancer cells mimics the clinical situation that a female patient starts stearate therapy preventively before she is found to have breast cancer. The late initiation of dietary stearate after the primary breast cancer removal mimics the clinical situation that a female patient starts stearate therapy and chemotherapy after surgery.

Unlike other saturated fatty acids, such as palmitate (C16:0), stearate does not increase plasma low density lipoprotein cholesterol concentrations (4). According to a recent review (36) of epidemiologic and clinical studies that evaluated the relation between stearate and cardiovascular disease risk factors, the adverse affect of stearate is limited compared with cholesterol-raising saturated fatty acid (SFA) and trans fatty acid (TFA). Therefore, stearate is being evaluated as a substitute for SFA and TFA in food manufacturing. The unique anti-cancer properties demonstrated recently and in this experiment encourage the increased use of stearate in food supply and diet.

Epidemiological and animal studies have demonstrated the protective effect of stearate with respect to the risk of breast cancer. A cohort study of post-menopausal women showed that both stearate and stearate/oleate ratio were negatively associated with breast cancer risk (38). In recent experiments, dietary stearate was used in the NMU rat breast cancer carcinogen model, and found that stearate reduces the incidence of NMU induced mammary cancer and the tumor burden (22). But, this anti-carcinogenesis effect of stearate is not involved in the anti-metastatic effect demonstrated in the present experiment, since the breast cancer cells were injected. In another study, evidence was presented that dietary stearate inhibited the growth of MDA-MB-435 human breast cancer cells in the mammary fat pad model system and partially reduced metastatic burden in the lungs. Further experiments showed that the inhibition of metastasis was independent of the size of primary tumor as animals that developed larger tumors also had an inhibition of metastasis (11). Therefore, the anti-metastasis of stearate might be an independent effect.

The primary mechanism of action of taxanes is to stabilize microtubules and prevent their disassembly (3). Studies (14-18) showed that paclitaxel is an inhibitor of angiogenesis and proliferation, and an inducer of apoptosis in some cancer diseases including breast cancer. The mechanisms in which paclitaxel and stearate interact are still elusive. Recent studies have shown that without paclitaxel chemotherapy, the anti-metastasis effect of stearate may be due, at least in part, to the ability of stearate to induce apoptosis in these human breast cancer cells (11). However, in the presence of paclitaxel chemotherapy, the situation is complicated. Although dietary stearate itself significantly induced apoptosis, this effect becomes insignificant in the presence of paclitaxel. Therefore, stearate induced apoptosis does not add more anti-metastatic effect to paclitaxel, though it may be important without chemotherapy. Proliferation inhibition is another possible mechanism. Although inhibition of cancer cell proliferation was found to be an important effect of stearate (5, 22), its role in metastasis is not obvious. Our present experiment showed that the inhibition of angiogenesis may be an important mechanism. CD31 immunostaining is a widely used method to quantify tumor angiogenesis. The microvessel density calculated according to CD31 staining were found significantly decreased in both paclitaxel and stearate treated mice, and their effect was additive. Further analysis showed that in the presence of chemotherapy, stearate significantly reduced angiogenesis additionally. This suggests the important role of angiogenesis inhibition in the anti-metastatic effect of stearate. One of the critical steps of angiogenesis is the proliferation of vascular endothelial cells (29), and it has been shown that angiogenesis may be inhibited by selective induction of apoptosis in proliferating endothelial cells (27, 28). Some experiments proved that stearate, time, and concentration dependently increased endothelial apoptosis (25, 26). Stearate induced endothelial apoptosis may be a reason that causes angiogenesis inhibition.

In summary, dietary stearate reduced breast cancer lung metastatic burden on the basis of chemotherapy whether it was initiated before cancer cell injection or after surgery. The inhibition of angiogenesis may be a potential related mechanism. These results suggest dietary stearate should be evaluated as an adjuvant with chemotherapy in clinical trials for breast cancer treatment.

### REFERENCES

1. Jemal A, Bray F, Center M M, Ferlay J, Ward E, Forman D. Global cancer statistics. CA Cancer J Clin. 2011 March-April; 61(2):69-90.
2. Saloustros E, Mavroudis D, Georgoulias V. Paclitaxel and docetaxel in the treatment of breast cancer. Expert Opin Pharmacother. 2008 October; 9(15):2603-16.
3. Pienta K. Preclinical mechanisms of action of docetaxel and docetaxel combinations in prostate cancer. Semin Oncol. 2001; 28 (4 Suppl 15):3-7.
4. Grundy S M. Influence of stearic acid on cholesterol metabolism relative to other long-chain fatty acids. Am J Clin Nutr. 1994; 60:986 S-990S
5. Wickramasinghe N S, Jo H, McDonald J M, Hardy R W. Stearate inhibition of breast cancer cell proliferation. A mechanism involving epidermal growth factor receptor and G-proteins. Am J Pathol. 1996; 148:987-995.
6. Hardy S, El-Assaad W, Przybytkowski E, Joly E, Prentki M, Langelier Y. Saturated fatty acid-induced apoptosis in MDA-MB-231 breast cancer cells. A role for cardiolipin. J Biol Chem. 2003; 278:31861-31870.
7. Singh R K, Hardy R W, Wang M H, Williford J, Gladson C L, McDonald J M, Siegal G P. Stearate inhibits human tumor cell invasion. Invasion Metastasis 1995; 15:144-155.
8. Tinsley I J, Schmitz J A, Pierce D A. Influence of dietary fatty acids on the incidence of mammary tumors in the C3H mouse. Cancer Res. 1981; 41:1460-1465.
9. Bennett A S. Effect of dietary stearic acid on the genesis of spontaneous mammary adenocarcinomas in strain A/ST mice. Int J Cancer. 1984; 34:529-533.
10. Habib N A, Wood C B, Apostolov K, Barker W, Hershman M J, Aslam M, Heinemann D, Fermor B, Williamson R C, Jenkins W E et al. Stearic acid and carcinogenesis. Br J Cancer. 1987; 56:455-458.
11. Evans L M, Toline E C, Desmond R, Siegal G P, Hashim A I, Hardy R W. Dietary stearate reduces human breast cancer metastasis burden in athymic nude mice. Clin Exp Metastasis. 2009; 26(5):415-24.
12. Townson J L, Naumov G N, Chambers A F. The role of apoptosis in tumor progression and metastasis. Curr Mol Med. 2003 November; 3(7):631-42.
13. Boedefeld W M 2nd, Bland K I, Heslin M J. Recent insights into angiogenesis, apoptosis, invasion, and metastasis in colorectal carcinoma. Ann Surg Oncol. 2003; 10(8):839-51.
14. Wang J, Lou P, Lesniewski R, Henkin J. Paclitaxel at ultra low concentrations inhibits angiogenesis without affecting cellular microtubule assembly. Anticancer Drugs. 2003 January; 14(1):13-9.
15. Lau D H, Xue L, Young L J, Burke P A, Cheung A T. Paclitaxel (Taxol): an inhibitor of angiogenesis in a highly vascularized transgenic breast cancer. Cancer Biother Radiopharm. 1999 February; 14(1):31-6.
16. Weigel T L, Lotze M T, Kim P K, Amoscato A A, Luketich J D, Odoux C. Paclitaxel-induced apoptosis in non-small cell lung cancer cell lines is associated with increased caspase-3 activity. J Thorac Cardiovasc Surg. 2000 April; 119(4 Pt 1):795-803.
17. Chang J, Ormerod M, Powles T J, Allred D C, Ashley S E, Dowsett M. Apoptosis and proliferation as predictors of chemotherapy response in patients with breast carcinoma. Cancer. 2000; 89(11):2145-52.
18. C D Archer, M Parton, I E Smith, P A Ellis, J Salter, S Ashley, G Gui, N Sacks, S R Ebbs, W Allum, N Nasiri, and M Dowsett. Early changes in apoptosis and proliferation following primary chemotherapy for breast cancer. Br J Cancer. 2003 Sep. 15; 89(6): 1035-1041.
19. Evans L M, Cowey S L, Siegal G P, Hardy R W. Stearate preferentially induces apoptosis in human breast cancer cells. Nutr Cancer. 2009; 61(5):746-53.
20. Fermor B F, Masters J R, Wood C B, Miller J, Apostolov K, Habib N A. Fatty acid composition of normal and malignant cells and cytotoxicity of stearic, oleic and sterculic acids in vitro. Eur J. Cancer. 1992; 28A:1143-7
21. Dezhi Wang, Cecil R Stockard, Louie Harkins, Patricia Lott, Chura Salih, Kun Yuan, Donald Buchsbaum, Arig Hashim, Majd Zayzafoon, Robert Hardy, Omar Hameed, William Grizzle, and Gene P. Siegal. Immunohistochemistry for the evaluation of angiogenesis in tumor xenografts. Biotech Histochem. 2008 June; 83(3): 179-189.
22. Li C, Zhao X, Toline E, Siegal G P, Evans L M, Ibrahim-Hashim A, Desmond R, Hardy R W. Prevention of carcinogenesis and inhibition of breast cancer tumor burden by dietary stearate. Carcinogenesis. 2011 May 17.
23. Welch D R. Do we need to redefine a cancer metastasis and staging definitions? Breast Dis. 2006; 26:3-12.
24. Steeg P S. Tumor metastasis: mechanistic insights and clinical challenges. Nat Med 2006; 12:895-904.
25. Artwohl M, Roden M, Waldhäusl W, Freudenthaler A, Baumgartner-Parzer S M. Free fatty acids trigger apoptosis and inhibit cell cycle progression in human vascular endothelial cells. FASEB J. 2004 January; 18(1):146-8.
26 Artwohl M, Lindenmair A, Sexl V, Maier C, Rainer G, Freudenthaler A, Huttary N, Wolzt M, Nowotny P, Luger A, Baumgartner-Parzer S M. Different mechanisms of saturated versus polyunsaturated FFA-induced apoptosis in human endothelial cells. J Lipid Res. 2008 December; 49(12):2627-40.
27. Naumova E, Ubezio P, Garofalo A, Borsotti P, Cassis L, Riccardi E, Scanziani E, Eccles S A, Bani M R, Giavazzi R. The vascular targeting property of paclitaxel is enhanced by SU6668, a receptor tyrosine kinase inhibitor, causing apoptosis of endothelial cells and inhibition of angiogenesis. Clin Cancer Res. 2006 Mar. 15; 12(6):1839-49.
28. Dong L F, Swettenham E, Eliasson J, Wang X F, Gold M, Medunic Y, Stantic M, Low P, Prochazka L, Witting P K, Turanek J, Akporiaye E T, Ralph S J, Neuzil J. Vitamin E analogues inhibit angiogenesis by selective induction of apoptosis in proliferating endothelial cells: the role of oxidative stress. Cancer Res. 2007 Dec. 15; 67(24):11906-13.
29. Munaron L. Intracellular calcium, endothelial cells and angiogenesis. Recent Pat Anticancer Drug Discov. 2006 January; 1(1):105-19.
30. Costa I, Moral R, Solanas M, Andreu F J, Ruiz de Villa M C, Escrich E. High corn oil and extra virgin olive oil diets and experimental mammary carcinogenesis: clinicopathological and immunohistochemical p21Ha-Ras expression study. Virchows Arch. 2011 February; 458(2):141-51.
31. Wu B, Iwakiri R, Ootani A, Tsunada S, Fujise T, Sakata Y, Sakata H, Toda S, Fujimoto K. Dietary corn oil promotes colon cancer by inhibiting mitochondria-dependent apoptosis in azoxymethane-treated rats. Exp Biol Med (Maywood). 2004 November; 229(10):1017-25.
32. Wang Z, Pei H, Kaeck M, Lu J. Mammary cancer promotion and MAPK activation associated with consumption of a corn oil-based high-fat diet. Nutr Cancer. 1999; 34(2):140-6.
33. Cheng J L, Futakuchi M, Ogawa K, Iwata T, Kasai M, Tokudome S, Hirose M, Shirai T. Dose response study of conjugated fatty acid derived from safflower oil on mammary and colon carcinogenesis pretreated with 7,12-dimethylbenz[a]anthracene (DMBA) and 1,2-dimethylhydrazine (DMH) in female Sprague-Dawley rats. Cancer Lett. 2003 Jul. 10; 196(2):161-8.
34. Kimoto N, Hirose M, Futakuchi M, Iwata T, Kasai M, Shirai T. Site-dependent modulating effects of conjugated fatty acids from safflower oil in a rat two-stage carcinogenesis model in female Sprague-Dawley rats. Cancer Lett. 2001 Jul. 10; 168(1):15-21.
35. Okuno M, Tanaka T, Komaki C, Nagase S, Shiratori Y, Muto Y, Kajiwara K, Maki T, Moriwaki H. Suppressive effect of low amounts of safflower and perilla oils on diethylnitrosamine-induced hepatocarcinogenesis in male F344 rats. Nutr Cancer. 1998; 30(3):186-93.
36. J Edward Hunter, Jun Zhang, and Penny M Kris-Etherton. Cardiovascular disease risk of dietary stearic acid compared with trans, other saturated, and unsaturated fatty acids: a systematic review. Am J Clin Nutr. 2010; 91:46-63.
37. Weidner N. Current pathologic methods for measuring intratumoral microvessel density within breast carcinoma and other solid tumors. Breast Cancer Res Treat. 1995; 36(2):169-80.

### Example 4

### The Effects of Stearate on Apoptosis Factors cIAP2, BAX, and Bcl-2

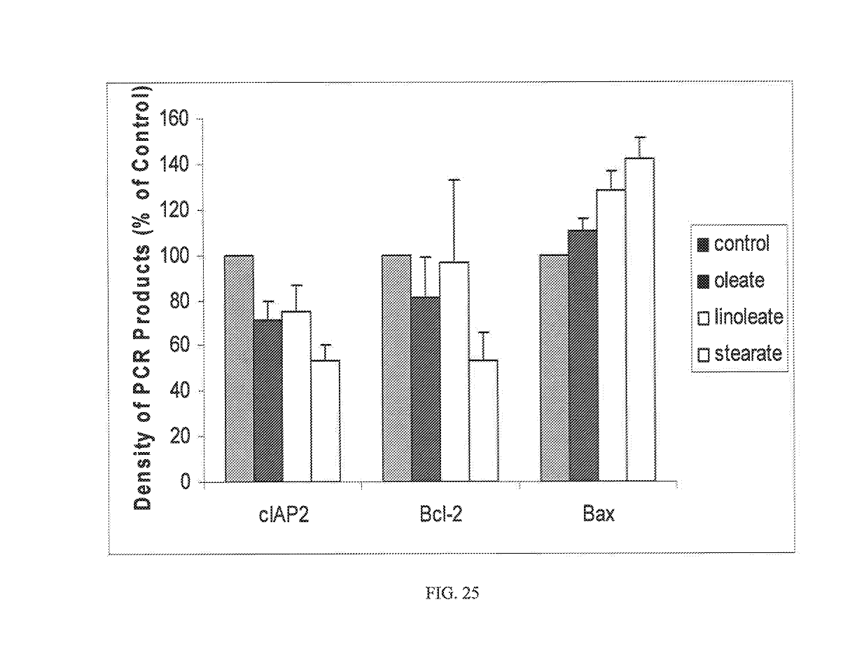

It was hypothesized that stearate induces apoptosis in visceral adipocytes by at least one of the following mechanisms: inhibition of cIAP2, activation of Bcl-2, and activation of BAX. To test these hypotheses, cell cultures of visceral adipocytcs from ApoE knockout mice were exposed to 50 μM oleic acid, 50 04 linoleic acid, 50 μM stearic acid, or no additional fatty acid (control). Six cultures were exposed to each fatty acid or no fatty acid. The expression of cIAP2, Bcl-2, and Bax was measured in each culture. The results, shown in FIG. 25, demonstrate that stearic acid reduces the expression of cIAP2 in visceral adipocyte cells and increases the expression of Bax. Linoleic acid also increased the expression of Bax to a lesser extent but showed no statistically significant effect on cIAP2 expression. cIAP2 and Bcl-2 are inhibitors of apoptosis, while Bax is a pro-apoptotic factor. By decreasing levels of the cIAP2 and/or Bcl-2 polypeptides and increasing expression of the Bax polypeptide, stearate administration may lead to an increase in cellular apoptotic activity (such as but not limited to the visceral fat tissues). Without wishing to be bound by any given hypothetical model, it is proposed that this reduction in cIAP2 expression and this increase in Bax expression (independently or in combination) is responsible for the relative decrease in visceral fat tissue that is observed when animals are given a diet that is high in stearate.

### E. Conclusions

It is to be understood that any given elements of the disclosed embodiments to of the invention may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein.

1. A pharmaceutical preparation comprising a therapeutically effective amount of a stearate compound, wherein said stearate compound is neither a naturally occurring triglyceride compound nor a naturally occurring phospholipid compound.

2. The pharmaceutical preparation of claim 1 comprising an antineoplastic agent.

3. The pharmaceutical preparation of claim 1 comprising an antineoplastic agent selected from the group consisting of: taxane, a taxane derivative, paclitaxel, and docetaxel.

4. The pharmaceutical preparation of claim 1 comprising a taxane derivative having the following structure: 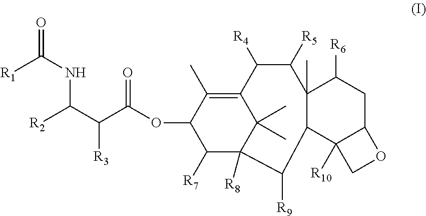
wherein:
R₁, R₂, and R₄-R₈ are unrestricted;
R₃ is hydroxyl or ester;
R₉ is acyl, aroyl, carbonate, or alkyl; and
R₁₀ is acyl, aroyl, carbonate, or alkyl.

5. The pharmaceutical preparation of claim 2 wherein the amount of antineoplastic agent is about 20 mg of antineoplastic agent per kilogram of subject mass.

6. The pharmaceutical preparation of claim 1, wherein the stearate compound is stearic acid or a pharmaceutically acceptable salt thereof.

7. The pharmaceutical preparation of claim 1, wherein the stearate compound is not a stearate ester.

8. The pharmaceutical preparation of claim 1, comprising at least about 2% by weight of the stearate compound or at least about 17% by weight of the stearate compound.

9. (canceled)

10. The pharmaceutical preparation of claim 1, wherein the therapeutically effective amount is an amount effective to reduce the likelihood or severity of tumors in a subject, is an amount effective to reduce the visceral fat content of a subject, is an amount effective to reduce the serum glucose concentration of a subject, reduce the leptin concentration of a subject, or increase serum MCP-1 of a subject, is an amount effective to reduce the serum glucose concentration of a subject, reduce the leptin concentration of a subject, or increase serum MCP-1 of a subject, is an amount effective to reduce the activity in a subject of at least one of RhoA, Rho C, and total Rho, is an amount effective to at least partially arrest at G₁ the cell cycle of a tumor cell in a subject or is an amount effective to increase Ras activity in a subject, increase ERK phosphorylation in a subject, increase p21^{CIP1/WAF1} activity in a subject, increase p27^{KIP}1 activity in a subject, or a combination of the foregoing.

11. (canceled)

12. (canceled)

13. (canceled)

14. (canceled)

15. (canceled)

16. The pharmaceutical preparation of claim 1 for a purpose selected from the group consisting of: reducing visceral fat content, reducing total body fat content, reducing the likelihood or severity of cardiovascular disease, reducing the likelihood or severity of tumorigenesis, reducing serum glucose concentration, reducing leptin concentration, increasing serum MCP-1, and reducing the likelihood or severity of type 2 diabetes.

17. A method of improving or maintaining the health of a subject, the method comprising administering to the subject a stearate compound, other than a naturally occurring triglyceride compound or a naturally occurring phospholipid compound, in an amount equal to a significant fraction of the subject's total dietary lipid intake.

18. The method of claim 17, wherein the stearate compound is administered in the subject's diet.

19. The method of claim 17, wherein the stearate compound is administered in an amount equal to about 27% of the total mass of the subject's total dietary intake or the stearate compound is administered in an amount equal to about 85% of the total mass of the subject's dietary lipid intake.

20. (canceled)

21. The method of claim 17, comprising administering to the subject stearate in the subject's diet equal to about 17% of the total mass of the subject's food intake and administering to the subject an edible oil in an amount equal to about 3% of the total mass of the subject's food intake.

22. The method of claim 17, comprising administering to the subject stearate in the subject's diet equal to about 85% of the mass of the subject's total dietary lipid intake and administering to the subject an edible oil in an amount equal to about 15% of the mass of the subject's total dietary lipid intake.

23. The method of claim 17, wherein the stearate compound is stearic acid or an edible salt thereof.

24. The method of claim 17, wherein the amount is an amount effective to reduce the likelihood or severity of tumors in the subject, is an amount effective to reduce the visceral fat content of a subject, is an amount effective to reduce the serum glucose concentration of a subject, reduce the leptin concentration of a subject, or increase serum MCP-1 of a subject, is an amount effective to reduce the serum glucose concentration of a subject, reduce the leptin concentration of a subject, or increase serum MCP-1 of a subject, is an amount effective to reduce the activity in a subject of at least one of RhoA, Rho C, and total Rho, is an amount effective to at least partially arrest at G₁ the cell cycle of a tumor cell in a subject or is an amount effective to increase Ras activity in a subject, increase ERK phosphorylation in a subject, increase p21^{CIP1/WAF1} activity in a subject, increase p27^{KIP1} activity in a subject, or a combination of the foregoing.

25. (canceled)

26. (canceled)

27. (canceled)

28. (canceled)

29. (canceled)

30. The method of claim 17, wherein the method is for controlling the visceral fat content of the subject, reducing the likelihood or severity of tumorigenesis in the subject, controlling the total body fat content of the subject, reducing the likelihood or severity of cardiovascular disease in the subject, or reducing the likelihood or severity of type-2 diabetes in the subject.

31-41. (canceled)

42. A dietary supplement comprising a substantial amount of a stearate compound, wherein said stearate compound is neither a naturally occurring triglyceride compound nor a naturally occurring phospholipid compound.

43. (canceled)

44. The dietary supplement of claim 42 wherein the stearate compound is stearic acid or a salt thereof.

45. The dietary supplement of claim 42 wherein the stearate compound is not a stearate ester.

46. The dietary supplement of claim 42 wherein the substantial amount is at least about 2% by weight or the substantial amount is at least about 17% by weight.

47. (canceled)

48. (canceled)

49. (canceled)

50. (canceled)

51. (canceled)

52. (canceled)

53. (canceled)

54. (canceled)

55. (canceled)

56. The dietary supplement of claim 42, for a purpose selected from the group consisting of: reducing visceral fat content, reducing total body fat content, reducing the likelihood or severity of cardiovascular disease, reducing the likelihood or severity of tumorigenesis, reducing serum glucose concentration, reducing leptin concentration, increasing serum MCP-1, and reducing the likelihood or severity of type 2 diabetes.
